SEQUENCE LISTING (182 sequences)

SEQ ID NO: 1 - RNA, 14 nt, Artificial
  SYNTHETIC OLIGONUCLEOTIDE
agcuggucaucgcc

SEQ ID NO: 2 - RNA, 37 nt, Artificial
  SYNTHETIC OLIGONUCLEOTIDE
ggcgaucugaugagccgcuucggcggcgaaaccagcu

SEQ ID NO: 3 - RNA, 14 nt, Artificial
  SYNTHETIC OLIGONUCLEOTIDE
acgcagccucuucg

SEQ ID NO: 4 - RNA, 14 nt, Artificial
  SYNTHETIC OLIGONUCLEOTIDE
acaugguucugcug

SEQ ID NO: 5 - RNA, 14 nt, Artificial
  SYNTHETIC OLIGONUCLEOTIDE
ugcuggccuucccc

SEQ ID NO: 6 - RNA, 14 nt, Artificial
  SYNTHETIC OLIGONUCLEOTIDE
ugcuggucuucccc

SEQ ID NO: 7 - RNA, 14 nt, Artificial
  SYNTHETIC OLIGONUCLEOTIDE
gcugggcuuccggc

SEQ ID NO: 8 - RNA, 14 nt, Artificial
  SYNTHETIC OLIGONUCLEOTIDE
ucaccgucuagcac

SEQ ID NO: 9 - RNA, 14 nt, Artificial
  SYNTHETIC OLIGONUCLEOTIDE
agguggcuucacca

SEQ ID NO: 10 - RNA, 14 nt, Artificial
  SYNTHETIC OLIGONUCLEOTIDE
uucuggccccacag

SEQ ID NO: 11 - RNA, 14 nt, Artificial
  SYNTHETIC OLIGONUCLEOTIDE
uggaggacuucuuu

SEQ ID NO: 12 - RNA, 14 nt, Artificial
  SYNTHETIC OLIGONUCLEOTIDE
aucgaguuguacgu

SEQ ID NO: 13 - RNA, 14 nt, Artificial
  SYNTHETIC OLIGONUCLEOTIDE
aucgagccguacgu

SEQ ID NO: 14 - RNA, 14 nt, Artificial
  SYNTHETIC OLIGONUCLEOTIDE
acaucgccgagggc

SEQ ID NO: 15 - RNA, 14 nt, Artificial
  SYNTHETIC OLIGONUCLEOTIDE
aaucggcuacuaca

SEQ ID NO: 16 - RNA, 14 nt, Artificial
  SYNTHETIC OLIGONUCLEOTIDE
ucgugguccgcuuc

SEQ ID NO: 17 - RNA, 14 nt, Artificial
  SYNTHETIC OLIGONUCLEOTIDE
ucgugguccccuuc

SEQ ID NO: 18 - RNA, 14 nt, Artificial
  SYNTHETIC OLIGONUCLEOTIDE
caucuguuucugcu

SEQ ID NO: 19 - RNA, 14 nt, Artificial
  SYNTHETIC OLIGONUCLEOTIDE
agguggccucggcc

SEQ ID NO: 20 - RNA, 13 nt, Artificial
  SYNTHETIC OLIGONUCLEOTIDE
gccacuucgagua

SEQ ID NO: 21 - RNA, 13 nt, Artificial
  SYNTHETIC OLIGONUCLEOTIDE
gccucuucgagua

SEQ ID NO: 22 - RNA, 13 nt, Artificial
  SYNTHETIC OLIGONUCLEOTIDE
cacacuacuaccu

SEQ ID NO: 23 - RNA, 13 nt, Artificial
  SYNTHETIC OLIGONUCLEOTIDE
augguucugcuga

SEQ ID NO: 24 - RNA, 13 nt, Artificial
  SYNTHETIC OLIGONUCLEOTIDE
auguuucggcuga

SEQ ID NO: 25 - RNA, 13 nt, Artificial
  SYNTHETIC OLIGONUCLEOTIDE
ugcgcuuccccau

SEQ ID NO: 26 - RNA, 13 nt, Artificial
  SYNTHETIC OLIGONUCLEOTIDE
uggccuuccccau

SEQ ID NO: 27 - RNA, 13 nt, Artificial
  SYNTHETIC OLIGONUCLEOTIDE
uggucuuccccau

SEQ ID NO: 28 - RNA, 13 nt, Artificial
  SYNTHETIC OLIGONUCLEOTIDE
ugggcuuccgcau

SEQ ID NO: 29 - RNA, 13 nt, Artificial
  SYNTHETIC OLIGONUCLEOTIDE
cuuccucaggcuc

SEQ ID NO: 30 - RNA, 13 nt, Artificial
  SYNTHETIC OLIGONUCLEOTIDE
caggcucuacguc

SEQ ID NO: 31 - RNA, 13 nt, Artificial
  SYNTHETIC OLIGONUCLEOTIDE
caccgucuagcac

SEQ ID NO: 32 - RNA, 13 nt, Artificial
  SYNTHETIC OLIGONUCLEOTIDE
ccgucuagcacaa

SEQ ID NO: 33 - RNA, 13 nt, Artificial
  SYNTHETIC OLIGONUCLEOTIDE
ugaacuacauccu

SEQ ID NO: 34 - RNA, 13 nt, Artificial
  SYNTHETIC OLIGONUCLEOTIDE
ggaccuagguggc

SEQ ID NO: 35 - RNA, 13 nt, Artificial
  SYNTHETIC OLIGONUCLEOTIDE
gugacuucaccag

SEQ ID NO: 36 - RNA, 13 nt, Artificial
  SYNTHETIC OLIGONUCLEOTIDE
cgucuucuggccc

SEQ ID NO: 37 - RNA, 13 nt, Artificial
  SYNTHETIC OLIGONUCLEOTIDE
caggauacaauuu

SEQ ID NO: 38 - RNA, 13 nt, Artificial
  SYNTHETIC OLIGONUCLEOTIDE
aggacuucuuugc

SEQ ID NO: 39 - RNA, 13 nt, Artificial
  SYNTHETIC OLIGONUCLEOTIDE
agggguacguggu

SEQ ID NO: 40 - RNA, 13 nt, Artificial
  SYNTHETIC OLIGONUCLEOTIDE
agugguacguggu

SEQ ID NO: 41 - RNA, 13 nt, Artificial
  SYNTHETIC OLIGONUCLEOTIDE
aguuguacguggu

SEQ ID NO: 42 - RNA, 13 nt, Artificial
  SYNTHETIC OLIGONUCLEOTIDE
agccguacguggu

SEQ ID NO: 43 - RNA, 13 nt, Artificial
  SYNTHETIC OLIGONUCLEOTIDE
uggugucuaagcc

SEQ ID NO: 44 - RNA, 13 nt, Artificial
  SYNTHETIC OLIGONUCLEOTIDE
accccuacucgcc

SEQ ID NO: 45 - RNA, 13 nt, Artificial
  SYNTHETIC OLIGONUCLEOTIDE
ccuacucgccggc

SEQ ID NO: 46 - RNA, 13 nt, Artificial
  SYNTHETIC OLIGONUCLEOTIDE
cacccucacucgc

SEQ ID NO: 47 - RNA, 13 nt, Artificial
  SYNTHETIC OLIGONUCLEOTIDE
gugcauccccgag

SEQ ID NO: 48 - RNA, 13 nt, Artificial
  SYNTHETIC OLIGONUCLEOTIDE
guacaucgccgag

SEQ ID NO: 49 - RNA, 13 nt, Artificial
  SYNTHETIC OLIGONUCLEOTIDE
gcucguauggaau

SEQ ID NO: 50 - RNA, 13 nt, Artificial
  SYNTHETIC OLIGONUCLEOTIDE
ucguguagaaucg

SEQ ID NO: 51 - RNA, 13 nt, Artificial
  SYNTHETIC OLIGONUCLEOTIDE
uggaaucggcuac

SEQ ID NO: 52 - RNA, 13 nt, Artificial
  SYNTHETIC OLIGONUCLEOTIDE
gaaucuacuacua

SEQ ID NO: 53 - RNA, 13 nt, Artificial
  SYNTHETIC OLIGONUCLEOTIDE
cagguucgugguc

SEQ ID NO: 54 - RNA, 13 nt, Artificial
  SYNTHETIC OLIGONUCLEOTIDE
cgugguccgcuuc

SEQ ID NO: 55 - RNA, 13 nt, Artificial
  SYNTHETIC OLIGONUCLEOTIDE
cgugguccccuuc

SEQ ID NO: 56 - RNA, 14 nt, Artificial
  SYNTHETIC OLIGONUCLEOTIDE
ccugaaucugggug

SEQ ID NO: 57 - RNA, 13 nt, Artificial
  SYNTHETIC OLIGONUCLEOTIDE
ggugcucuacgcc

SEQ ID NO: 58 - RNA, 13 nt, Artificial
  SYNTHETIC OLIGONUCLEOTIDE
uaucaucuguuuc

SEQ ID NO: 59 - RNA, 13 nt, Artificial
  SYNTHETIC OLIGONUCLEOTIDE
cuguuucugcuau

SEQ ID NO: 60 - RNA, 13 nt, Artificial
  SYNTHETIC OLIGONUCLEOTIDE
ucuuuuccgcuau

SEQ ID NO: 61 - RNA, 13 nt, Artificial
  SYNTHETIC OLIGONUCLEOTIDE
agaguucuuugcc

SEQ ID NO: 62 - RNA, 13 nt, Artificial
  SYNTHETIC OLIGONUCLEOTIDE
cgagcuagguggc

SEQ ID NO: 63 - RNA, 13 nt, Artificial
  SYNTHETIC OLIGONUCLEOTIDE
uggccucggccua

SEQ ID NO: 64 - RNA, 14 nt, Artificial
  SYNTHETIC OLIGONUCLEOTIDE
aaaaaaucuugaca

SEQ ID NO: 65 - RNA, 14 nt, Artificial
  SYNTHETIC OLIGONUCLEOTIDE
ggcucucugcuuuc

SEQ ID NO: 66 - RNA, 13 nt, Artificial
  SYNTHETIC OLIGONUCLEOTIDE
gguuuucaggacu

SEQ ID NO: 67 - RNA, 13 nt, Artificial
  SYNTHETIC OLIGONUCLEOTIDE
gguuuuugggacu

SEQ ID NO: 68 - RNA, 13 nt, Artificial
  SYNTHETIC OLIGONUCLEOTIDE
guccguuucagcu

SEQ ID NO: 69 - RNA, 13 nt, Artificial
  SYNTHETIC OLIGONUCLEOTIDE
gcugcuccaaucc

SEQ ID NO: 70 - RNA, 13 nt, Artificial
  SYNTHETIC OLIGONUCLEOTIDE
aaucuuagcucgc

SEQ ID NO: 71 - RNA, 13 nt, Artificial
  SYNTHETIC OLIGONUCLEOTIDE
cuagcucgcggcc

SEQ ID NO: 72 - RNA, 14 nt, Artificial
  SYNTHETIC OLIGONUCLEOTIDE
ucucugcuuucugc

SEQ ID NO: 73 - RNA, 14 nt, Artificial
  SYNTHETIC OLIGONUCLEOTIDE
caacgguuuuuggg

SEQ ID NO: 74 - RNA, 14 nt, Artificial
  SYNTHETIC OLIGONUCLEOTIDE
cguccguuucagcu

SEQ ID NO: 75 - RNA, 14 nt, Artificial
  SYNTHETIC OLIGONUCLEOTIDE
cagcugcuccaauc

SEQ ID NO: 76 - RNA, 14 nt, Artificial
  SYNTHETIC OLIGONUCLEOTIDE
ucuuagcucgccac

SEQ ID NO: 77 - RNA, 14 nt, Artificial
  SYNTHETIC OLIGONUCLEOTIDE
uccuagcucgcggc

SEQ ID NO: 78 - RNA, 13 nt, Artificial
  SYNTHETIC OLIGONUCLEOTIDE
aggcgucagccua

SEQ ID NO: 79 - RNA, 13 nt, Artificial
  SYNTHETIC OLIGONUCLEOTIDE
uggccucggccua

SEQ ID NO: 80 - RNA, 12 nt, Artificial
  SYNTHETIC OLIGONUCLEOTIDE
caugucucuuug

SEQ ID NO: 81 - RNA, 14 nt, Artificial
  SYNTHETIC OLIGONUCLEOTIDE
ggccuguccuugga

SEQ ID NO: 82 - RNA, 13 nt, Artificial
  SYNTHETIC OLIGONUCLEOTIDE
agaugucuauaag

SEQ ID NO: 83 - RNA, 13 nt, Artificial
  SYNTHETIC OLIGONUCLEOTIDE
gagagucucauga

SEQ ID NO: 84 - RNA, 13 nt, Artificial
  SYNTHETIC OLIGONUCLEOTIDE
gcgcgucuucccg

SEQ ID NO: 85 - RNA, 13 nt, Artificial
  SYNTHETIC OLIGONUCLEOTIDE
acuggucuucuac

SEQ ID NO: 86 - RNA, 13 nt, Artificial
  SYNTHETIC OLIGONUCLEOTIDE
cuaugccuucaug

SEQ ID NO: 87 - RNA, 13 nt, Artificial
  SYNTHETIC OLIGONUCLEOTIDE
cgcugucuuccac

SEQ ID NO: 88 - RNA, 13 nt, Artificial
  SYNTHETIC OLIGONUCLEOTIDE
cuucgucuuugca

SEQ ID NO: 89 - RNA, 13 nt, Artificial
  SYNTHETIC OLIGONUCLEOTIDE
guacgucuuccau

SEQ ID NO: 90 - RNA, 34 nt, Artificial
  SYNTHETIC OLIGONUCLEOTIDE
caaagacugaugagccguucgcggcgaaacaugu

SEQ ID NO: 91 - RNA, 34 nt, Artificial
  SYNTHETIC OLIGONUCLEOTIDE
ggcauacugaugagccguucgcggcgaaacaaug

SEQ ID NO: 92 - RNA, 34 nt, Artificial
  SYNTHETIC OLIGONUCLEOTIDE
caugaacugaugagccguucgcggcgaaacauag

SEQ ID NO: 93 - RNA, 34 nt, Artificial
  SYNTHETIC OLIGONUCLEOTIDE
guggaacugaugagccguucgcggcgaaacagcg

SEQ ID NO: 94 - RNA, 34 nt, Artificial
  SYNTHETIC OLIGONUCLEOTIDE
gagguacugacgagccguucgcggcgaaacagca

SEQ ID NO: 95 - RNA, 34 nt, Artificial
  SYNTHETIC OLIGONUCLEOTIDE
guggcacugaugagccguucgcggcgaaacagga

SEQ ID NO: 96 - RNA, 34 nt, Artificial
  SYNTHETIC OLIGONUCLEOTIDE
cuuauacugaugagccguucgcggcgaaacaucu

SEQ ID NO: 97 - RNA, 34 nt, Artificial
  SYNTHETIC OLIGONUCLEOTIDE
ucaugacugaugagccguucgcggcgaaacucuc

SEQ ID NO: 98 - RNA, 34 nt, Artificial
  SYNTHETIC OLIGONUCLEOTIDE
cgggaacugaugagccguucgcggcgaaacgcgc

SEQ ID NO: 99 - RNA, 34 nt, Artificial
  SYNTHETIC OLIGONUCLEOTIDE
guagaacugaugagccguucgcggcgaaaccagu

SEQ ID NO: 100 - RNA, 34 nt, Artificial
  SYNTHETIC OLIGONUCLEOTIDE
ugcaaacugaugagccguucgcggcgaaacgaag

SEQ ID NO: 101 - RNA, 32 nt, Artificial
  SYNTHETIC OLIGONUCLEOTIDE
ggaacugaugagccguucgcggcgaaacguac

SEQ ID NO: 102 - RNA, 35 nt, Artificial
  SYNTHETIC OLIGONUCLEOTIDE
uaggcucugaugagccgcuucggcggcaaacgccu

SEQ ID NO: 103 - RNA, 35 nt, Artificial
  SYNTHETIC OLIGONUCLEOTIDE
uaggcccugaugagccgcuucggcggcaaaggcca

SEQ ID NO: 104 - RNA, 37 nt, Artificial
  SYNTHETIC OLIGONUCLEOTIDE
gccccaagcugaugagccgcuucggcggcgaaacagg

SEQ ID NO: 105 - RNA, 14 nt, Artificial
  SYNTHETIC OLIGONUCLEOTIDE
nnnynghybnnnnn

SEQ ID NO: 106 - RNA, 57 nt, Artificial
  SYNTHETIC OLIGONUCLEOTIDE
nnnnnvagaagnnnaccagagaaacacagcacgaaagugcugguacauuaccuggua

SEQ ID NO: 107 - RNA, 37 nt, Artificial
  SYNTHETIC OLIGONUCLEOTIDE
nnnnnnncugaugagccgcuucggcggcgaannnnnn

SEQ ID NO: 108 - RNA, 14 nt, Artificial
  SYNTHETIC OLIGONUCLEOTIDE
cgaagaagaagcgu

SEQ ID NO: 109 - RNA, 14 nt, Artificial
  SYNTHETIC OLIGONUCLEOTIDE
cagcagagaagugu

SEQ ID NO: 110 - RNA, 14 nt, Artificial
  SYNTHETIC OLIGONUCLEOTIDE
ggggaaagaaggca

SEQ ID NO: 111 - RNA, 14 nt, Artificial
  SYNTHETIC OLIGONUCLEOTIDE
ggggaaagaaggca

SEQ ID NO: 112 - RNA, 14 nt, Artificial
  SYNTHETIC OLIGONUCLEOTIDE
gccggaagaacagc

SEQ ID NO: 113 - RNA, 14 nt, Artificial
  SYNTHETIC OLIGONUCLEOTIDE
gugcuaagaaguga

SEQ ID NO: 114 - RNA, 14 nt, Artificial
  SYNTHETIC OLIGONUCLEOTIDE
uggugaagaagccu

SEQ ID NO: 115 - RNA, 14 nt, Artificial
  SYNTHETIC OLIGONUCLEOTIDE
cuguggagaaggaa

SEQ ID NO: 116 - RNA, 14 nt, Artificial
  SYNTHETIC OLIGONUCLEOTIDE
aaagaaagaaucca

SEQ ID NO: 117 - RNA, 14 nt, Artificial
  SYNTHETIC OLIGONUCLEOTIDE
acguacagaacgau

SEQ ID NO: 118 - RNA, 14 nt, Artificial
  SYNTHETIC OLIGONUCLEOTIDE
acguacagaacgau

SEQ ID NO: 119 - RNA, 14 nt, Artificial
  SYNTHETIC OLIGONUCLEOTIDE
gcccucagaagugu

SEQ ID NO: 120 - RNA, 14 nt, Artificial
  SYNTHETIC OLIGONUCLEOTIDE
uguaguagaagauu

SEQ ID NO: 121 - RNA, 14 nt, Artificial
  SYNTHETIC OLIGONUCLEOTIDE
gaagcgagaagcga

SEQ ID NO: 122 - RNA, 14 nt, Artificial
  SYNTHETIC OLIGONUCLEOTIDE
gaagggagaagcga

SEQ ID NO: 123 - RNA, 14 nt, Artificial
  SYNTHETIC OLIGONUCLEOTIDE
agcagaagaagaug

SEQ ID NO: 124 - RNA, 14 nt, Artificial
  SYNTHETIC OLIGONUCLEOTIDE
ggccgaagaagccu

SEQ ID NO: 125 - RNA, 12 nt, Artificial
  SYNTHETIC OLIGONUCLEOTIDE
uacucgaguggc

SEQ ID NO: 126 - RNA, 12 nt, Artificial
  SYNTHETIC OLIGONUCLEOTIDE
uacucgagaggc

SEQ ID NO: 127 - RNA, 12 nt, Artificial
  SYNTHETIC OLIGONUCLEOTIDE
agggagagugug

SEQ ID NO: 128 - RNA, 12 nt, Artificial
  SYNTHETIC OLIGONUCLEOTIDE
ucagcaaaccau

SEQ ID NO: 129 - RNA, 12 nt, Artificial
  SYNTHETIC OLIGONUCLEOTIDE
ucagccaaacau

SEQ ID NO: 130 - RNA, 12 nt, Artificial
  SYNTHETIC OLIGONUCLEOTIDE
auggggagcgca

SEQ ID NO: 131 - RNA, 12 nt, Artificial
  SYNTHETIC OLIGONUCLEOTIDE
auggggaggcca

SEQ ID NO: 132 - RNA, 12 nt, Artificial
  SYNTHETIC OLIGONUCLEOTIDE
auggggagacca

SEQ ID NO: 133 - RNA, 12 nt, Artificial
  SYNTHETIC OLIGONUCLEOTIDE
augcggagccca

SEQ ID NO: 134 - RNA, 12 nt, Artificial
  SYNTHETIC OLIGONUCLEOTIDE
gagccuaggaag

SEQ ID NO: 135 - RNA, 12 nt, Artificial
  SYNTHETIC OLIGONUCLEOTIDE
gacguaagccug

SEQ ID NO: 136 - RNA, 12 nt, Artificial
  SYNTHETIC OLIGONUCLEOTIDE
gugcuaacggug

SEQ ID NO: 137 - RNA, 12 nt, Artificial
  SYNTHETIC OLIGONUCLEOTIDE
gugcuaacggug

SEQ ID NO: 138 - RNA, 12 nt, Artificial
  SYNTHETIC OLIGONUCLEOTIDE
aggaugaguuca

SEQ ID NO: 139 - RNA, 12 nt, Artificial
  SYNTHETIC OLIGONUCLEOTIDE
gccaccaggucc

SEQ ID NO: 140 - RNA, 12 nt, Artificial
  SYNTHETIC OLIGONUCLEOTIDE
cuggugagucac

SEQ ID NO: 141 - RNA, 12 nt, Artificial
  SYNTHETIC OLIGONUCLEOTIDE
gggccaaagacg

SEQ ID NO: 142 - RNA, 12 nt, Artificial
  SYNTHETIC OLIGONUCLEOTIDE
aaauugauccug

SEQ ID NO: 143 - RNA, 12 nt, Artificial
  SYNTHETIC OLIGONUCLEOTIDE
gcaaagaguccu

SEQ ID NO: 144 - RNA, 12 nt, Artificial
  SYNTHETIC OLIGONUCLEOTIDE
accacgaccccu

SEQ ID NO: 145 - RNA, 12 nt, Artificial
  SYNTHETIC OLIGONUCLEOTIDE
accacgaccacu

SEQ ID NO: 146 - RNA, 12 nt, Artificial
  SYNTHETIC OLIGONUCLEOTIDE
accacgacaacu

SEQ ID NO: 147 - RNA, 12 nt, Artificial
  SYNTHETIC OLIGONUCLEOTIDE
accacgacggcu

SEQ ID NO: 148 - RNA, 12 nt, Artificial
  SYNTHETIC OLIGONUCLEOTIDE
ggcuuaacacca

SEQ ID NO: 149 - RNA, 12 nt, Artificial
  SYNTHETIC OLIGONUCLEOTIDE
ggcgagaggggu

SEQ ID NO: 150 - RNA, 12 nt, Artificial
  SYNTHETIC OLIGONUCLEOTIDE
gccggcaguagg

SEQ ID NO: 151 - RNA, 12 nt, Artificial
  SYNTHETIC OLIGONUCLEOTIDE
gcgaguagggug

SEQ ID NO: 152 - RNA, 12 nt, Artificial
  SYNTHETIC OLIGONUCLEOTIDE
cucgggaugcac

SEQ ID NO: 153 - RNA, 12 nt, Artificial
  SYNTHETIC OLIGONUCLEOTIDE
cucggcauguac

SEQ ID NO: 154 - RNA, 12 nt, Artificial
  SYNTHETIC OLIGONUCLEOTIDE
auuccaacgagc

SEQ ID NO: 155 - RNA, 12 nt, Artificial
  SYNTHETIC OLIGONUCLEOTIDE
cgauucacacga

SEQ ID NO: 156 - RNA, 12 nt, Artificial
  SYNTHETIC OLIGONUCLEOTIDE
guagccauucca

SEQ ID NO: 157 - RNA, 12 nt, Artificial
  SYNTHETIC OLIGONUCLEOTIDE
uaguagagauuc

SEQ ID NO: 158 - RNA, 12 nt, Artificial
  SYNTHETIC OLIGONUCLEOTIDE
gaccacaaccug

SEQ ID NO: 159 - RNA, 12 nt, Artificial
  SYNTHETIC OLIGONUCLEOTIDE
gaagcgaccacg

SEQ ID NO: 160 - RNA, 12 nt, Artificial
  SYNTHETIC OLIGONUCLEOTIDE
gaagggaccacg

SEQ ID NO: 161 - RNA, 13 nt, Artificial
  SYNTHETIC OLIGONUCLEOTIDE
cacccaauucagg

SEQ ID NO: 162 - RNA, 12 nt, Artificial
  SYNTHETIC OLIGONUCLEOTIDE
ggcguaagcacc

SEQ ID NO: 163 - RNA, 12 nt, Artificial
  SYNTHETIC OLIGONUCLEOTIDE
gaaacaaugaua

SEQ ID NO: 164 - RNA, 12 nt, Artificial
  SYNTHETIC OLIGONUCLEOTIDE
auagcaaaacag

SEQ ID NO: 165 - RNA, 12 nt, Artificial
  SYNTHETIC OLIGONUCLEOTIDE
auagcgaacaga

SEQ ID NO: 166 - RNA, 12 nt, Artificial
  SYNTHETIC OLIGONUCLEOTIDE
ggcaaaaacucu

SEQ ID NO: 167 - RNA, 12 nt, Artificial
  SYNTHETIC OLIGONUCLEOTIDE
uccaccagcucg

SEQ ID NO: 168 - RNA, 12 nt, Artificial
  SYNTHETIC OLIGONUCLEOTIDE
uaggccaggcca

SEQ ID NO: 169 - RNA, 13 nt, Artificial
  SYNTHETIC OLIGONUCLEOTIDE
ugucaaauuuuuu

SEQ ID NO: 170 - RNA, 13 nt, Artificial
  SYNTHETIC OLIGONUCLEOTIDE
gaaagcaagagcc

SEQ ID NO: 171 - RNA, 12 nt, Artificial
  SYNTHETIC OLIGONUCLEOTIDE
aguccuaaaacc

SEQ ID NO: 172 - RNA, 12 nt, Artificial
  SYNTHETIC OLIGONUCLEOTIDE
agucccaaaacc

SEQ ID NO: 173 - RNA, 12 nt, Artificial
  SYNTHETIC OLIGONUCLEOTIDE
agcugaacggac

SEQ ID NO: 174 - RNA, 12 nt, Artificial
  SYNTHETIC OLIGONUCLEOTIDE
ggauugagcagc

SEQ ID NO: 175 - RNA, 12 nt, Artificial
  SYNTHETIC OLIGONUCLEOTIDE
acgagcaagauu

SEQ ID NO: 176 - RNA, 12 nt, Artificial
  SYNTHETIC OLIGONUCLEOTIDE
ggccgcagcuag

SEQ ID NO: 177 - RNA, 14 nt, Artificial
  SYNTHETIC OLIGONUCLEOTIDE
gcagaaagaagaga

SEQ ID NO: 178 - RNA, 14 nt, Artificial
  SYNTHETIC OLIGONUCLEOTIDE
cccaaaagaaguug

SEQ ID NO: 179 - RNA, 14 nt, Artificial
  SYNTHETIC OLIGONUCLEOTIDE
agcugaagaagacg

SEQ ID NO: 180 - RNA, 14 nt, Artificial
  SYNTHETIC OLIGONUCLEOTIDE
gauuggagaagcug

SEQ ID NO: 181 - RNA, 14 nt, Artificial
  SYNTHETIC OLIGONUCLEOTIDE
guggcgagaagaga

SEQ ID NO: 182 - RNA, 14 nt, Artificial
  SYNTHETIC OLIGONUCLEOTIDE
gccgcgagaaggga

(19) United States
(12) Patent Application Publication
(10) Pub. No.: US 2005/0096282 A1
(43) Pub. Date: May 5, 2005

(54) Adeno-associated virus-delivered ribozyme compositions and methods for the treatment of retinal diseases

(76) Inventors: Alfred S. Lewin, Gainesville, FL (US); Lynn C. Shaw, Gainesville, FL (US); Maria B. Grant, (US)

Correspondence Address: WILLIAMS, MORGAN & AMERSON, P.C., 10333 RICHMOND, SUITE 1100, HOUSTON, TX, 77042, US

(21) Appl. No.: 09/847,601

(22) Filed: May 1, 2001

Related U.S. Application Data
(63) Continuation-in-part of application No. 09/063,667, filed on Apr. 21, 1998, now Pat. No. 6,225,291.
(60) Provisional application No. 60/046,147, filed on May 9, 1997.
(60) Provisional application No. 60/044,492, filed on Apr. 21, 1997.

(51) Int. Cl. A61K 48/00; C07H 21/02

(52) U.S. Cl. 514/44; 536/23.1

(57) ABSTRACT

Disclosed are ribozymes, as well as compositions, vectors, virus particles, host cells, and therapeutic kits comprising them useful in the treatment of diseases of the eye, including retinopathy and macular degeneration, and the amelioration of symptoms of such diseases including loss of vision, retinitis, and blindness.

### 1. BACKGROUND OF THE INVENTION

The present application is a continuation-in-part of co-pending application Ser. No. 09/063,667, filed Apr. 21, 1998, to issue May 1, 2001 as U.S. Pat. No. 6,225,291, which claimed priority from provisional application Ser. No. 60/046,147, filed May 9, 1997, and provisional application Ser. No. 60/044,492, filed Apr. 21, 1997, each now abandoned; the entire contents of each of which is specifically incorporated herein by reference in its entirety. The United States government has certain rights in the present invention pursuant to grant number EY08571 from the National Institutes of Health.

1.1 Field of the Invention

The present invention relates generally to the fields of genetics, molecular and cellular biology and medicine. More particularly, it concerns ribozymes, as well as AAV-based vectors, virus, host cells, and kits comprising them, as well as methods for their use in treating or reducing the severity or symptoms of a variety of diseases of the mammalian eye, including, for example, retinal degeneration, retinitis pigmentosa, macular degeneration, and retinopathy.

1.2, Description of Related Art

1.2.1 Ribozymes

Ribozymes are biological catalysts consisting of only RNA. They promote a variety of reactions involving RNA and DNA molecules including site-specific cleavage, ligation, polymerization, and phosphoryl exchange (Cech, 1989; Cech, 1990). Ribozymes fall into three broad classes: (1) RNAse P, (2) self-splicing introns, and (3) self-cleaving viral agents. Self-cleaving agents include hepatitis delta virus and components of plant virus satellite RNAs that sever the RNA genome as part of a rolling-circle mode of replication. Because of their small size and great specificity, ribozymes have the greatest potential for biotechnical applications. The ability of ribozymes to cleave other RNA molecules at specific sites in a catalytic manner has brought them into consideration as inhibitors of viral replication or of cell proliferation and gives them potential advantage over antisense RNA. Indeed, ribozymes have already been used to cleave viral targets and oncogene products in living cells (Koizumi et al., 1992; Kashani-Sabet et al., 1992; Taylor and Rossi, 1991; von-Weizsacker et al., 1992; Ojwang et al., 1992; Stephenson and Gibson, 1991; Yu et al., 1993; Xing and Whitton, 1993; Yu et al., 1995; Little and Lee, 1995).

Two kinds of ribozymes have been employed widely, hairpins and hammerheads. Both catalyze sequence-specific cleavage resulting in products with a 5′ hydroxyl and a 2′,3′-cyclic phosphate. Hammerhead ribozymes have been used more commonly, because they impose few restrictions on the target site. Hairpin ribozymes are more stable and, consequently, function better than hammerheads at physiologic temperature and magnesium concentrations.

A number of patents have issued describing various ribozymes and methods for designing ribozymes. See, for example, U.S. Pat. Nos. 5,646,031; 5,646,020; 5,639,655; 5,093,246; 4,987,071; 5,116,742; and 5,037,746, each specifically incorporated herein by reference in its entirety. However, the ability of ribozymes to provide therapeutic benefit in vivo has not yet been demonstrated.

1.2.2 Diseases of the Eye

There are more than 200 inherited diseases that lead to retinal degeneration in humans. Considerable progress has been made in identifying genes and mutations causing many forms of inherited retinal degeneration in humans and other animals. Diseases causing inherited retinal degeneration in humans can be classified broadly into those that first affect peripheral vision and the peripheral retina, such as retinitis pigmentosa, and those that primarily affect central vision and the macula, such as macular dystrophy. The macula has the highest concentration of cones and the peripheral retina is dominated by rods.

Retinitis pigmentosa (RP) is a collection of heritable retinal degenerations caused by defects in one of several genes for proteins of photoreceptor (PR) cells. RP is characterized by progressive rod photoreceptor degeneration and eventual blindness. The exact molecular pathogenesis of RP is still unexplained. Ultrastructural observations suggest that the rod PRs are severely affected in the disease. Approximately 50,000 individuals in the United States are estimated to have RP. The clinical symptoms of retinitis pigmentosa include night blindness and loss of peripheral vision. With time visual impairment progresses toward the center of the retina causing “tunnel-vision.”

Retinitis pigmentosa can be subdivided into several genetic categories: antosomal dominant (adRP), autosomal recessive (arRP), X-linked (xIRP) or syndromic. There are also a number of clinical classes for retinitis pigmentosa. These classes have been condensed into two broad categories. Type 1 retinitis pigmentosa is characterized by rapid progression and diffuse, severe pigmentation; type 2 retinitis pigmentosa has a slower progression and more regional, less severe pigmentation.

Macular degeneration is a deterioration of the macula (the cone-rich center of vision) leading to gradual loss of central vision. Eventual loss of these cones leads to central vision loss and functional blindness. At least 500,000 individuals are estimated to suffer from macular degeneration currently in the United States. Macular degeneration can have either a genetic basis or it may be an acquired disease. Approximately 10% of Americans over the age of 50 are afflicted with age-related macular degeneration, an acquired form of disease. The inherited forms of macular degeneration are much less common but usually more severe. Inherited macular degeneration is characterized by early development of macular abnormalities such as yellowish deposits and atrophic or pigmented lesions, followed by progressive loss of central vision.

There are many other inherited diseases that also cause retinal degeneration in humans. Among these are gyrate atrophy, Norrie disease, choroideremia and various cone-rod dystrophies. In addition there are numerous inherited systemic diseases, such as Bardet-Biedl, Charcot-Marie-Tooth, and Refsum disease which include retinal degeneration among a multiplicity of other symptoms.

Another important ocular disease is diabetic retinopathy, the leading cause of blindness in adults between the ages of 18 and 72. Histological studies consistently implicate endothelial cell dysfunction in the pathology. A hallmark of advancing diabetic retinopathy is aberrant retinal neovascularization, termed proliferative diabetic retinopathy (PDR).

Hyperglycemia directly contributes to the development of diabetic retinopathy, and early in the development of diabetic retinopathy there exists disruption of the blood-retinal barrier. NOS activity, as determined by conversion of arginine to citrulline, is significantly increased in diabetes Rosen et al., 1995). Gade and coworkers demonstrated that endothelial cell dysfunction correlated with elevated glucose in an in vitro wound model and was mediated by increased levels of NO (Gade et al., 1997). In rat cerebral arteries acute glucose exposure dilates arteries via an endothelium mediated mechanism that involves NO (Cipolla et al., 1997). Cosentino demonstrated that prolonged exposure to high glucose increases eNOS gene expression, protein synthesis, and NO release (Cosentino et al., 1997).

Nitric oxide (NO) is a pleiotropic molecule with multiple physiological effects: neurotransmitter, component of the immune defense system, regulator of smooth muscle tone and blood pressure, inhibitor of platelet aggregation and a superoxide scavenger. NO is synthesized as a product of the conversion of L-arginine into L-citrulline by the so-called constitutive nitric oxide synthase (NOS), either neuronal (nNOS) or endothelial (eNOS) isoforms. NO regulates specific protein levels. NO increases mRNA levels for VEGF and iNOS.

Although several studies on NO function in the retina have been published, very little information is available pertaining to its role in the diabetic retina (Chakravarthy et al., 1995; Goldstein et al., 1996). The iNOS isoform is expressed in the retina, as shown by RT-PCR™ and immunocytochemistry. It is believed to be involved in the development of diabetic retinopathy and in ischemia-reperfusion injury (Hangai et al., 1996; Ostwald et al., 1995). Administering NOS inhibitors can ameliorate or prevent ischemia-reperfusion injury (Lam and Tso, 1996). Diabetic human retinal pigmented epithelial cells have augmented iNOS compared to non-diabetic cells. An increasing body of evidence indicates growth factors including vascular endothelial growth factor (VEGF) and insulin-like growth factor-I (IGF-I) are involved in increased permeability of endothelium that leads to breakdown of the blood-retinal barrier in this microvascular disease. However, the mechanisms for growth factor action in disease progression remain elusive.

1.2.3 Deficiencies in the Prior Art

There is currently no effective treatment for most forms of retinitis pigmentosa or macular degeneration. Treatment with a massive supplement (15,000 I.U. per day) of vitamin A often retards the course of retinal degeneration in retinitis pigmentosa. Vitamin therapy does not treat the underlying cause of RP, and is not a cure.

Also what are lacking are feasible approaches for the systemic or local administration of retinal therapeutic agents that can halt or prevent damage from retinal diseases, including for example, neovascularization in patients with diabetic retinopathy. Although considerable attention has been given to vascular endothelial growth factor (VEGF), an increasing body of evidence implicates insulin-like growth factor-I (IGF-I) in the pathogenesis of aberrant neovascularization that characterizes PDR (Frank, 1990; Smith et al., 1997). It has been demonstrated that the adenosine A_{2B} receptor is expressed in angiogenic blood vessels, and that activation of this receptor results in local VEGF and IGF-I production. Adenosine acting through A_{2B} receptors links altered cellular metabolism caused by oxygen deprivation to compensatory angiogenesis. Adaptation to hypoxia includes induction of diverse genes that appear to depend on a common mode of oxygen sensing and signal transduction, triggering the activation of critical transcription factors, hypoxia-inducible factors (HIFs). The development of targeted gene therapy methods to address such limitations in the art, and to develop therapeutic compositions to effectively treat diseases of the mammalian retina would represent a significant advancement in the fields of medicine and, in particular, ophthalmology and the treatment of disorders and diseases of the human eye.

### 2.0 SUMMARY OF THE INVENTION

The present invention overcomes these and other inherent limitations in the prior art, by providing materials and methods for the treatment of diseases of the mammalian eye. More specifically, the subject invention provides polynucleotide sequences, and methods for using these sequences, to achieve highly specific degradation or reduction of mRNAs encoding polypeptides that cause, contribute to, or participate in disease and dysfunction of the eye, and in particular, the retina. As described herein, the materials and methods of the subject invention can be used to treat a variety of ophthalmic disorers and diseases. In preferred embodiments, the invention provides compositions, methods, and therapeutic kits for treatment and/or the amelioration of symptoms of diseases and disorders of the human eye, such as for example, retinitis, retinitis pigmentosa (RP), autosomal dominant retinitis pigmentosa (ADRP), retinopathy, diabetic retinopathy, macular degeneration, age-related macular degeneration, and a variety of related disorders.

In particular illustrative embodiments described herein, the subject invention employs the use of novel catalytic ribonucleotide compounds, and in particular, hammerhead and/or hairpin ribozymes, that have been designed to cleave mutant forms of messenger RNA (mRNA) occurring in various forms of ocular diseases and retinal damage or degeneration. These ribozyme compounds have been designed and particularly selected such that the catalytic domain of each ribozyme has highly effective, stable, selective activity in cleaving target mRNAs to bring about a reduction in, or an elimination of, the encoded polypeptide produced from translation of the mRNA by cellular protein synthesis machinery.

In a first embodiment, the present invention provides a ribozyme that specifically cleaves an mRNA encoding a polypeptide that causes or contributes to the disease, disorder, or dysfunction of a cell or a tissue of a mammalian eye.

Preferred ribozymes include those catalytic RNA molecules, that specifically cleave an mRNA encoding a polypeptide selected from the group consisting of rod opsin, RP1, RDS/Peripherin, iNOS, A_{2B}, IGF-1, alpha 1, alpha 3, and alpha V. Exemplary ribozymes of the invention include those that comprise the nucleotide sequence of any one of SEQ ID NO:2 and SEQ ID NO:90 to SEQ ID NO:105, and those ribozymes that specifically cleave an mRNA that comprises a sequence selected from any one of SEQ ID NO:3 to SEQ ID NO:89.

Exemplary ribozymes of the invention are shown in SEQ ID NO:2, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, and SEQ ID NO:105.

Preferred ribozymes of the invention encompass those catalytic RNA molecules that specifically cleave an mRNA that encodes a polypeptide selected from the group consisting of a mutant rod opsin polypeptide, a mutant RP1 polypeptide, a mutant RDS/Peripherin polypeptide, a mutant iNOS polypeptide, a mutant A_{2B} polypeptide, a mutant IGF-1 polypeptide, a mutant alpha 1 polypeptide, a mutant alpha 3 polypeptide, and a mutant alpha V polypeptide.

Exemplary ribozymes preferred in the practice of the invention include those that specifically cleave an mRNA encoding a mutant rod opsin polypeptide that comprises a mutation selected from the group consisting of P23H, P23L, Q28H, F45L, L46R, G51A, G51G, G51R, G51V, P53R, T58R, Q64stop, 68-71, V87D, G90D, G106W, C110Y, G114D, R135G, R135L, R135P, P171L, P171S, Y178C, P180A, C187Y, G188R, D190G, D190Y, M207R, H211R, H211P, F220C, C264X, P267L, F220C, C222R, A292E, Q344stop, and P347S. Such designations follow the standard protein nomenclature, in that a “P23H” mutation is one in which the native amino acid at position 23 of the polypeptide (in this case Pro) is changed via mutagenesis to a His. Likewise, an F200C mutant, is a peptide where the Phe at position 200 is changed to a Cys residue at that position, and so forth.

Exemplary ribozymes include those catalytic RNA molecules that specifically cleave mRNAs that comprise a nucleotide sequence selected from the group consisting of SEQ ID NO:1, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, and SEQ ID NO:63.

Likewise, an exemplary ribozyme that specifically cleaves an mRNA encoding a mutant RP1 polypeptide is one that specifically cleaves an mRNA comprising the sequence of SEQ ID NO:64.

Exemplary ribozymes that specifically cleave an mRNA encoding a mutant RDS/Peripherin polypeptide include those ribozymes that specifically cleave an mRNA encoding a mutant RDS/Peripherin polypeptide that comprises a mutation selected from the group consisting of C118, R172Q, R172W, P210R, C214S, P216L, and P219.

Such preferred ribozymes include those that specifically cleave an mRNA that comprises a sequence selected from the group consisting of SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, and SEQ ID NO:77.

As described herein, the ribozymes of the present invention may be of the hairpin (FIG. 11) or the hammerhead (FIG. 12) variety.

A further aspect of the invention is a vector, virus, or host cell that comprises a polynucleotide encoding one or more such preferred ribozymes. Such vectors, virus and host cells will preferably comprise at least a first such polynucleotide that is operably linked to at least a first promoter element that directs expression of the polynucleotide in a mammalian cell to produce the desired ribozyme. Such vectors may include viral vectors such as adenoviral or adeno-associated viral vectors, and such promoter elements will preferably direct the expression of the polynucleotide in a cells and/or tissues of a mammalian, and in particular, a human eye. Exemplary host cells include retinal cells, photoreceptor cells, rod cells, cone cells, Mueller cells, and retinal pigement epithelial cells. Such vectors may include promoter element that comprise a constitutive or an inducible promoter element operable in the eye, such as, for example, a CMV promoter, a mammalian rod opsin promoter, or other suitable promoter element.

The invention also encompasses compositions, formulations, and therapeutic kits that comprise such ribozymes, vectors, virus particles, viral vectors, or host cells. These compositions preferably are formulated in pharmaceutically-acceptable excipients, suitable for ocular or subretinal administration to a mammalian eye. The compositions may also optionally further comprise one or more carriers, adjuvants, lipids, liposomes, lipid particles, nanoparticles, or microsphere formulations to facilitate administration to the affected eye. Such kits may include one or more of the compositions of the invention along with one or more devices for administering the therapeutic agents, as well as instructions for using the kit or its components in the therapy of the eye. For example, the kits of the invention may comprise a device such as a syringe or a needle, for delivering the compositions to the eye, retina, or subretinal space of a mammal.

In another important embodiment, the invention also provides a method for decreasing the amount of mRNA encoding a selected polypeptide in a retinal cell of a mammalian eye. This method generally involves providing to the eye a ribozyme composition in an amount and for a time effective to specifically cleave the mRNA in the cell, and thereby decrease the amount of mRNA in such a cell.

Such methods find particular utility in specifically cleaving an mRNA that encodes a polypeptide that causes a pathological condition in, or contributes to a disease, disorder, or dysfunction in a cell or a tissue of a mammalian eye. Examples of such conditions include, but are not limited to, retinal degeneration, retinitis, macular degeneration, and retinopathy, and particularly include conditions such as retinitis pigmentosa, autosomal dominant retinitis pigmentosa, autosomal recessive retinitis pigmentosa, macular degeneration, age-related macular degeneration, retinopathy, and diabetic retinopathy.

Likewise, the invention provides methods for decreasing the amount of a selected polypeptide in a cell or tissue of a mammalian eye. Such methods also generally involve providing or administering to an eye, a ribozyme construct of the present invention in an amount and for a time effective to specifically decrease the amount of the selected polypeptide in the cells or tissues of the eye. Similarly, the compositions of the invention may be used in methods for decreasing the amount of a selected polypeptide in the eye of a mammal suspected of having a pathological condition, and in methods for treating, decreasing the severity, or ameliorating the symptoms of a pathological condition that results from the expression of at least a first selected polypeptide in a cell or a tissue of a human eye. Examples of such symptoms include, but are not limited to, atrophic lesions of the eye, pigmented lesions of the eye, blindness, a reduction in central vision, a reduction in peripheral vision, and a reduction in total vision.

The invention also provides methods for decreasing the progression of such degenerative pathological conditions of a mammalian eye, and these methods typically comprise providing to such an eye one or more ribozymes, vectors, or viral particles of the invention, in an amount and for a time effective to decrease the progression of such degenerative pathological conditions.

A further aspect of the subject invention pertains to the reduction and/or elimination of pathological levels of proteins involved in endothelial cell nitric oxide (NO) regulation. This aspect of the subject invention provides materials and methods for the treatment and/or prevention of diabetic retinopathy. Increased inducible nitric oxide synthase (iNOS), enhanced vascular endothelial growth factor levels, and disruption of the blood retinal barrier has been identified in the retinas of BBZ/Wor diabetic rats compared to non-diabetic age-matched controls. Additionally, endothelial NOS (eNOS) has been identified in the plasmalemmal caveolae of retinal capillary endothelium from diabetic animals, and cytological evidence indicates translocation of the caveolae from the lumenal to the ablumenal surface of the endothelium. In high glucose environments, chronically increased NO activity results in endothelial cell dysfunction and impaired blood-retinal barrier integrity responsible for the development of diabetic retinopathy.

The ribozyme compositions of the present invention are preferably comprised within a vector suitable for delivery and expression in selected cells and tissues of the mammalian eye. For example, viral delivery vectors, and AAV-based vectors and virus particles are particularly preferred for delivery of the therapeutic catalytic molecules to the affected eye and the host cells and tissues comprised within the eye. These virus-vectored ribozyme molecules can be delivered to the target site by a variety of different methods, including for example, direct injection of the pharmaceutical compositions into the eye, the subretinal space, or the tissues immediately adjacent to the affected eye. These and other aspects of the present invention will be readily apparent to those of skill in the art having benefit of the present disclosure and the specific teachings disclosed hereinbelow:

### 3. BRIEF DESCRIPTION OF THE DRAWINGS

The following drawings form part of the present specification and are included to further demonstrate certain aspects of the present invention. The invention may be better understood by reference to the following description taken in conjunction with the accompanying drawings, in which like reference numerals identify like elements, and in which:

FIG. 1 shows adenosine, acting through its type A₂ receptor, can act to increase oxygen supply via two paths. During acute hypoxia, adenosine acts on smooth muscle cells, resulting in vasodilation (A_{2A}). With chronic ischemia, adenosine acts as an angiogenic agent by exerting a mitogenic effect on microvascular endothelial cells (in HREC, A_{2B}; see below). It is this latter effect that can be interfered with in an attempt to develop a pharmacological therapy for neovascular diseases. A distinct receptor subtype that mediates solely the mitogenic effect of adenosine would allow the targeting of a selective antagonist against that receptor subtype, without preventing the vasodilation mediated by the A_{2A} receptor;

FIG. 2A shows HREC proliferation after stimulation with NECA alone or in combination with a blocking antibody to VEGF. Open bars are results after 24 hr of exposure; filled bars are results after 48 hr. (*), significantly different from 10 μM NECA alone for the respective exposure time by ANOVA (p<0.05). Also shown are control cells exposed to VEGF alone or in combination with anti-VEGF to demonstrate the efficacy of the antibody;

FIG. 2B shows VEGF content in conditioned medium from HREC after stimulation with NECA in the presence or absence of sense or antisense oligonucleotides homologous to human A_{2B} adenosine receptor or to human VEGF. Assay duration was 48 hr. A_{2B} antisense treatment reduces the amount of VEGF protein secreted in response to NECA to levels equaling or exceeding the reduction evident by VEGF antisense treatment;

FIG. 3 shows NECA, at the concentrations indicated in the legends, induces a transient activation of ERK/MAPK in HREC that peaks at 5 min and desensitizes by 20 min after exposure. HREC were serum-starved for 24 hr and pre-treated for 20 min with 1 U/mL adenosine deaminase prior to adding NECA. Activated ERK/MAPK was visualized on Western blots by enhanced chemiluminescence using EC10 monoclonal antibody;

FIG. 4 shows the A₁-selective agonist CPA stimulates ERK/MAPK phosphorylation in HREC, however the A_{2A}-selective agonist CGS did not activate ERK/MAPK;

FIG. 5 shows HREC were pretreated for 30 min with the MEK inhibitor PD98059 or the PKA inhibitor H-89 and stimulated with NECA for 5 min. PD98059 inhibited ERK activation, while H-89 increased basal ERK activation. H-89 did not block NECA-stimulated ERK activation, suggesting that PKA is not involved in signaling from the adenosine receptor to ERK. The non-selective adenosine receptor antagonist XAC decreased ERK activation by high concentrations of NECA, but modestly increased ERK activation in control conditions and in response to 1 and 10 nM NECA. In contrast, PD98059 did not alter CREB, whereas both H-89 and XAC blocked NECA-induced CREB activation. These data indicate that NECA results in ERK activation independent of the cAMP response;

FIG. 6 shows both Enprofylline and JW V-108 antagonize activation of p42 and p44 ERK/MAP kinase by NECA. HRECs were serum-starved for 24 hr and pre-treated with adenosine deaminase (ADA, 1 U/mL) for 20 min, incubated with the antagonists in the presence of ADA for 10 min. NECA (1 nM-10 μM, 10 min) was used to activate ERK. ERK activation was analyzed by Western blot using the E10 monoclonal antibody, which recognizes the phosphorylated (active) form of the enzyme;

FIG. 7 shows a schematic representation (left) of the A_{2B} adenosine receptor ribozyme shows the nucleotide sequence of the recognition arms, as well as the complementary sequence (in red) of the synthetic target. Cleavage of this target by the ribozyme is shown in the autoradiogram (top right), demonstrating the cleavage kinetics. Band densities of cleaved vs. intact target were plotted as percent cleaved (bottom right). The A_{2B} receptor ribozyme cleaves nearly 90% of target in a 1:1 molar ratio by 60 min;

FIG. 8 shows A_{2B} adenosine receptor ribozyme reduces NECA-stimulated VEGF synthesis and cell proliferation in HREC. Cells were stimulated with 10 μmol/L NECA alone (♦), or NECA plus 1 μmol/L of either a mixed 37-mer oligoribonucleotide (sham, ▪) or A_{2B} ribozyme (▴). Both the amount of VEGF secreted into the medium (top) and the degree of proliferation (bottom) were decreased by the ribozyme, and not by the sham oligonucleotide control; and

FIG. 9 shows adenosine receptor antagonists reduce the degree of retinal neovascularization in the mouse pup model of oxygen-induced retinopathy. Daily IP injections of antagonists (30 mg/Kg body weight) resulted in a 54% to 70% reduction compared to untreated controls. The number of eyes examined for each condition was at least 16. *Significantly different (p<0.05) from uninjected.

FIG. 10 shows the number of neovascular nuclei counted per eye section for both the uninjected and AAV-IGF1R Rz1 injected eyes.

FIG. 11 shows a schematic illustration of a representative hairpin ribozyme molecule of the present invention.

FIG. 12 shows a schematic illustration of a representative hammerhead ribozyme molecule of the present invention. The sequences of the arms may bary, as shown in Tables 4-8).

### 4. DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The subject invention pertains to methods for achieving highly specific elimination and/or reduction of mutant and/or excess proteins associated with pathological conditions. Specifically exemplified herein is the use of ribozymes to treat and/or prevent diseases in the retina. In one aspect, the subject invention provides materials and methods which can be used to reduce or eliminate the symptoms of inherited eye disease caused by mutations in genes for retinal proteins.

The present invention utilizes the catalytic properties of ribozymes. Ribozymes are enzymes comprised of ribonucleic acid (RNA). In nature, ribozymes conduct a variety of reactions involving RNA, including cleavage and ligation of polynucleotide strands. The specificity of ribozymes is determined by base pairing (hydrogen bonding) between the targeting domain of the ribozyme and the substrate RNA. This specificity can be modified by altering the nucleotide sequence of the targeting domain. The catalytic domain of ribozymes, the part that actually performs the biochemical work, can also be changed in order to increase activity or stability of the ribozyme.

Ribozymes, if delivered as described herein to cells of the eye, and particularly to cells of the retina by a gene delivery vector, such as e.g., a specially designed virus, can provide a long-term, and even permanent treatment for a variety of retinal diseases, including, for example, retinitis pigmentosa, macular degeneration, or other pathological retina condition. Viral vectors, such as rAAV, are well known and readily available to those skilled in the art. Utilizing the techniques of the subject invention, ribozymes can be continuously produced in the retinal cells from a copy of the ribozyme integrated in the patient's DNA.

Ribozymes can also be used according to the subject invention as a partial treatment for recessive or semi-dominant genetic diseases of the eye as a supplement to gene replacement therapy. The delivery-expression materials and methods of the subject invention can be used to replace any gene responsible for recessive photoreceptor disease. Specific examples include the genes responsible for retinitis pigmentosa or macular degeneration. Additionally, ribozymes can be used according to the subject invention to treat RP-like disease resulting from the numerous known mutations in the rhodopsin gene. Examples of such mutations are well known to those skilled in the art. See, for example, Daiger et al., Behavioral Brain Sci., 18:452-67, 1995.

A further aspect of the current invention pertains to therapeutic strategies that can retard or block the effects of high glucose on progression of diabetic retinopathy. High glucose environments can result in chronically increased nitric oxide (NO) activity which leads to endothelial cell dysfunction and impaired blood retinal barrier integrity characteristic of diabetic retinopathy.

Reducing the synthesis of NOS using ribozymes can be used to retard or eliminate the damage to the blood retinal barrier. For example, ribozymes which reduce mRNA for VEGF, iNOS, or eNOS can be used. In specific embodiments, to inhibit the expression of iNOS and eNOS, hammerhead ribozymes that contain one long (46 nt) targeting arm 3′ to the catalytic domain and a short (5 nt) targeting sequence 5′ to the catalytic domain can be used. The long targeting arm permits rapid association with the target sequence. Keeping one arm short permits rapid dissociation of product necessary for catalytic turnover. Messenger RNA molecules have a complex pattern of intramolecular hydrogen bonds that reduce the portion of the molecule available for ribozyme attack. Sites in the iNOS and eNOS mRNAs accessible to ribozyme binding can be determined using synthetic transcripts of iNOS and eNOS cDNA clones. Ribozyme cleavage can be tested on short oligonucleotides identical to sequences of accessible regions containing hammerhead target sites. The most active ribozymes can then be tested on synthetic transcripts of the entire cDNA clone and on total mRNA extracted from endothelial cells to identify the most preferred ribozymes.

Genes encoding ribozymes can be cloned in the AAV vector or other suitable vector. High-potency ribozymes that cleave eNOS, iNOS, and/or VEGF mRNA can be constructed by those skilled in the art having the benefit of the instant disclosure. Delivering these to retinal endothelial cells can be done to reduce expression of iNOS, eNOS, or VEGF and, ultimately, to reduce the production of nitric oxide. Reduction of NO production will, in turn, reduce or delay retinal permeability dysfunction.

4.1 Insulin-Like Growth Factor-I

IGF-I, together with platelet-derived growth factor, accounts for most of the growth-promoting activity of serum and is recognized as one of the progression factors that prompt “competence factor”-primed cells to proceed through the prereplicative phase of the cell cycle, G₁ (Clemmons, 1992). Cloning the IGF-I receptor definitively demonstrated that activation of an overexpressed IGF-I receptor could initiate mitogenesis and promote ligand-dependent neoplastic transformation. IGF-I action is tightly regulated by a series of IGF binding proteins (IGFBPs) (Guenette et al., 1994; Grant and King, 1995).

While VEGF is currently viewed as the major effector for retinal neovascularization (Aiello et al., 1994; Robinson et al., 1996), recent studies further point to a pivotal role for IGF-1 in retinal neovascularization. IGF-I receptors are present on retinal microvascular cells and these cells respond to IGF-I with a five-fold increase in DNA synthesis (King et al., 1985; Grant et al., 1993a). IGF-I promotes chemotaxis (migration) of human and bovine retinal endothelial cells in a concentration dependent manner (Grant et al., 1987). IGF-I modulates protease expression (Grant et al., 1993b) and acts as a survival factor for the retinal microvessel cells. In later stages of proliferative diabetic retinopathy, it can induce retinal angiogenesis and is expressed by several retinal cell types in response to VEGF exposure (Punglia et al., 1997). Data have demonstrated that VEGF induces IGF-I and bFGF production by HRECs.

Antibodies to IGF-I receptor, antisense strategies against IGF-I and IGF-I receptor, and dominant negative IGF-I Rc mutants all reduce cell survival and promote cell death (Beck et al., 1995). Conversely, overexpression of IGF-I receptor enhances cell survival in response to death signals (Dunn et al., 1997).

Altered IGF-I levels are clinically meaningful in diabetes and may be important in permitting apoptosis in response to the diabetic state. The serum level of IGF-I is reduced acutely in both clinical and experimental diabetes despite higher than normal growth hormone levels because hepatic IGF-I production requires the presence of portal insulin (Sonksen et al., 1993). In streptozotocin-treated rats, there is a decrease in serum IGF-I levels and a reduction in IGF-I mRNA in liver, kidney, lung and heart during the first month of diabetes, in part due to a loss of portal insulin (Yang et al., 1990). The observations that IGF-I mimics insulin's metabolic effects suggested that IGF-I could be used therapeutically to restore euglycemia. However, clinical trials with recombinant human (rh) IGF-I in patients with both insulin dependent diabetes mellitus (IDDM) and non-insulin dependent diabetes mellitus (NIDDM) were halted due to progression of retinopathy, with optic nerve neovascularization and other microvascular complications (Kolaczynski and Caro, 1994; Langford and Miell, 1993; Cusi and DeFronzo, 1995). Doses of rhIGF-I that are required to improve hyperglycemia may be limited by adverse effects and several investigators caution that rhIGF-I treatment could accelerate progression of diabetic retinopathy (Kolaczynski and Caro, 1994; Langford and Miell, 1993; Cusi and DeFronzo, 1995). Results of a recently published clinical trial in patients with severe nonproliferative diabetic retinopathy or “non high risk” proliferative diabetic retinopathy found that Octreotide, a growth hormone and IGF-I antagonist, delayed the need for laser photocoagulation.

The local tissue levels of IGF-I are probably as relevant as serum levels to the initiation of diabetic complications (Grant and King, 1995). Several clinical studies support a role for IGF-I in development of retinal neovascularization (Merimee et al, 1983; Hyer et al., 1989; Dills et al., 1991). Studies have demonstrated a three-fold increase of IGF-I in the vitreous of diabetics with proliferative diabetic retinopathy compared to nondiabetic individuals. These findings were independently confirmed (Meyer-Schwickerath et al., 1993).

This dysregulation of IGF-I may result in apoptosis as seen in nonproliferative diabetic retinopathy and proliferation as seen later in proliferative retinopathy. These studies emphasize the importance of the appropriate amount of IGF-I, since too little results in apoptosis and acellular capillaries, too much promotes aberrant endothelial proliferation, and the appropriate amount ensures endothelial cell survival in the retina.

4.2 Adenosine and Angiogenesis

Retinal ischemia and abnormal angiogenesis occur not only in PDR, but also in retinopathy of prematurity (ROP) and in age-related macular degeneration. Substantial evidence supports a role for adenosine in promoting angiogenesis (Dusseau and Hutchins, 1988; Adair et al., 1989). Studies suggest that adenosine can act as a mitogen in endothelial cells derived from various vascular beds (Sexl et al., 1995; Grant et al., 1999) to increase cell number, DNA synthesis (Ethier et al., 1993), cell migration and vascularity (Dusseau et al., 1986). Endothelial cells are known to have a very active adenosine metabolism, characterized by a large capacity for uptake and release of the nucleoside (Nees et al., 1985). Adenosine can stimulate endothelial cells to alter their pattern of gene expression (Takagi et al., 1996a). High levels of adenosine are associated with areas of vasculogenesis in the normal neonatal dog retina as well as sites of angiogenesis in the canine model of oxygen and induced retinopath (Taomoto et al., 2000; Lutty et al., 2000). Data show that the adenosine analogue NECA increases vascular endothelial cell growth factor (VEGF) mRNA in human retinal endothelial cells (HREC) (Grant et al., 1999). In addition to mediating VEGF expression, adenosine has a synergistic effect with VEGF on retinal endothelial cell migration and capillary morphogenesis in vitro (Lutty et al., 1998).

Adenosine is a critical mediator of blood flow changes in response to ischemia. It is a significant component of the retina's compensatory hyperemic response to ischemia, hypoxia, and hypoglycemia (Rego et al., 1996). Increasing endogenous adenosine concentrations may be useful in ameliorating post-ischemic hypoperfusion. Current evidence suggests that adenosine is a vital component of the endogenous retinal response to substrate deprivation. Adenosine is a potent vasodilator. It has been appreciated that vasodilators increase the growth of endothelial cells while vasoconstrictors increase the growth of smooth muscle cells (Brown and Jampol, 1996). In the retinal microvasculature, adenosine and adenosine analogues cause concentration-dependent vasodilation (Gidday and Park, 1993). The vasodilatory response of retinal arterioles to hypoxia in newborn piglets is attenuated by the nonselective adenosine receptor antagonist, 8SPT. Likewise, 8SPT inhibits retinal arteriolar vasodilation induced by systemic hypotension, whereas inhibiting adenosine uptake with S(4-nitrobenzyl)-6-thioinosine (NBTI) potentiates, arteriolar dilation. Altogether, these observations strongly support a role for endogenously released adenosine as a key mediator of blood flow during conditions of reduced O₂ supply (Gidday and Park, 1993).

In bovine retinal endothelial cells and pericytes, adenosine receptor inhibition reduces the induction of VEGF mRNA and protein expression when cells are exposed to hypoxic conditions (Takagi et al., 1996b). Hypoxia-induced increases in VEGF mRNA were inhibited by adenosine deaminase, an enzyme that degrades adenosine to inosine, which does not activate adenosine receptor. The adenosine receptor antagonist, 8-phenyltheophylline (Ethier et al., 1993), can block the proliferative effect of adenosine. Theophylline and 3,7-dimethyl-1-propylxanthine (DMPX), nonselective adenosine receptor antagonists, also inhibited VEGF mRNA induction following hypoxia (Hashimoto et al., 1994). The weak adenosine antagonist theobromine caused significant inhibition of angiogenic activity of ovarian cancer cells and decreased VEGF production in vitro in these cells (Barcz et al., 1998).

4.3 Adenosine Receptors

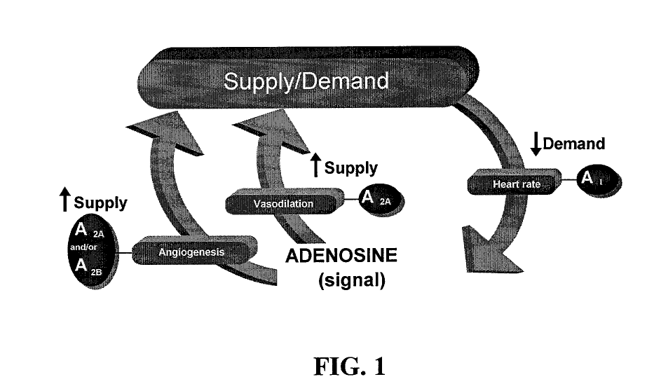

Adenosine can interact with at least four subtypes of G-protein coupled receptors, termed A₁, A_{2A}, A_{2B} and A₃ (Shryock and Belardinelli, 1997). These receptors are encoded by distinct genes and can be differentiated based on their affinities for adenosine agonists and antagonists (Fredholm et al., 1994). A₁ and A₃ adenosine receptors interact with pertussis toxin-sensitive G proteins of the G₁ and G₀ type to inhibit adenylate cyclase, whereas A_{2A} (high affinity) and A_{2B} (low affinity) adenosine receptors stimulate adenylate cyclase via G, (Fredholm et al., 1994). In most cell types and organ systems, adenosine activates A₁ adenosine receptors to decrease work (decrease O₂ demand), whereas A₂ adenosine receptors increase O₂ supply (Shryock and Belardinelli, 1997). Thus, adenosine, by increasing O₂ supply (activation of A₂ adenosine receptor) and by decreasing O₂ demand (activation of A₁ adenosine receptor), is an ideal candidate to rectify imbalances between O₂ supply and demand. This has led to the concept that adenosine is a protective metabolite (Shryock and Belardinelli, 1997 (FIG. 1).

Studies suggest that adenosine acting via the A_{2B} adenosine receptor could promote angiogenesis. Thus, A₂ adenosine receptors mediate short term increases in O₂ supply by increasing blood flow and long term by increasing vascularity (FIG. 1). Adenosine increases cAMP production and the consequences of adenylate cyclase stimulation in endothelial cells include cell shape changes, and changes in junctional permeability in addition to angiogenesis (Stelzner et al., 1989; Tuder et al., 1990). Signaling pathways mediating the mitogenic action of adenosine include mitogen activated protein kinase (MAPK) (Sexl et al., 1997) and protein kinase A (PKA) (Takagi et al., 1996b).

Investigators have identified the A_{2A} receptor as the mediator of adenosine's actions in different species. Takagi et al. (1996a) reported that endogenously released adenosine stimulates VEGF gene expression in bovine retinal endothelial cells and pericytes through stimulation of A_{2A} adenosine receptor. A₂ receptors are associated with vessels. Lutty demonstrated that A_{2A} receptor localized to the edge of the developing vasculature in canine retina. Taomoto and coworkers also demonstrated high levels of A_{2A} receptor immunoreactivity in immature intravitreal neovascular formations in the canine oxygen-induced retinopathy model (Taomoto et al., 2000).

4.4 Hypoxia Inducible Factors

Hypoxia and its subsequent tissue ischemia induce a significant increase in extracellular adenosine and hypoxanthine and to a lesser extent inosine. Local hypoglycemia, often associated with tissue hypoxia, also induces adenosine and hypoxanthine formation and release. Hypoxia also activates the expression of a number of genes, principally by the stabilization of members of the basic helical-loop-helix (bHLH)—PAS family of transcription factors that bind to the hypoxia response element (HRE), a consensus DNA sequence, the hypoxia response element (HRE). Studies of the erythropoietin (Epo) gene led to the identification of hypoxia inducible factor (HIF-1). HIF-1 is a transcription factor activated by hypoxia. HIF-1 is composed of two subunits HIF-1α and HIF-1β, both bHLH-PAS proteins. HIF-1α protein expression is rapidly induced by hypoxia and the magnitude of the response is inversely related to the cellular O₂ concentration. Dimerization with HIF-1β induces a conformational change in HIF-1α, possibly mediated by HSP90, which is required for high affinity binding to DNA. Hypoxia response elements (HREs) containing functionally essential HIF-1 binding sites were identified in genes encoding VEGF, glucose transport 1 and the glycolytic enzymes, aldolase A, enolase, lactate dehydrogenase A and phosphoglycerate kinase 1. HIF-1 has also been shown to activate transcription of genes encoding inducible nitric oxide synthase and heme oxygenase 1 which are responsible for the synthesis of the vasoactive molecules NO and CO, respectively, and transferring, which, like Epo, is essential for erythropoiesis.

The known target genes demonstrate that HIF-1 facilitates both increased O₂ delivery, by promoting erythropoiesis, angiogenesis, and vasodilation, and decreased O₂ utilization, by participating in the transition from oxidative phosphorylation to glycolysis as a means of generating ATP. The HIF-1 activation transduction pathway is poorly understood. Extracellular regulated kinases (ERK), members of the MAPK family of kinases, are activated in hypoxia. Minet and coworkers demonstrated that in human microvascular endothelial cells, ERK kinases are activated during hypoxia. Using dominant negative mutants, they showed that ERK is needed for hypoxia induced HIF-1 transactivation activity and that HIF-1α is phosphorylated in hypoxia by an ERK-dependent pathway (Minet et al., 2000). It was shown that exposure of HREC to adenosine agonists results in activation of ERK. Adenosine is released during hypoxia, thus during hypoxia adenosine may be mediating the phosphorylation of HIF by activation of ERK.

4.5 Progenitor Endothelial Cells Incorporate into Sites of Active Angiogenesis

Vasculogenesis is the in situ differentiation of mesodermal precursors to angioblasts that differentiate into endothelial cells to form the primitive capillary network. Vasculogenesis is limited to early embryogenesis and is believed not to occur in the adult. By contrast, angiogenesis is the sprouting of new capillaries from pre-existing blood vessels and occurs in late embryogenesis and postnatal life. The basic mechanisms underlying vasculogenesis and angiogenesis are at present unclear. Human stem cells from peripheral blood can differentiate into endothelial cells. A number of reports have demonstrated the presence of circulating endothelial cells. Asahara et al. showed that CD34⁺ cells derived from peripheral circulation could form endothelial colonies (Asahara et al., 1997). These were identified by their ability to incorporate acetylated LDL, express PCAM and Tie-2 receptors and produce nitric oxide following VEGF stimulation. CD34 is a marker for hematopoietic progenitor cells that give rise to all blood cells and is found on all endothelial cells in the adult and developing embryo. Thus, the hemangioblast apparently gives rise to both the hematopoietic cells and vascular cells during embryogenesis.

Putative angioblasts were isolated from the leukocyte fraction of peripheral blood and contributed to angiogenic blood vessel formation in a rabbit model of hindlimb ischemia. In these elegant studies by Asahara et al., human CD34⁺ cells were administered to C57BL/6J 129/SV background athymic nude mice. Two days after creating hindlimb ischemia by excising one femoral artery, the mice were injected with 1,1′-dioctadecyl-3,3,3′,3′-tetramethylindocarbocyanine perchlorate (DiI)-labeled human CD34⁺ cells into their tail veins. One to six weeks later histological examination revealed numerous DiI-labeled cells in the neovascularized ischemic hindlimb (Asahara et al., 1997). Nearly all labeled cells appeared integrated into capillary vessel walls. DiI-labeled cells consistently colocalized with cells immunostaining for CD31 and Tie-2, endothelial cell markers. No labeled cells were found in uninjured limbs. Similarly DiI-labeled positive for the VEGF receptor Flk⁺ were administered to C57BL/6J 129/SV background athymic nude mice after creating hindlimb ischemia, with results similar to CD34⁺ cells. (Asahara et al., 1997). These results support that circulating cells that express CD34 and Flk contribute to neoangiogenesis in adult animals, consistent with angiogenesis, a paradigm otherwise restricted to embryogenesis (Flamme and Risau, 1992).

Determining the origin of endothelial cells that form preretinal neovascularization is critical to developing nondestructive therapies to treat the condition. It is feasible to imagine that the diabetic individual may have an increased number of circulating endothelial precursor cells (EPCs) due to the abnormal hormonal and metabolic milieu associated with the diabetic state. Increased serum levels of the growth factors IGF-I and VEGF as well as of the cytokine TNF-α have been found in diabetics with proliferative diabetic retinopathy (Grant et al., 1986; Limb et al., 1996). These growth factors and cytokines could increase the number of circulating bone marrow (BM)-derived EPCs (Bikfalvi and Han, 1994). EPCs may home to tissue stroma in the eye for purposes of providing maintenance reservoirs of EPCs, analogous to satellite myoblasts and fibroblasts (Asahara et al., 1997; Bikfalvi and Han, 1994). Local growth factors released in response to adenosine could also increase the expression of as yet unidentified cell adhesion molecules and stimulate supportive stromal cells that would likely contribute to enhanced homing of circulating EPCs to the retina. EPCs may further differentiate and/or incorporate into foci of neovascularization.

4.6 Gene Therapy for Diabetic Retinopathy

Recombinant adeno-associated virus (rAAV) vectors have been used successfully in long-term gene delivery to the retina (Flannery et al., 1997; Bennett et al., 1997), the lung (Flotte et al., 1993), muscle (Kessler et al., 1996; Xiao and Samulski, 1996), brain (Klein et al., 1998; Xiao et al., 1997), spinal cord (Peel et al., 1997), liver (Snyder et al., 1997) and blood vessels (Rolling et al., 1997). In particular, AAV infects vascular endothelial cells in vivo (Gnatenko et al., 1997; Lynch et al., 1997). A single intravitreal injection of AAV expressing marker proteins (β-galactosidase or gfp) was able to genetically transduce a variety of cell types in the guinea pig eye, including blood vessels, for up to one year post injection with no evidence of inflammation or other abnormalities (Guy et al., 1999). Unlike adenovirus vectors used in gene therapy trials, AAV does not cause inflammation and does not provoke a cell mediated immune response (Bennett et al., 1997; Bennett et al., 1999). Unlike retroviral vectors, AAV is able to infect non-cycling cells, such as vascular endothelial cells. Retroviral vectors have been used for dividing endothelial cells in culture, but not for non-dividing cells in vivo.

A low therapeutic index (ratio of toxic dose to therapeutic dose) is important for gene-based therapies, and one approach to achieve this has been transcriptional targeting through use of tissue-specific regulatory elements. For example, we have employed the rhodopsin promoter to achieve photoreceptor-specific expression of ribozymes in rats (Lewin et al., 1998). A more versatile approach might be to use a regulatory element that is controlled by a condition common to a broad range of diseases, i.e., ischemia. Ischemia is characteristic of a number of pathologies ranging from vascular occlusion to cancer. Consequently, several research groups are developing vectors for gene delivery that employ regulation by the hypoxia response element (HRE). These cis-acting elements have been identified as enhancer elements in the 5′ or 3′ flanking region of a variety of hypoxia-regulated genes, but have been best characterized in the context of the genes for erythropoietin, VEGF and the glycolytic enzyme phosphoglycerate kinase 1 (PGK1). A hypoxia-regulated element from the PGK1 gene showed a 50-fold induction upstream of a minimal SV40 promoter in the context of either an adenoviral vector (Binley et al., 1999) or a retroviral vector (Boast et al., 1999). These levels of expression of marker genes (β-galactosidase and luciferase, respectively) were equivalent to the unregulated expression of the same genes in the same cultured cells directed by the cytomegalovirus (CMV) immediate early promoter. Hypoxia-regulated vectors may have utility for restricting the delivery of therapeutic proteins to ischemic sites. The fact that progenitor endothelial cells home to sites of ischemia suggests potential utility as autologous vectors for gene therapy. For antiangiogenic therapies, CD34⁺ cells could be transfected with angiogenesis inhibitors.

4.7 Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 (specifically incorporated herein by reference) reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

Six basic varieties of naturally occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al., 1992). Thus, the specificity of action of a ribozyme is greater than that of an antisense oligonucleotide binding the same RNA site.

The enzymatic nucleic acid molecule may be formed in a hammerhead, hairpin, a hepatitis 5 virus, group I intron or RNaseP RNA (in association with an RNA guide sequence) or Neurospora VS RNA motif. Examples of hammerhead motifs are described by Rossi et al. (1992). Examples of hairpin motifs are described by Hampel et al. (Eur. Pat. Appl. Publ. No. EP 0360257), Hampel and Tritz (1989), Hampel et al. (1990) and U.S. Pat. No. 5,631,359 (specifically incorporated herein by reference). An example of the hepatitis 8 virus motif is described by Perrotta and Been (1992); an example of the RNaseP motif is described by Guerrier-Takada et al. (1983); Neurospora VS RNA ribozyme motif is described by Collins (Saville and Collins, 1990; Saville and Collins, 1991; Collins and Olive, 1993); and an example of the Group I intron is described in U.S. Pat. No. 4,987,071 (specifically incorporated herein by reference). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

In certain embodiments, it may be important to produce enzymatic cleaving agents that exhibit a high degree of specificity for the RNA of a desired target, such as one of the sequences disclosed herein. The enzymatic nucleic acid molecule is preferably targeted to a highly conserved sequence region of a target mRNA. Such enzymatic nucleic acid molecules can be delivered exogenously to specific cells as required, although in preferred embodiments the ribozymes are expressed from DNA or RNA vectors that are delivered to specific cells.

Small enzymatic nucleic acid motifs (e.g., of the hammerhead or the hairpin structure) may also be used for exogenous delivery. The simple structure of these molecules increases the ability of the enzymatic nucleic acid to invade targeted regions of the mRNA structure. Alternatively, catalytic RNA molecules can be expressed within cells from eukaryotic promoters (e.g. Scanlon et al., 1991; Kashani-Sabet et al., 1992; Dropulic et al., 1992; Weerasinghe et al., 1991; Ojwang et al., 1992; Chen et al., 1992; Sarver et al., 1990). Those skilled in the art realize that any ribozyme can be expressed in eukaryotic cells from the appropriate DNA vector. The activity of such ribozymes can be augmented by their release from the primary transcript by a second ribozyme (Int. Pat. Appl. Publ. No. WO 93/23569, and Int. Pat. Appl. Publ. No. WO 94/02595, both hereby incorporated by reference; Ohkawa et al., 1992; Taira et al., 1991; and Ventura et al., 1993).

Ribozymes may be added directly, or can be complexed with cationic lipids, lipid complexes, packaged within liposomes, or otherwise delivered to target cells. The RNA or RNA complexes can be locally administered to relevant tissues ex vivo, or in vivo through injection, aerosol inhalation, infusion pump or stent, with or without their incorporation in biopolymers.

Ribozymes may be designed as described in Int. Pat. Appl. Publ. No. WO 93/23569 and Int. Pat. Appl. Publ. No. WO 94/02595 (each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Hammerhead or hairpin ribozymes may be individually analyzed by computer folding (Jaeger et al., 1989) to assess whether the ribozyme sequences fold into the appropriate secondary structure, as described herein. Those ribozymes with unfavorable intramolecular interactions between the binding arms and the catalytic core are eliminated from consideration. Varying binding arm lengths can be chosen to optimize activity. Generally, at least 5 or so bases on each arm are able to bind to, or otherwise interact with, the target RNA.

Ribozymes of the hammerhead or hairpin motif may be designed to anneal to various sites in the mRNA message, and can be chemically synthesized. The method of synthesis used follows the procedure for normal RNA synthesis as described in Usman et al. (1987) and in Scaringe et al. (1990) and makes use of common nucleic acid protecting and coupling groups, such as dimethoxytrityl at the 5′-end, and phosphoramidites at the 3′-end. Average stepwise coupling yields are typically >98%. Hairpin ribozymes may be synthesized in two parts and annealed to reconstruct an active ribozyme (Chowrira and Burke, 1992). Ribozymes may be modified extensively to enhance stability by modification with nuclease resistant groups, for example, 2′-amino, 2′-C-allyl, 2′-flouro, 2′-o-methyl, 2′-H (for a review see e.g., Usman and Cedergren, 1992). Ribozymes may be purified by gel electrophoresis using general methods or by high-pressure liquid chromatography and resuspended in water.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Perrault et al, 1990; Pieken et al., 1991; Usman and Cedergren, 1992; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

A preferred means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol 1), RNA polymerase II (pol II), or RNA polymerase III (pol 111). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells (Elroy-Stein and Moss, 1990; Gao and Huang, 1993; Lieber et al., 1993; Zhou et al., 1990). Ribozymes expressed from such promoters can function in mammalian cells (Kashani-Sabet et al., 1992; Ojwang et al., 1992; Chen et al., 1992; Yu et al., 1993; L'Huillier et al., 1992; Lisziewicz et al., 1993). Although incorporation of the present ribozyme constructs into adeno-associated viral vectors is preferred, such transcription units can be incorporated into a variety of vectors for introduction into mammalian cells, including but not restricted to, plasmid DNA vectors, other viral DNA vectors (such as adenovirus vectors), or viral RNA vectors (such as retroviral, semliki forest virus, sindbis virus vectors).

Sullivan et al. (Int. Pat. Appl. Publ. No. WO 94/02595) describes general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraocular, retinal, subretinal, intraperitoneal and/or intrathecal delivery. More detailed descriptions of ribozyme and rAAV vector delivery and administration are provided in Int. Pat. Appl. Publ. No. WO 94/02595 and Int. Pat. Appl. Publ. No. WO 93/23569, each specifically incorporated herein by reference.

Ribozymes and the AAV vectored-constructs of the present invention may be used to inhibit gene expression and define the role (essentially) of specified gene products in the progression of one or more retinal diseases and/or disorders. In this manner, other genetic targets may be defined as important mediators of the disease. These studies lead to better treatment of the disease progression by affording the possibility of combination therapies (e.g., multiple ribozymes targeted to different genes, ribozymes coupled with known small molecule inhibitors, or intermittent treatment with combinations of ribozymes and/or other chemical or biological molecules).

4.8 Promoters and Enhancers

Recombinant vectors form important aspects of the present invention. The term “expression vector or construct” means any type of genetic construct containing a nucleic acid in which part or all of the nucleic acid encoding sequence is capable of being transcribed. In preferred embodiments, expression only includes transcription of the nucleic acid, for example, to generate ribozyme constructs.

Particularly useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under the control” or “under the transcriptional control” means that the promoter is in the correct location and orientation in relation to the nucleic acid to control RNA polymerase initiation and expression of the gene.

In preferred embodiments, it is contemplated that certain advantages will be gained by positioning the coding DNA segment under the control of a recombinant, or heterologous, promoter. As used herein, a recombinant or heterologous promoter is intended to refer to a promoter that is not normally associated with a ribozyme construct in its natural environment. Such promoters may include promoters normally associated with other genes, and/or promoters isolated from any other bacterial, viral, eukaryotic, or mammalian cell.

Naturally, it will be important to employ a promoter that effectively directs the expression of the DNA segment in the cell type, organism, or even animal, chosen for expression. The use of promoter and cell type combinations for protein expression is generally known to those of skill in the art of molecular biology; for example, see Sambrook et al. (1989), incorporated herein by reference. The promoters employed may be constitutive, or inducible, and can be used under the appropriate conditions to direct high-level expression of the introduced DNA segment.

At least one module in a promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a nucleic acid is not believed to be critical, so long as it is capable of expressing the nucleic acid in the targeted cell. Thus, where a human cell is targeted, it is preferable to position the nucleic acid coding region adjacent to and under the control of a promoter that is capable of being expressed in a human cell. Generally speaking, such a promoter might include either a human or viral promoter, such as a CMV or an HSV promoter. In certain aspects of the invention, tetracycline controlled promoters are contemplated.

In various other embodiments, the human cytomegalovirus (CMV) immediate early gene promoter, the SV40 early promoter and the Rous sarcoma virus long terminal repeat can be used to obtain high-level expression of transgenes. The use of other viral or mammalian cellular or bacterial phage promoters that are well known in the art to achieve expression of a transgene is contemplated as well, provided that the levels of expression are sufficient for a given purpose. Tables 1 and 2 below list several elements/promoters that may be employed, in the context of the present invention, to regulate the expression of the present ribozyme constructs. This list is not intended to be exhaustive of all the possible elements involved in the promotion of transgene expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally any promoter/enhancer combination (as per the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

TABLE 1
PROMOTER AND ENHANCER ELEMENTS

| PROMOTER/ENHANCER | REFERENCES |
|---|---|
| Immunoglobulin Heavy Chain | Banerji et al., 1983; Gilles et al., 1983; Grosschedl and Baltimore, |
| | 1985; Atchinson and Perry, 1986, 1987; Imler et al., 1987; |
| | Weinberger et al., 1984; Kiledjian et al., 1988; Porton et al., 1990 |
| Immunoglobulin Light Chain | Queen and Baltimore, 1983; Picard and Schaffner, 1984 |
| T-Cell Receptor | Luria et al., 1987; Winoto and Baltimore, 1989; Redondo et al., |
| | 1990 |
| HLA DQ a and DQ β | Sullivan and Peterlin, 1987 |
| β-Interferon | Goodbourn et al., 1986; Fujita et al., 1987; Goodbourn and |
| | Maniatis, 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase | Jaynes et al., 1988; Horlick and Benfield, 1989; Johnson et al., 1989 |
| Prealbumin (Transthyretin) | Costa et al., 1988 |
| Elastase I | Ornitz et al., 1987 |
| Metallothionein | Karin et al., 1987; Culotta and Hamer, 1989 |
| Collagenase | Pinkert et al., 1987; Angel et al., 1987 |
| Albumin Gene | Pinkert et al., 1987; Tronche et al., 1989, 1990 |
| α-Fetoprotein | Godbout et al., 1988; Campere and Tilghman, 1989 |
| t-Globin | Bodine and Ley, 1987; Perez-Stable and Constantini, 1990 |
| β-Globin | Trudel and Constantini, 1987 |
| e-fos | Cohen et al., 1987 |
| c-HA-ras | Treisman, 1986; Deschamps et al., 1985 |
| Insulin | Edlund et al., 1985 |
| Neural Cell Adhesion Molecule | Hirsh et al., 1990 |
| (NCAM) | |
| α_{1-Antitrypain} | Latimer et al., 1990 |
| H2B (TH2B) Histone | Hwang et al., 1990 |
| Mouse or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A | Edbrooke et al., 1989 |
| (SAA) | |
| Troponin I (TN I) | Yutzey et al., 1989 |
| Platelet-Derived Growth Factor | Pech et al., 1989 |
| Duchenne Muscular Dystrophy | Klamut et al., 1990 |
| SV40 | Banerji et al., 1981; Moreau et al., 1981; Sleigh and Lockett, 1985; |
| | Firak and Subramanian, 1986; Herr and Clarke, 1986; Imbra and |
| | Karin, 1986; Kadesch and Berg, 1986; Wang and Calame, 1986; |
| | Ondek et al., 1987; Kuhl et al., 1987; Schaffner et al., 1988 |
| Polyoma | Swartzendruber and Lehman, 1975; Vasseur et al., 1980; Katinka |
| | et al., 1980, 1981; Tyndall et al., 1981; Dandolo et al., 1983; de |
| | Villiers et al., 1984; Hen et al., 1986; Satake et al., 1988; Campbell |
| | and Villarreal, 1988 |
| Retroviruses | Kriegler and Botchan, 1982, 1983; Levinson et al., 1982; Kriegler |
| | et al., 1983, 1984a, b, 1988; Bosze et al., 1986; Miksicek et al., |
| | 1986; Celander and Haseltine, 1987; Thiesen et al., 1988; Celander |
| | et al., 1988; Choi et al., 1988; Reisman and Rotter, 1989 |
| Papilloma Virus | Campo et al., 1983; Lusky et al., 1983; Spandidos and Wilkie, 1983; |
| | Spalholz et al., 1985; Lusky and Botchan, 1986; Cripe et al., 1987; |
| | Gloss et al., 1987; Hirochika et al., 1987; Stephens and Hentschel, |
| | 1987; Glue et al., 1988 |
| Hepatitis B Virus | Bulla and Siddiqui, 1986; Jameel and Siddiqui, 1986; Shaul and |
| | Ben-Levy, 1987; Spandau and Lee, 1988; Vannice and Levinson, |
| | 1988 |
| Human Immunodeficiency Virus | Muesing et al., 1987; Hauber and Cullan, 1988; Jakobovits et al., |
| | 1988; Feng and Holland, 1988; Takebe et al., 1988; Rosen et al., |
| | 1988; Berkhout et al., 1989; Laspia et al., 1989; Sharp and |
| | Marciniak, 1989; Braddock et al., 1989 |
| Cytomegalovirus | Weber et al., 1984; Boshart et al., 1985; Foecking and Hofstetter, |
| | 1986 |
| Gibbon Ape Leukemia Virus | Holbrook et al., 1987; Quinn et al., 1989 |

TABLE 2
INDUCIBLE ELEMENTS

| ELEMENT | INDUCER | REFERENCES |
|---|---|---|
| MT II | Phorbol Ester (TFA) | Palmiter et al., 1982; Haslinger and Karin, 1985; |
| | Heavy metals | Searle et al., 1985; Stuart et al., 1985; Imagawa |
| | | et al., 1987, Karin et al., 1987; Angel et al., |
| | | 1987b; McNeall et al., 1989 |
| MMTV (mouse mammary | Glucocorticoids | Huang et al., 1981; Lee et al., 1981; Majors and |
| tumor virus) | | Varmus, 1983; Chandler et al., 1983; Lee et al., |
| | | 1984; Ponta et al., 1985; Sakai et al., 1988 |
| β-Interferon | poly(rI)x | Tavernier et al., 1983 |
| | poly(rc) | |
| Adenovirus 5 E2 | Ela | Imperiale and Nevins, 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | Ela, SV40 Large T | Taylor et al., 1989; Taylor and Kingston, |
| | Antigen | 1990a, b |
| Proliferin | Phorbol Ester-TPA | Mordacq and Linzer, 1989 |
| Tumor Necrosis Factor | FMA | Hensel et al., 1989 |
| Thyroid Stimulating | Thyroid Hormone | Chatterjee et al., 1989 |
| Hormone a Gene | | |

As used herein, the terms “engineered” and “recombinant” cells are intended to refer to a cell into which an exogenous DNA segment, such as DNA segment that leads to the transcription of a ribozyme, has been introduced. Therefore, engineered cells are distinguishable from naturally occurring cells, which do not contain a recombinantly introduced exogenous DNA segment. Engineered cells are thus cells having DNA segment introduced through the hand of man.

To express a ribozyme in accordance with the present invention one would prepare an expression vector that comprises a ribozyme-encoding nucleic acid under the control of one or more promoters. To bring a sequence “under the control of” a promoter, one positions the 5′ end of the transcription initiation site of the transcriptional reading frame generally between about 1 and about 50 nucleotides “downstream” of (ie., 3′ of) the chosen promoter. The “upstream” (ie., 5′) promoter stimulates transcription of the DNA and promotes expression of the encoded ribozyme. This is an exemplary meaning of “recombinant expression” when used in the context of the present invention.

4.9 Adeno-Associated Virus (AAV)

Adeno-associated virus (AAV) is particularly attractive for gene transfer because it does not induce any pathogenic response and can integrate into the host cellular chromosome (Kotin et al., 1990). The AAV terminal repeats (TRs) are the only essential cis-components for the chromosomal integration (Muzyczka and McLaughin, 1988). These TRs are reported to have promoter activity (Flotte et al., 1993). They may promote efficient gene transfer from the cytoplasm to the nucleus or increase the stability of plasmid DNA and enable longer-lasting gene expression (Bartlett et al., 1996). Studies using recombinant plasmid DNAs containing AAV TRs have attracted considerable interest. AAV-based plasmids have been shown to drive higher and longer transgene expression than the identical plasmids lacking the TRs of AAV in most cell types (Philip et al., 1994; Shafron et al., 1998; Wang et al., 1999).

AAV (Ridgeway, 1988; Hermonat and Muzyczka, 1984) is a parovirus, discovered as a contamination of adenoviral stocks. It is a ubiquitous virus (antibodies are present in 85% of the US human population) that has not been linked to any disease. It is also classified as a dependovirus, because its replication is dependent on the presence of a helper virus, such as adenovirus. Five serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter (Muzyczka and McLaughlin, 1988).

The AAV DNA is approximately 4.7 kilobases long. It contains two open reading frames and is flanked by two ITRs. There are two major genes in the AAV genome: rep and cap. The rep gene encodes a protein responsible for viral replications, whereas the cap gene encodes the capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three viral promoters have been identified and named p5, p19, and p40, according to their map position. Transcription from p5 and p19 results in production of rep proteins, and transcription from p40 produces the capsid proteins (Hermonat and Muzyczka, 1984).

There are several factors that prompted researchers to study the possibility of using rAAV as an expression vector. One is that the requirements for delivering a gene to integrate into the host chromosome are surprisingly few. It is necessary to have the 145-bp ITRs, which are only 6% of the AAV genome. This leaves room in the vector to assemble a 4.5-kb DNA insertion. While this carrying capacity may prevent the AAV from delivering large genes, it is amply suited for delivering the antisense constructs of the present invention.

AAV is also a good choice of delivery vehicles due to its safety. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response. AAV therefore, represents an ideal candidate for delivery of the present hammerhead ribozyme constructs.

4.10 Pharmaceutical Compositions and Kits

Pharmaceutical compositions of the present invention will generally comprise an effective amount of at least a first ribozyme, a pair of ribozymes, or a plurality of ribozymes, incorporated into at least a first adeno-associated viral vector, or adeno-associated viral particles containing at least a first ribozyme, a pair of ribozymes, or a plurality of ribozymes, dissolved or dispersed in one or more pharmaceutically acceptable carriers, buffers, solutions, vehicles, or aqueous media.

The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

4.10.1 Parenteral Formulations

The ribozymes, compositions, virus, and AAV-based vectors of the present invention will often be formulated for parenteral administration, e.g., formulated for injection via the intravenous, intramuscular, sub-cutaneous or other such routes. The preparation of an aqueous composition that contains one or more agents, such as a ribozyme, a plurality of ribozymes, a AAV vector, or one or more or adeno-associated virus particles containing one or more such ribozymes, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as freebase or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Compositions comprising the agents of the present invention can be formulated into a composition in a neutral or salt form. Pharmaceutically acceptable salts include the acid addition salts and those formed with inorganic acids such as, for example, hydrochloric or phosphoric acids, or such organic acids as acetic, oxalic, tartaric, mandelic, and the like. Salts can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), dimethylsulfoxide (DMSO), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques, which yield a powder of the active ingredient, plus any additional desired ingredient from a previously sterile-filtered solution thereof. Such injectable solutions may be used, for example, in one or more of the well known surgical methods for directly injecting compounds into the eye, or the subretinal space.

Upon formulation, solutions will be administered in a manner compatible with the dosage formulation and in such amount as is biologically or therapeutically effective. Formulations are easily administered in a variety of dosage forms, such as the type of injectable solutions described above, but drug release capsules and the like can also be employed.

Suitable pharmaceutical compositions in accordance with the invention will generally include an amount of one or more of the agents of the present invention admixed with an acceptable pharmaceutical diluent or excipient, such as a sterile aqueous solution, to give a range of final concentrations, depending on the intended use. The techniques of preparation is generally well known in the art as exemplified by Remington's Pharmaceutical Sciences, 16th Ed. Mack Publishing Company, 1980, incorporated herein by reference. It should be appreciated that endotoxin contamination should be kept minimally at a safe level, for example, less that 0.5 ng/mg protein. Moreover, for human administration, preparations should meet sterility, pyrogenicity, general safety and purity standards as required by FDA Office of Biological Standards.

In addition to the compounds formulated for parenteral administration, such as intravenous or intramuscular injection, other pharmaceutically acceptable forms are also contemplated, e.g., time release or sustained-release formulations, liposomal formulations, microspheres, nanocapsules, and the like. Other pharmaceutical formulations may also be used, dependent on the condition to be treated. Of course, methods for the determination of optimal dosages for conditions such as these would be evident to those of skill in the art in light of the instant specification, and the knowledge of the skilled artisan.

It is contemplated that certain benefits will result from the manipulation of the agents of the present invention to provide them with a longer in vivo half-life. Slow release formulations are generally designed to give a constant drug level over an extended period. Increasing the half-life of a drug, such as agents of the present invention, is intended to result in high intracellular levels upon administration, which levels are maintained for a longer time, but which levels generally decay depending on the pharmacokinetics of the construct.

4.10.2 Therapeutic Kits

The present invention also provides therapeutic kits comprising the agents of the present invention described herein. Such kits will generally contain, in suitable container, a pharmaceutically acceptable formulation of at least a first ribozyme, plurality of ribozymes or adeno-associated virus particles comprising at least a first ribozyme or a plurality of ribozymes, in accordance with the invention. The kits may also contain other pharmaceutically acceptable formulations.

The kits may have a single container that contains the agent, with or without any additional components, or they may have distinct container means for each desired agent. In such kits, the components may be pre-complexed, either in a molar equivalent combination, or with one component in excess of the other; or each of the components of the kit may be maintained separately within distinct containers prior to administration to a patient.

When the components of the kit are provided in one or more liquid solutions, the liquid solution is an aqueous solution, with a sterile aqueous solution being particularly preferred. However, the components of the kit may be provided as dried powder(s). When reagents or components are provided as a dry powder, the powder can be reconstituted by the addition of a suitable solvent. It is envisioned that the solvent may also be provided in another container means. One of the components of the kit may be provided in sealed vials, syringes, or ampules for direct ocular administration.

The container means of the kit will generally include at least one vial, test tube, flask, bottle, syringe or other container means, into which a ribozyme, a plurality of ribozymes, or an AAV vector compositions, or one or more adeno-associated viral particles comprising a ribozyme or plurality of ribozymes, and any other desired agent, may be placed and, preferably, suitably aliquoted. Where additional components are included, the kit will also generally contain a second vial or other container into which these are placed, enabling the administration of separated designed doses. The kits may also comprise a second/third container means for containing a sterile, pharmaceutically acceptable buffer or other diluent.

The kits may also contain a means by which to administer the ribozyme, plurality of ribozymes, AAV-vectors ribozyme, or one or more adeno-associated viral particles comprising one or more of such ribozymes to an animal or patient, e.g., one or more needles or syringes, or even an eye dropper, pipette, or other such like apparatus, from which the formulation may be injected into the animal or applied to a diseased area of the body. The kits of the present invention will also typically include a means for containing the vials, or such like, and other component, in close confinement for commercial sale, such as, e.g., injection or blow-molded plastic containers into which the desired vials and other apparatus are placed and retained.

4.11 Mutagenesis and Preparation of Modified Ribozyme Compositions

Site-specific mutagenesis is a technique useful in the preparation and testing of sequence variants by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a bacteriophage vector that exists in both a single stranded and double stranded form. Typical vectors useful in site-directed mutagenesis include vectors such as the M 13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double stranded vector that includes within its sequence a DNA sequence encoding the desired ribozyme or other nucleic acid construct. An oligonucleotide primer bearing the desired mutated sequence is synthetically prepared. This primer is then annealed with the single-stranded DNA preparation, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected ribozyme using site-directed mutagenesis is provided as a means of producing potentially useful species and is not meant to be limiting, as there are other ways in which sequence variants may be obtained. For example, recombinant vectors encoding the desired gene may be treated with mutagenic agents, such as hydroxylamine, to obtain sequence variants.

4.12 Nucleic Acid Amplification

Nucleic acid, used as a template for amplification, may be isolated from cells contained in the biological sample according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA and is used directly as the template for amplification.

Pairs of primers that selectively hybridize to nucleic acids corresponding to the ribozymes or conserved flanking regions are contacted with the isolated nucleic acid under conditions that permit selective hybridization. The term “primer”, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred.

Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced.

Next, the amplification product is detected. In certain applications, the detection may be performed by visual means. Alternatively, the detection may involve indirect identification of the product via chemiluminescence, radioactive scintigraphy of incorporated radiolabel or fluorescent label or even via a system using electrical or thermal impulse signals (Affymax technology).

A number of template dependent processes are available to amplify the marker sequences present in a given template sample. One of the best-known amplification methods is the polymerase chain reaction (referred to as PCR™), which is described in detail in U.S. Pat. No. 4,683,195, U.S. Pat. No. 4,683,202 and U.S. Pat. No. 4,800,159 (each of which is incorporated herein by reference in its entirety).

Briefly, in PCR™, two primer sequences are prepared that are complementary to regions on opposite complementary strands of the marker sequence. An excess of deoxynucleoside triphosphates is added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in Int. Pat. Appl. Publ. No. WO 90/07641 (specifically incorporated herein by reference). Polymerase chain reaction methodologies are well known in the art.

Another method for amplification is the ligase chain reaction (“LCR”), disclosed in EPA No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qβ Replicase (QβR), described in Int. Pat. Appl. No. PCT/US87/00880, incorporated herein by reference, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]-triphosphates in one strand of a restriction site may also be useful in the amplification of nucleic acids in the present invention.

Strand Displacement Amplification (SDA), described in U.S. Pat. Nos. 5,455,166, 5,648,211, 5,712,124 and 5,744,311, each incorporated herein by reference, is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in Int. Pat. Appl. No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR Gingeras et al., Int. Pat. Appl. Publ. No. WO 88/10315, incorporated herein by reference. In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPA No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., Int. Pat. Appl. Publ. No. WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990, specifically incorporated herein by reference).

Methods based on ligation of two (or more) oligonucleotides in the presence of nucleic acid having the sequence of the resulting “di-oligonucleotide,” thereby amplifying the di-oligonucleotide, may also be used in the amplification step of the present invention.

Following any amplification, it may be desirable to separate the amplification product from the template and the excess primer for the purpose of determining whether specific amplification has occurred. In one embodiment, amplification products are separated by agarose, agarose-acrylamide or polyacrylamide gel electrophoresis using standard methods (see e.g., Sambrook et al., 1989).

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography.

Amplification products must be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled, nucleic acid probe is brought into contact with the amplified marker sequence. The probe preferably is conjugated to a chromophore but may be radiolabeled.

In another embodiment, the probe is conjugated to a binding partner, such as an antibody or biotin, and the other member of the binding pair carries a detectable moiety.

In one embodiment, detection is by Southern blotting and hybridization with a labeled probe. The techniques involved in Southern blotting are well known to those of skill in the art and can be found in many standard books on molecular protocols. See Sambrook et al., 1989. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

### 5. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### 5.1 Example 1

### Construction of Vectors and Expression in Target Cells

5.1.1 rAAV-Ribozyme Constructs

Recombinant AAV constructs were based on the pTR-UF2 vector (Zolotukhin et at., 1996). They resemble the vector used by Flannery et al. (1997) to direct GFP expression to rat photoreceptors except that a 691 bp fragment of the proximal bovine rod opsin promoter replaced the 472 bp murine rod opsin promoter and the ribozyme gene replaced the gfp gene. The bovine promoter fragment contains three proximal promoter elements and the endogenous transcriptional start site at its 3′ end (DesJardin and Hauswirth, 1996) and supports high efficiency, rat photoreceptor-specific expression in vivo. Active and inactive ribozymes were designed, tested and cloned as described above. Each ribozyme gene was followed by an internally cleaving hairpin ribozyme derived from plasmid pHC (Altschuler et al., 1992) resulting in ribozyme cassettes of 140-152 bp. Self cleavage at the internal cutting site in the primary ribozyme RNA leaves identical 3′ ends on each mature ribozyme. The ribozyme cassette was preceded by an intron derived from SV40 and followed by a polyadenylation signal in order to promote nuclear export of the ribozyme. Recombinant AAV titers were determined using both an infectious center assay (Flannery et al., 1997) and a DNAse resistant physical particle assay employing a quantitative, competitive PCR of the neo^{r} gene contained within all rAAV-ribozyme particles (Zolotukhin et al., 1996). Each of the four rAAV-ribozyme virus preparations contained 10¹⁰ to 10¹¹ DNASE resistant particles per ml and 10⁸ to 10⁹ infectious center units per ml. Contaminating helper adenovirus and wild-type AAV, assayed by serial dilution cytopathic effect or infectious center assay respectively, were less than five order of magnitude lower than rAAV.

5.1.2 Subretinal Injection of rAAV

Line 3 albino transgenic rats (P23H-3) on an albino Sprague-Dawley background (produced by Chrysalis DNX Transgenic Sciences, Princeton, N.J.) were injected at the ages of P14 or P15. Animals were anesthetized by ketamine/xylazine injection, and a direction, and b-waves were measured from the cornea-negative peak to the major cornea-positive peak. For quantitative comparison of differences between the two eyes of rats, the values from all the stimulus intensities were averaged for a given animal.

5.1.3 Retinal Tissue Analysis

Rats were euthanized by overdose of carbon dioxide inhalation and immediately perfused intracardially with a mixture of mixed aldehydes (2% formaldehyde and 2.5% glutaraldehyde). Eyes were removed and embedded in epoxy resin, and 1 μm thick histological sections were made along the vertical meridian (26). Tissue sections were aligned so that the ROS and Müller cell processes crossing the inner plexiform layer were continuous throughout the plane of section to assure that the sections were not oblique, and the thickness of the ONL and lengths of RIS and ROS were measured as described by Faktorovich et al. (1990). Briefly, 54 measurements of each layer or structure were made at set points around the entire retinal section. These data were either averaged to provide a single value for the retina, or plotted as a distribution of thickness or length across the retina. The greatest 3 contiguous values for ONL thickness in each retina were also compared to determine if any region of retina (e.g., nearest the injection site) showed proportionally greater rescue; although most of these values were slightly greater than the overall mean of all 54 values, they were no different from control values than the overall mean. Thus, the overall mean was used in the data cited, since it was based on a much larger number of measurements.

5.1.4 RT-PCR™

For quantification of opsin mRNA retina from ribozyme injected or control eyes, retina were isolated without fixation and total RNA immediately extracted using the RNeasy Minikit (Qiagen, Santa Clarita, Calif.). RT-PCR™ was performed using the Pharmacia First-Strand cDNA synthesis kit employing oligo dT as the primer. Wild-type and transgene opsin cDNAs were amplified using a three primer system described above. Primers specific for (3-actin cDNA (Timmers et al., 1993) were included in each reaction for internal standardization.

### 5.2 Example 2

### Endothelial Cell Proliferation in Response to Adenosine Analogues

The subtype of adenosine receptor (A_{2B}) that mediates the proliferative effect of adenosine on HREC was determined by the following studies. The non-selective adenosine receptor agonist NECA, after 48 hr of exposure, induced a concentration-dependent increase of DNA synthesis in HRECs, as indicated by bromodeoxyuridine (BrdU) incorporation. In contrast, neither the A_{2A} adenosine receptor agonist CGS21680 (2-p-(2-carboxyethyl) phenethylamino-5′-N-ethylcarboxamidoadenosine) at concentrations ranging from 10 nM to 10 μM, nor the A₁ adenosine receptor agonist CPA (N⁶-cyclopentyladenosine) at concentrations ranging from 10 nM to 10 μM increased BrdU incorporation by HRECs. The addition of the adenosine receptor antagonist XAC (xanthine amine congener) at 10 μM completely prevented the NECA-stimulated BrdU incorporation. In contrast, neither the selective A₁ adenosine receptor antagonist CPX (8-cyclopentyl-1,3-dipropylxanthine) at 20 nM, nor the selective A_{2A} adenosine receptor antagonist SCH58261 (5-amino-7-(2-phenylethyl)-2-(2-furyl)-pyrazolo(4,3-E)-1,2,4-trizolo(1,5-c) pyrimidine) at 60 nM attenuated the stimulatory effect of NECA on BrdU incorporation by HRECs. These findings indicate that the proliferative effects of NECA are mediated by the A_{2B} adenosine receptor in HREC in culture (Table 3).

TABLE 3
EFFECT OF ADENOSINE RECEPTOR AGONISTS AND ANTAGONISTS ON HREC
(FOLD CHANGE VS. UNTREATED)

| | Proliferation | | | | |
|---|---|---|---|---|---|
| | | BrdU | VEGF | | CAMP |
| | Cell Count | Incorporation | Protein | MRNA | Content |
| Untreated | 1.0 | 1.0 | 1.0 | 1.0 | 1.0 |
| CGS⁽¹⁾ | 1.1 ± 0.03 | 1.0 ± 0.08 | 1.3 ± 0.20 | 0.8 ± 0.37 | 1.1 ± 1.02 |
| CPA (10 μm) | 0.9 ± 0.04 | 0.8 ± 0.06 | 1.1 ± 0.27 | 1.1 ± 0.22 | (ND) |
| NECA (10 μm) | 1.6* ± 0.03 | 1.5* ± 0.05 | 2.7* ± 0.31 | 4.2* ± 0.65 | 11.6* ± 2.36 |
| NECA + CPX | 1.5* ± 0.03 | 1.5* ± 0.05 | 3.2* ± 0.45 | 3.8* ± 1.14 | 9.9* ± 4.01 |
| (20 nM) | | | | | |
| NECA ± SCH⁽²⁾ | 1.6* ± 0.04 | 1.5* ± 0.04 | 3.2* ± 0.23 | 3.6* ± 0.16 | 7.8* ± 2.13 |
| (60 nM) | | | | | |
| NECA + XAC | 1.0 ± 0.03 | 0.9 ± 0.06 | 0.9 ± 0.18 | 0.7 ± 0.26 | 0.8 ± 1.18 |
| (10 μm) | | | | | |
| [NECA + Enprofylline | 1.1 ± 0.04 | (ND) | (ND) | (ND) | (ND) |
| (10 μm) | | | | | |
| NECA + JW | 1.0 ± 0.02 | (ND) | (ND) | (ND) | (ND)] |
| V108 (10 μm) | | | | | |

(ND) = specific condition not tested.
⁽¹⁾Full compound name is CGS21680.
⁽²⁾Full compound name is SCH58261.
*Significantly different from untreated (p < 0.005).

The data for cell counts were consistent with those for BrdU incorporation. Treatment with NECA for 48 hr resulted in a concentration-dependent increase in HREC number, whereas neither CGS21680 nor CPA caused an increase in cell number. Of the three adenosine receptor antagonists tested, only XAC significantly inhibited the increase in cell number induced by 10 μM NECA (Table 3). These compounds, like XAC, blocked the proliferative effects of NECA as measured by cell counts, supporting that the proliferative effects of NECA on HREC are mediated by the adenosine A_{2B} receptor (Table 3).

5.2.1 cAMP Accumulation

Further evidence for the presence of the A_{2B} adenosine receptors in HRECs was obtained by determining the cAMP content of intact HRECs following treatment with adenosine receptor agonists and antagonists (Grant et al., 1999). The cAMP content of intact HRECs following treatment with adenosine receptor agonists and antagonists was next examined. NECA increased cAMP content of HRECs in a concentration-dependent manner, with an EC₅₀ value of 24 μM. In contrast, the selective high affinity A_{2A} adenosine receptor agonist CGS 21680 (at concentrations up to 100 μM) had no significant effect on cAMP content of HREC. The effect of selective A₁ and A_{2A} adenosine receptor antagonists on NECA-induced accumulation of cAMP was also examined. NECA (10 μM)-induced increase in cAMP content in HRECs was not signficantly inhibited by either the selective A_{2A} adenosine receptor antagonist SCH58261 or by the selective A₁ adenosine receptor antagonist CPX. On the other hand, the non-selective adenosine receptor antagonist XAC completely blocked the effect of NECA on cAMP accumulation, support that activation of the adenosine A_{2B} receptor was required for cAMP release in HREC (Table 3). These data indicate that the proliferative effects of NECA were mediated through the A_{2B} receptor subtype.

5.2.2 VEGF Confirmed as the Mediator of Adenosine's Vasoproliferative Effect

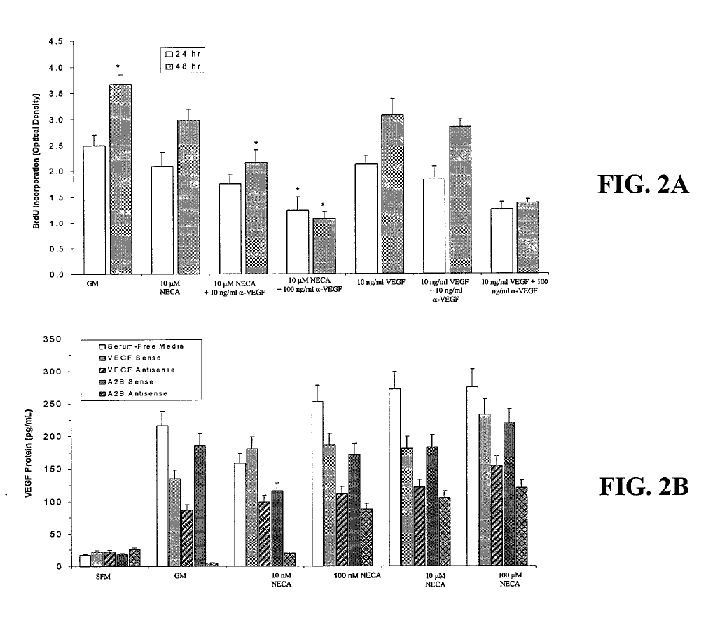

Incubation with VEGF resulted in BrdU incorporation to a level approximating that induced by normal growth medium. The VEGF antibody significantly reduced DNA synthesis induced by VEGF. Incubation with NECA increased DNA synthesis to levels comparable to that induced by normal growth medium. The addition of VEGF antibody resulted in a decrease in NECA-induced BrdU incorporation, which was statistically significant at the highest tested concentration of antibody (FIG. 2A). Similar results were observed at either 24 or 48 hr of exposure to the test agents.

Antisense oligonucleotides for both A_{2B} adenosine receptor and VEGF caused a significant decrease in VEGF in the conditioned medium following NECA exposure (FIG. 2B). This effect was most pronounced for the receptor antisense oligonucleotide with 10 nM NECA, but was evident for all concentrations of NECA tested. The VEGF antisense oligonucleotide also caused a decrease in secreted VEGF in response to NECA, albeit not to the same magnitude as that observed with the A_{2B} adenosine receptor antisense.

NECA induces a dose-dependent increase in ERK activation at 5 and 10 min in HRECs. Both A_{2B} antagonists Enprofylline and JW V-108 abolished ERK activation by NECA. While NECA activated ERK, the A_{2A} agonist, CGS (high doses, 1-10 μM) reduced the basal ERK levels. CGS may be activating cAMP and this cAMP response may down regulate ERK activation. These data support a role for adenosine in the activation of ERK that may then induce the phosphorylation of HIF-1α.

5.2.3 Adenosine A_{2B} Receptor Activation Required for HREC Chemotaxis

The role of the A_{2B} receptor was further characterized by examining the effects of the A_{2B} antagonists JW V108 and Enprofylline on HREC chemotaxis, capillary tube formation and signal transduction pathways following stimulation with the adenosine analogue NECA. NECA induced HREC chemotaxis in a concentration-dependent manner that was inhibited by Enprofylline and JW V108.

5.2.4 Adenosine A_{2B} Receptor Required for HREK ERK Activation

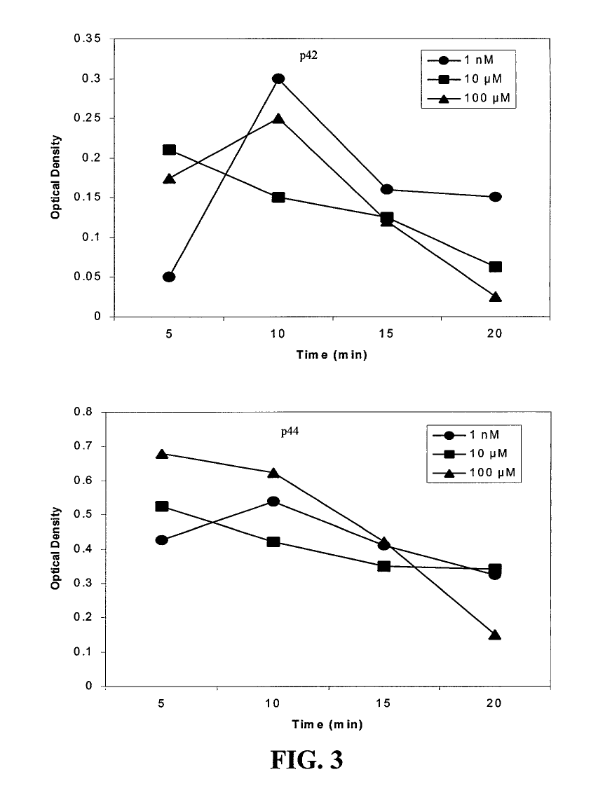

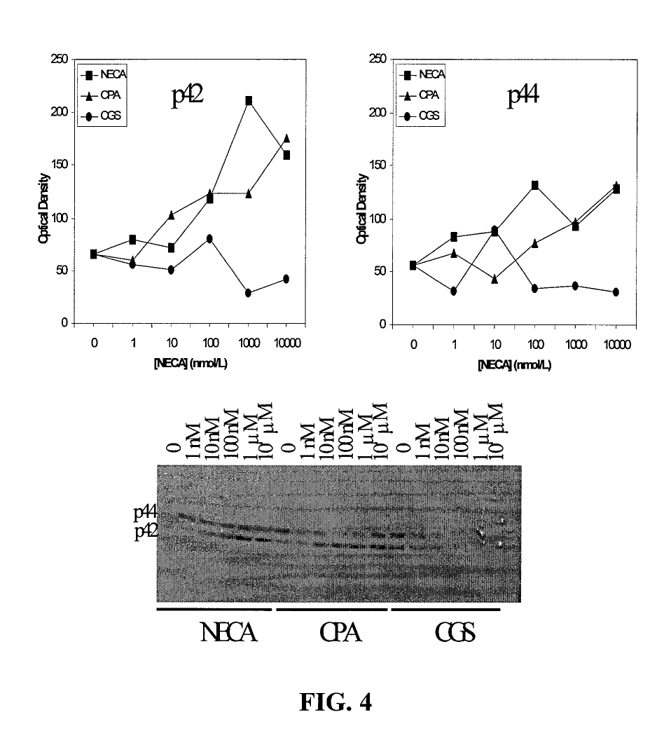

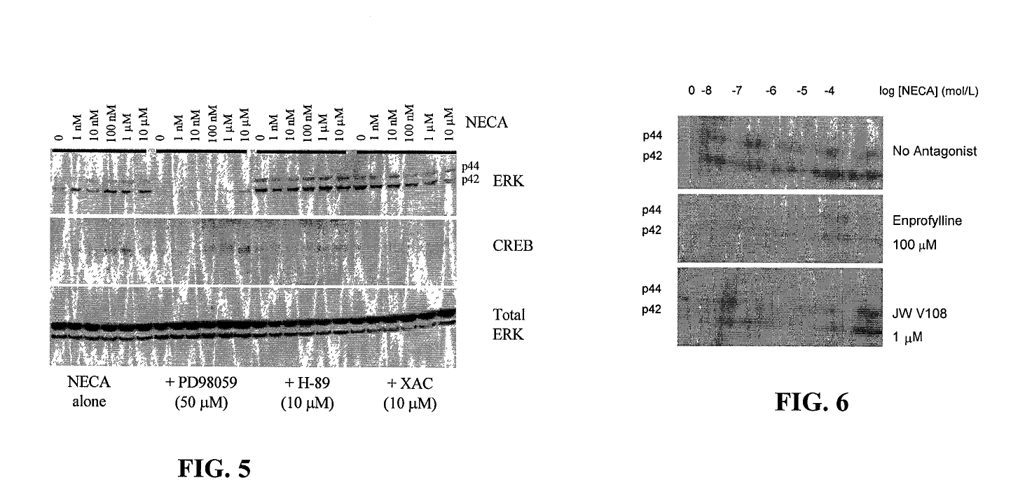

NECA (1 nmol/L-10 μmol/L) induced a transient activation of ERK which peaked at 5 min and desensitized within 20 min. The rate of desensitization was dependent on NECA concentration since higher doses of NECA produced a more rapid desensitization (FIG. 3). The A₁-selective agonist CPA was also capable of stimulating ERK (FIG. 4), however the A_{2A}-selective agonist CGS did not activate ERK. In order to determine the intracellular signaling pathways activated by NECA that regulate ERK activity, we pretreated cells for 30 min with the ERK/MPAK kinase (MEK) inhibitor PD98059 or the PKA inhibitor H-89 and stimulated with NECA for 5 min. PD98059 abolished ERK activation, while H-89 increased basal ERK activation (FIG. 5). H-89 did not block NECA-stimulated ERK activation, suggesting that PKA is not involved in signaling from the adenosine receptor to ERK. The non-selective adenosine receptor antagonist XAC decreased ERK activation by high concentrations of NECA, but modestly increased ERK activation in control conditions and in response to 1 and 10 nM NECA. Interestingly, prolonged activation with NECA in the presence of XAC or SCH and CPX reduced the rate of ERK desensitization, suggesting that adenosine receptors are involved in both activation and desensitization of ERK.

Phosphorylation of cAMP response element binding protein (CREB) at Ser¹³³ was examined following NECA stimulation in order to determine whether activation of cAMP pathways by NECA occurred independently of ERK activation. Cells were pretreated with PD98059 or H-89 and assayed for active CREB by western blot. PD98059 did not alter CREB activation, however both H-89 and XAC blocked CREB phosphorylation. These data indicate that ERK activation by NECA occurs independently of the cAMP response (FIG. 5).

Enprofylline and JW V108 exhibit greater selectivity for the A_{2B} receptor. Cells were pre-treated with both antagonists for 10 min and stimulated with increasing concentrations of NECA. Enprofylline completely abolished ERK activation, while JW V108 inhibited ERK activation at all concentrations except for 10 μM. These data suggest that ERK activation occurs through both the A_{2B} and A₁ receptors, but not the A_{2A} receptor (FIG. 6). These data support a role for adenosine in the activation of ERK that may then induce the phosphorylation of HIF 1-α.

### 5.3 EXAMPLE 3

### Development and Testing of Ribozyme Targeting A_{2B} Adenosine Receptor mRNA

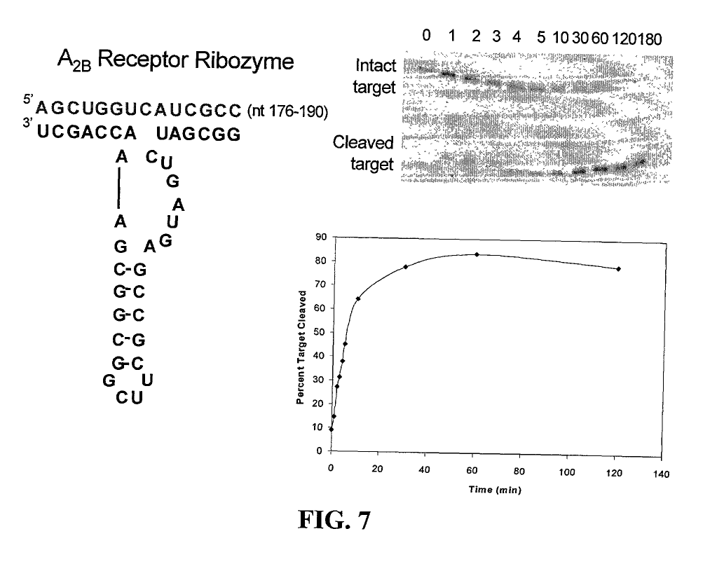

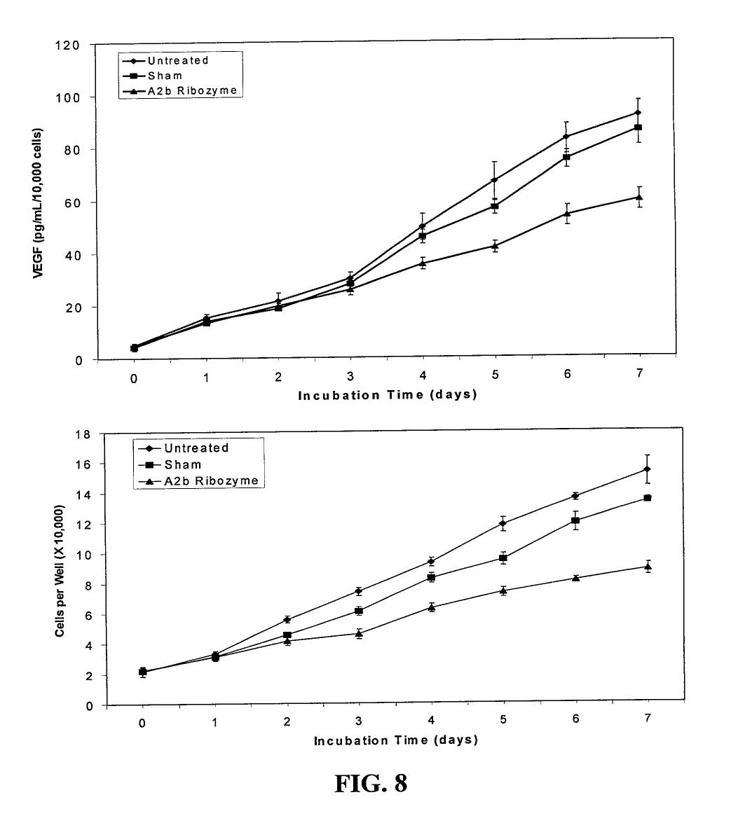

The cleavage site of the A_{2B} antisense, between nucleotides 183 and 184, was demonstrated to be accessible within the secondary structure of the native mrRNA by the antisense studies. A hammerhead ribozyme designed to cleave this message was then synthesized along with a 14-nucleotide target sequence (FIG. 7). This target was end-labeled in a standard kinase reaction with ³²P, then incubated along with ribozyme (1:1 molar ratio) for 1, 2, 3, 4, 5, 10, 30, 60, 120 and 180 min. Nearly 90% of target was cleaved by 60 min (FIG. 7), demonstrating the efficacy and rapid action of this ribozyme in a cell-free assay system. The ribozyme's effects on HREC proliferation and VEGF synthesis in response to adenosine receptor activation was examined. HRECs were plated in serum-free medium overnight to adhere and make them quiescent. Unattached cells were then removed by washing with Hank's balanced salt solution (HBSS). The cells were then incubated with 1 U/mL adenosine deaminase (ADA) for 20 min, after which was added either medium alone, 1 μmol/L A_{2B} receptor ribozyme, or 1 μmol/L of a synthetic mixed oligonucleotide of the same length as the ribozyme, all of which contained 10 μmol/L NECA. Cells were then incubated for a total of seven days. Sampling occurred every 24 hr as follows. Conditioned medium was collected and stored at −70° C. until the end of the assay, after which it was analyzed for VEGF using a commercially available ELISA. The cells were enzymatically dissociated from the wells and counted using a Coulter counter. These latter results were then used to normalize the VEGF data to a constant cell number. FIG. 8 shows that cells treated with ribozyme express up to 60% less VEGF protein in response to NECA than do either untreated cells or cells treated with sham oligonucleotide. Similarly, these same cells exhibited a 50% reduction in proliferation 7 days after NECA stimulation when exposed to ribozyme compared to control.

5.3.1 Oxygen-Induced Retinal Neovascularization in the Neonatal Mouse

The potential efficacy of administering adenosine receptor antagonists to reduce retinal neovascularization brought on by ischemic insult was examined. In the neonatal mouse model of oxygen-induced retinopathy, 7-day-old mice are placed with their nursing dams in a 75% oxygen atmosphere for 5 days. Upon return to normal air, these mice develop retinal neovascularization, with peak development occurring 5 days after their return to normoxia. During this time, the animals receive by daily intaperitoneal injection a pharmacologically relevant concentration of adenosine receptor antagonist or vehicle (0.15% vol./vol. DMSO in normal saline) alone. Exemplary antagonists tested include XAC and 3-n-propylxanthine (Enprofylline), and JW V108, both at a concentration of 30 mg/Kg of body weight.

At the fifth day after return to normoxia, the animals were sacrificed and the eyes removed for fixation in sodium cacodylate buffered acrolein (5% vol./vol.). After extensive washing, the eyes were embedded in epoxy resin for sectioning. At least twenty sections, 1 μm thick with 5 μm between sections, were cut sagitally from each eye, resulting in a total sampling thickness of 120 μm for each eye. Sections were then stained to visualize cell nuclei.

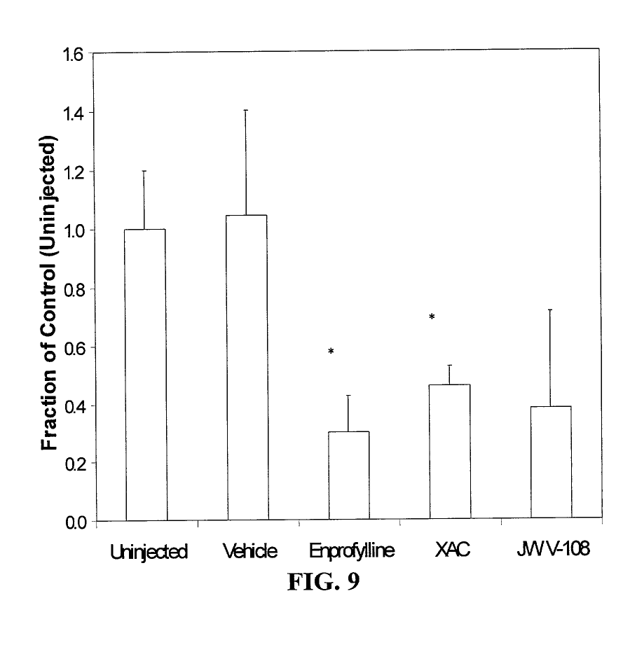

Individuals masked to the identity of treatment counted all cell nuclei above the inner limiting membrane for all 20 sections from each eye. These data were then expressed as the sum of the counts from each eye. The efficacy of treatment with a particular antagonist was then calculated as the fraction of total nuclei in antagonist or vehicle treatment over total nuclei in uninjected control. FIG. 9 summarizes these findings and shows that 3 adenosine receptor antagonists tested inhibit oxygen-induced retinal neovascularization by 54% for XAC and 70% for Enprofylline. Selected animals were anesthetized and then perfused with 10 mL 50 mg/ml fluorescein-Dextran 2,000,000 in formaldehyde (4%) via cardiac puncture. Whole retinas from these eyes were then flat-mounted for qualitative assessment of retinal neovascularization by fluorescence microscopy.

5.3.2 Laser-Induced Venous Thrombosis in Mouse Eyes

Pilot studies were performed to determine the feasibility of inducing retinal neovascularization in the mouse eye by occluding each branch vein via laser photocoagulation as described. There occurs preretinal neovascularization as a result of profound retinal ischemia. The presence of corneal and iris neovascularization also supports the presence of severe retinal ischemia with the release of growth factors into the vitreous that ultimately reach the cornea and cause neovascularization.

The ability to differentiate the source of endothelial cells responsible for retinal vasculogenesis following photodynamic venous thrombosis can be accomplished using the NOD.B10^{B6.gfp} chimeric mouse. Re-infiltration of vascular areas by bone marrow-derived, nucleus-containing cells from reconstituted bone marrow in the NOD.B10^{B6.gfp} mouse results in easy detection of bone marrow-derived cells by virtue of their expressing gfp. This demonstrates the feasibility of examining retinal vasculature for gfp expression as an indicator of newly formed vessels following photodynamic venous coagulation and/or treatment with adenosine analogues to stimulate endothelial cell migration and proliferation.

### 5.4 Example 4

### Blood Analysis for CD34⁺Progenitor Cells in Diabetic Patients with PDR

CD34⁺ progenitor cells were isolated from the leukocyte fraction of blood from two patients who were experiencing rapid deterioration of their vision associated with new onset retinal neovascularization. These samples were analyzed by flow cytometry. The number of CD34⁺ cells detected in the serum of one patient was 30-fold higher than those detected in a non-diabetic control patient sample. The second patient was 15-fold higher than non-diabetic control levels. This intriguing finding is being pursued as part of a multicenter study involving patients with severe NPDR and will not be pursued here. This observation does, however, establish the justification for examining the correlation between circulating angioblasts and IGF-I/VEGF serum levels.

5.4.1 Production of Bone Marrow Chimeric Mice

C57BL/6-gfp transgenic mice were maintained through selected brother-sister matings. Because homozygosity at the gfp-transgene is lethal at day E14 of fetal development, breeding pairs are established consisting of C57BL/6-gfp^{-/-} males and C57BL/6-gfp^{+/-} females. The offspring from such breeding pairs are approximately 50% non-fluorescent and 50% fluorescent within both sexes. This represents an ideal situation for the production of bone marrow chimeras since gfp⁺ bone marrow can be introduced into syngeneic gfp⁻ (non-fluorescent) siblings. Production of C57BL/6-gfp bone marrow chimeras were carried out as described previously by the Co-PI (LaFace and Peck, 1989).

5.4.2 HIF-1α Levels in Response to Adenosine

Mouse monoclonal antibodies (IgG) which recognize the C-terminus of HIF-1α were obtained from Novus Biologicals. This reagent is used to monitor stability and phosphorylation of HIF-1α following treatment of HREC with the non-specific adenosine receptor agonist NECA at concentrations of 10 nM-10 μM (Richard et al., 1999). HREC is exposed to varying O₂ concentrations (1, 3, 10%) to induce hypoxia and will be used for comparison to NECA treated cells. After treatment, cells will be lysed in a buffer containing Triton X-100, 100 mM NaCl and a combination of proteinase and phosphatase inhibitors as previously described (Davis et al., 1999). Protein concentration is determined by the BCA assay and 5-10 μg of nuclear extract was separated by electrophoresis on 8% SDS polyacrylamide gels. Proteins are electrophoretically blotted to PDVF and incubated with a 1:1000 dilution of antibodies to HIF-1α. The presence of immunoreactivity is detected by enhanced chemiluminscence (ECL, Amersham) for qualitative experiments. To confirm that slowly migrating forms correspond to phosphorylated HIF-1α, extracts are treated with phosphatase (lambda phosphatase, NEB) in the absence of phosphatase inhibitors prior to electrophoresis (Forsythe et al., 1996). In the next series of experiments, 5-10 μg of nuclear extract is incubated with a ³²P-labeled 47 bp double-stranded DNA probe based on the HRE of the VEGF gene. Competition experiments include excess unlabeled oligonucleotide. In this assay, a dose-dependent increase in the amount of shifted VEGF probe following treatment of cells with NECA is expected. This shift is competed by unlabeled hypoxia response element (HRE) DNA based on the VEGF or erythropoietin promoter.

5.4.3 Involvement of Hypoxia Response Element in Response to Adenosine

The adenosine-responsive region of the VEGF promoter is analyzed in a manner similar to Forsythe et al. who mapped the hypoxia response element (HRE) of the VEGF gene upstream of the transcription initiation site (Forsythe et al, 1996). A 1.6 kb fragment of VEGF genomic DNA that contains 1.2 kb of 5′ flanking sequence and 0.4 kb of primary transcript was ligated into the luciferase-reporter plasmid pGL2 (Promega). This plasmid contains an intron and polyadenylation signal from SV40 but lacks proximal promoter and enhancer elements. The promoter dissection by Forsythe et al. (1996) as well as others (Shima et al., 1996) relied on convenient restriction sites to generate promoter deletions, but an inverse PCR™ technique (Hemsley et al., 1989) may be used to generate a set of internal deletions of the VEGF promoter. Because primers can be placed with precision, PCR™-generated deletion permits better discrimination of closely spaced promoter elements than methods that depend on timed exonuclease reactions (Xu and Gong, 1999). Selected regions of the VEGF promoter were deleted using divergent primers and a commercial PCR™ mix designed to promote long-range PCR™. The PCR™ product was then circularized by ligation. To avoid PCR™-generated mutations elsewhere in the plasmid (especially in the luciferase gene), a second set of primers was added to amplify the deleted promoter region, which was then re-cloned in the reporter plasmid. The upstream regions were sequenced to exclude unintentional PCR™ mutagenesis.

Human retinal endothelial cells are maintained in culture as previously described (Grant et al., 1999). Based on the DEAE-Dextran method (Agarwal et al., 1998; Selden, 1993), HRECs may be grown to about 50% confluence prior to transfection with 1.5 ml DEAE-Dextran solution/100 mm plate containing 5.0 μg DNA of the vectors. The cells are washed twice with Tris-buffered saline and media containing 10% NuSerum is added to the plates. The transfection solution is then added to each dish drop by drop equally over each portion of the plate and then gently swirled. To increase transfection efficiency, chloroquine diphosphate (100 μM) is added to the medium at this stage, and cells are incubated for 4 hr at 37° C. in 5% CO₂/room air. The cells are shocked for 1 min at room temperature by the addition of 10% DMSO in PBS, washed with PBS, then chloroquine free medium is added to each plate. In addition to the VEGF-luciferase plasmid, cells are co-transfected with pSVbgal, to serve as a measure of transfection efficiency. Cells from duplicate transfections are allowed to recover for 24 hr in 6-well plates (Costar). Cells are then given fresh medium containing 10 nM-10 μM NECA or vehicle, and incubated a further 30 min to 4 hr in 5% CO₂, 95% air at 37° C. during which time luciferase activity is measured using a fluorescence microplate reader. β-galactosidase activity is measured by hydrolysis of 2-nitrophenyl-β-D-galactopyranoside. Relative luciferase activity is calculated as light units produced normalized to β-galactosidase activity and protein concentration on cell lysates as measured by BCA assay (Pierce).

5.4.4 Pharmacological Analysis of Adenosine-Stimulated VEGF Expression

Previously it was demonstrated that NECA stimulates VEGF expression through the A_{2B} receptor (Grant et al., 1998). These experiments relied on selectively blocking A₁ and A_{2A} receptors and determining the A_{2B} component by subtracting the A₁ and A_{2A} responses. Since this study was published, selective A_{2B} antagonists have been obtained that also block NECA-stimulated ERK activation. These experiments support the role of the A_{2B} receptor in NECA-stimulated proliferation, however the intracellular signal transduction pathways from the receptor to the nucleus and VEGF promoter have not been defined. The A_{2B} receptor has been shown to signal through two separate G-protein-coupled signal transduction pathways in several cell types (D'Angelo et al., 1997; Wu et al., 1993; Cook and McCormick, 1993). The “classical” A_{2B} pathway activates adenylyl cyclase through Gs, while recent evidence suggests that the A_{2B} receptor also couples through Gq/11 to activate phospholipases and PKC (D'Angelo et al., 1997; Wu et al., 1993; Cook and McCormick, 1993). In order to define transcription factors that regulate VEGF expression, it is necessary to define the NECA-stimulated signal transduction pathways.

HRECs transfected with the luciferase/HRE constructs (described above) are assayed in a 96-well plate format using a temperature-controlled microplate luminometer as described above. Cells are assayed at 1 hr intervals and are assayed with activators and inhibitors under hypoxic and normoxic conditions. Negative controls contain DMSO or PBS at the concentration used as a vehicle. Plates are sealed in a chamber under the appropriate oxygen concentration and 10 mM HEPES is added to the assay medium to maintain pH. In experiments using NECA as an activator, the assay is performed as a dose-response (10 nM to 10 μM) in the presence of inhibitors and in the presence and absence of the A_{2B} receptor antagonists, Enprofylline and JW V108. Inhibitors are used at 10 times the inhibition constant. In experiments where activators are being used (e.g., forskolin, 8-Br-cAMP, phorbol esters), luciferase activity is assayed as a dose response to the activator. The role of AC and PKA in VEGF regulation is determined by pre-treating cells with cholera toxin, forskolin, H-89 and 8-Br-cAMP. If A_{2B}-stimulation of the AC/cAMP/PKA pathway is responsible for NECA-stimulated VEGF expression, then cholera toxin and 8-Br-cAMP and forskolin should increase VEGF promoter activity in the absence of NECA, while H-89 and 8-Br-Rp-cAMP should block NECA-stimulated luciferase activity if transcription is mediated by cAMP-dependent pathways. However, it is possible that A_{2B} coupling through members of the Gq/11 family is responsible for NECA-stimulated VEGF transcription. The hypothesis may be tested by stimulating the cells with NECA and selectively inhibiting this pathway at multiple points. The Gq-selective antagonist GP Antagonist 2A (Mukai et al., 1992) will be used to block signaling at the level of receptor coupling. Because Gq can activate two initially divergent pathways, both intracellular Ca⁺⁺ chelators BAPTA/AM and PKC inhibitors (Go 6976 for Ca⁺⁺ dependent PKC isoforms, Calphostin C as a general inhibitor) are tested for their ability to block VEGF induction stimulated by NECA. Similarly, the PKC activator PMA (phorbol 12-myristate-13-acetate) and thapsagargin (which increases intracellular Ca⁺ by inhibiting the endoplasmic reticulum Ca⁺⁺-ATPase) are tested for their ability to substitute for NECA in stimulating luciferase activity. If VEGF induction is due to A_{2B} activation of PKC or increased intracellular calcium, then PMA and thapsagargin, respectively, will substitute for NECA. Finally, the ERK (MEK) inhibitor PD98059 will be added to experiments showing increased VEGF expression in order to determine the role of ERK in VEGF induction.

5.4.5 Western Analysis of HIF-1α

Cells are lysed in a buffer containing Triton X-100, 100 mM NaCl and a combination of proteinase and phosphatase inhibitors as previously described (Davis et al., 1999). Protein concentration is determined using the BCA assay. Five-ten μg of nuclear extract is separated by electrophoresis on 8% SDS polyacrylamide gels. In the first series of experiments, proteins are electrophoretically blotted to PDVF and incubated with a 1:1000 dilution of antibodies to HIF1α. The presence of immunoreactivity is detected by enhanced chemiluminscence (ECL, Amersham) for qualitative experiments.

5.4.6 Preparation of Luciferase-Reporter Plasmids

A 363 bp fragment of the VEGF promoter has been cloned into the pGL2-Basic plasmid (Promega). The VEGF promoter was inserted upstream of firefly luciferase and drives the expression of this protein. This 336 bp fragment contains a 33 bp region encoding the hypoxia response element (HRE). This HRE may be deleted using inverse PCR™ as described (Hemsley et al., 1989) and this construct and the wild type version may be used to test the effect of NECA on the activation of the VEGF promoter.

5.4.7 Transfection of HRECs

Based on the DEAE-Dextran method (Agarwal et al., 1998; Selden, 1993), HRECs are grown to about 50% confluence prior to transfection with 1.5 ml DEAE-Dextran solution/100 mm plate containing 5.0 μg DNA of the vectors. The cells are washed twice with Tris-buffered saline and media containing 10% NuSerum is added to the plates. The transfection solution is then added to each dish drop by drop equally over each portion of the plate and then gently swirled. To increase transfection efficiency, chloroquine diphosphate (100 μM) is added to the medium at this stage. Cells are incubated for 4 hr at 37° C. in 5% CO₂/room air. The cells are shocked for 1 min at room temperature by the addition of 10% DMSO in PBS, washed with PBS, then chloroquine free medium is added to each plate.

5.4.8 Dual-Lucifierase Reporter Assay

Transfected cells are given fresh medium containing 10 nM-10 μM NECA or vehicle, and incubated a further 30 min to 4 hr in 5% CO₂, 95% air at 37° C. during which time luciferase activity is measured using a fluorescence microplate reader. β-galactosidase activity is measured by hydrolysis of 2-nitrophenyl-β-D-galactopyranoside. Relative luciferase activity is calculated as light units produced normalized to β-galactosidase activity and protein concentration on cell lysates as measured by BCA assay (Pierce).

5.4.9 Promoter Studies

Because primers can be placed with precision, PCR™-generated deletion permits better discrimination of closely spaced promoter elements than methods that depend on timed exonuclease reactions. The method of Xu and Gong (199) may be used. Selected regions of the VEGF promoter are deleted using divergent primers and a commercial PCR™ mix designed to promote long-range PCR™. The PCR™ product is then circularized by ligation. To avoid PCR™-generated mutations elsewhere in the plasmid (especially in the luciferase gene) a second set of primers is added to amplify the deleted promoter region, which is then re-cloned in the reporter plasmid. The upstream regions are sequenced to exclude unintentional PCR™-mutagenesis.

### 5.5 Example 5

### Design and Testing of A_{2B} Receptor-Specific Ribozymes

A hammerhead ribozyme was designed to cleave the mRNA for the A_{2B} receptor following nucleotide 183 (FIG. 6). This site was demonstrated to be accessible within the folded structure of the mRNA based on experiments using antisense oligodeoxynucleotides to inhibit expression of A_{2B} in tissue culture (Grant et al., 1999).

Because the viral rep gene is missing from AAV vectors, site-specific integration does not occur, but the vector appears to randomly integrate into host DNA randomly (Kearns et al., 1996; Ponnazhagan et a., 1997). Recombinant AAV (rAAV) vectors have been used successfully in long-term gene delivery to the retina (Flannery et al., 1997; Bennett et al., 1997), the lung (Flotte et al., 1993), muscle (Kessler et al., 1996; Xiao and Samulski, 1996), brain (Klein et al., 1998; Xiao et al., 1997), spinal cord (Peel et al., 1997), liver (Snyder et al., 1997) and blood vessels (Rolling et al., 1997). In particular, AAV infects vascular endothelial cells in vivo (Gnatenko et al., 1997; Lynch et al., 1997). A single intravitreal injection of AAV expressing marker proteins (β-galactosidase or gfp) was able to genetically transduce a variety of cell types in the guinea pig eye, including blood vessels, for up to one year post injection with no evidence of inflammation or other abnormalities (Guy et al., 1999). Unlike adenovirus vectors used in gene therapy trials, AAV does not cause inflammation and does not provoke a cell-mediated immune response (Bennett et al., 1997; Bennett et al., 1999). Unlike retroviral vectors, AAV is able to infect non-cycling cells, such as vascular endothelial cells. Retroviral vectors have been used for dividing endothelial cells in culture, but not for non-dividing cells in vivo. The PGK1 HRE promoter may be used. As an alternative to the PGK1 promoter, the adenosine-responsive region of VEGF in conjunction with the SV-40 proximal promoter elements may be used in order to regulate expression of adenosine receptor ribozymes. In this way, an autologously regulating feedback-loop may be established to reduce the expression of the A_{2B} receptor.

5.5.1 Animals

Male NOD.Gfp^{-/-} mice are gamma-irradiated (650-850 R) and placed on acid water (pH 2.0). Between 4-6 hrs after irradiation, the recipient mice are reconstituted with bone marrow from male NOD.Gfp^{-/+} mice (whole bone marrow isolated from the long bones of the front and hind legs of C57BL/6-gfp^{-/+} mice) injected intravenously via the tail vein. Reconstitutions are carried out using 10⁶-10⁷ bone marrow cells per recipient. After reconstitution, the mice are observed daily for signs of wasting disease or other complications; however, survival approaches 100%. Successful reconstitution is determined by flow cytometric analysis of a drop of blood. Leukocytes constitutively produce VP (thus there is no dilution of fluorescence in daughter cells) while erythrocytes remain non-fluorescent.

The NOD.Gfp mouse line was derived from breeding female NOD mice with a C57BL/6-gfp transgenic male mouse. Gfp-positive offspring were backcrossed to NOD mice at each generation. Animals are followed closely for the onset of diabetes. Diabetic animals may receive one or more units of humulin NPH in the evening. Their blood sugars are measured once every two weeks or, if necessary, more frequently.

5.5.2 Quantifying Circulating Angioblasts

Mice are anesthetized deeply with ketamine/xylazine (70 mg/kg, 15 mg/kg, respectively) and exsanguinated by cardiac puncture. Low-density mononuclear cells (less than 1.077 g/ml) are recovered by density centrifugation using Ficoll-Hypaque to enrich their numbers for subsequent analysis. Circulating angioblasts are enumerated using PE-conjugated anti-CD34 antibody staining. This allows differentiation of angioblasts from the total leukocyte population as well as quantification by two-channel flow cytometry. All leukocytes exhibit green fluorescence (via g expression), but only angioblasts exhibit concomitant red fluorescence (via specific Ab binding).

5.5.3 Preparation of Retinal Whole Mounts

Mice are anesthetized as described above, then perfused through the left ventricle with 4% paraformaldehyde in 0.1 M phosphate buffer pH 7.4. The eyes are enucleated and placed in 4% paraformaldehyde for 24 hr. The retinas are dissected from the globes and incised with four radial cuts to allow flat mounting with glycerol-gelatin. The flat mounted retinas are viewed by fluorescence microscopy and photographed. One thousand capillary cells are counted to determine the percentage which exhibit gfp expression.

5.5.4 The C57BL/6./gfp Transgenic Mouse and Production of NOD.B10^{B6.gfp} Chimeric Mice

C57BL/6-gfp⁺ transgenic mice are maintained through selected brother-sister matings. Because homozygosity at the gfp-transgene is lethal, breeding pairs are established consisting of C57BL/6-gfp^{-/-} males with C57BL/6-gfp^{+/-} females (where C57BL/6-gfp^{+/-} represents “near” homozygous mice). The offspring, then, are approximately 50% non-fluorescent and 50% fluorescent. This represents an ideal situation for the production of bone marrow chimeric mice since the gfp⁺ bone marrow can be introduced into syngeneic gfp⁻ (non-fluorescent) siblings. Production of bone marrow chimeric mice may be carried out as detailed previously by LaFace and coworkers (1989). In brief, at time of hematopoietic stem cell reconstitution, young adult NOD.B10 mice (5-6 weeks of age) are gamma-irradiated (650-850 R) and placed on acid water (pH 2.0). Between 4-6 hrs after irradiation, the reconstituting cell population (whole bone marrow isolated from the long bones of the front and hind legs of C57BL/6-gfp⁺) are injected into each host intravenously via the tail vein. Reconstitution is carried out using 10⁶-10⁷ bone marrow cells per recipient. After reconstitution, the mice are observed daily for signs of wasting disease or other complications, however, successful reconstitution typically approaches 100%.

5.5.5 Identification of Differentially Expressed Genes During Ischemia Induced Angiogenesis

On days 2, 4, 7, 10 and 14 following laser treatment, mice are euthanized and their eyes removed. Retinas are removed and collected for analysis to determine temporal changes in gene expression that may lead to the recruitment of angioblasts and the development of preretinal neovascularization. Total RNA is isolated from each retina for use in microarrays and compared to the RNA obtained from the untreated eyes. If angioblasts are bone marrow-derived, as indicated by expression of gfp, then gfp-positive cells are collected from the preretinal tufts by laser capture using the Bio-Rad 1024ES Confocal Microscope. Total RNA is extracted from these captured cells and compared to RNA from neighboring retinal endothelial cells (resident cells) as well as from angioblasts obtained from peripheral blood as described above (Takahashi et al., 1999). Gene expression for all of these RNA isolates is determined by cDNA microarray analysis.

TABLE 4
ILLUSTRATIVE HAIRPIN RIBOZYME TARGETS OF THE PRESENT INVENTION

SEQUENCE
Cleavage site

| RIBOZYME | HelixII | | | |
|---|---|---|---|---|
| ROD OPSIN MRNA-SPECIFIC: | ↓ | Helix I | SEQ ID NO: | REFERENCE |
| P23L target: | acgc a gcc | ucuucg-3′ | SEQ ID NO:3 | Berson et al., 1991 |
| Ribozyme arms: | ugcg aaga | agaagc-5′ | | |
| F45L target: | acau g guu | cugcug | SEQ ID NO:4 | Sung et al., 1991 |
| Ribozyme arms: | ugug aaga | gacgac | | |
| G51A target: | ugcu g gcc | uucccc | SEQ ID NO:5 | Macke et al., 1993 |
| Ribozyme arms: | acgg aaga | aagggg | | |
| G51G target: | ugcu g guc | uucccc | SEQ ID NO:6 | Dryja et al., 1991 |
| Ribozyme arms: | acgg aaga | aagggg | | |
| P53R target: | gcug g gcu | uccggc | SEQ ID NO:7 | Inglehearn et al., 1992 |
| Ribozyme arms: | cgac aaga | aggccg | | |
| QG4stop target: | ucac c guc | uagcac | SEQ ID NO:8 | Macke et al., 1993 |
| Ribozyme arms: | agug aaga | aucgug | | |
| G90D target: | aggu g gcu | ucacca | SEQ ID NO:9 | Sieving et al., 1992 |
| Ribozyme arms: | uccg aaga | aguggu | | |
| G106W target | uucu g gcc | ccacag | SEQ ID NO:10 | Sung et al., 1991 |
| Ribozyme arms: | aagg aaga | gguguc | | |
| G114D target: | ugga g gac | uucuuu | SEQ ID NO:11 | Vaithinathan et al., 1994 |
| Ribozyme arms: | accu aaga | aagaaa | | |
| R135L target: | aucg a guu | guacgu | SEQ ID NO:12 | Jacobson et al., 1991 |
| Ribozyme arms: | uagc aaga | caugca | | |
| R135P target: | aucg a gcc | guacgu | SEQ ID NO:13 | Rodriguez et al., 1993 |
| Ribozyme arms: | uagc aaga | caugca | | |
| P180A target: | acau c gcc | gagggc | SEQ ID NO:14 | Daiger et al., 1995 |
| Ribozyme arms: | ugug aaga | cucccg | | |
| D190G target: | aauc g gcu | acuaca | SEQ ID NO:15 | Dryja et al., 1991 |
| Ribozyme arms: | uuag aaga | ugaugu | | |
| H211R target: | ucgu g guc | cgcuuc | SEQ ID NO:16 | Macke et al., 1993 |
| Ribozyme arms: | agcg aaga | gcgaag | | |
| H211P target: | ucgu g guc | cccuuc | SEQ ID NO:17 | Macke et al., 1993 |
| Ribozyme arms: | agcg aaga | gggaag | | |
| F220C target: | cauc u guu | ucugcu | SEQ ID NO:18 | Bunge et al., 1993 |
| Ribozyme arms: | guag aaga | agacga | | |
| P347S target: | aggu g gcc | ucggcc | SEQ ID NO:19 | Dryja et al., 1990 |
| Ribozyme arms: | uccg aaga | agccgg | | |

TABLE 5

ILLUSTRATIVE HAMMERHEAD RIBOZYME TARGETS OF THE PRESENT INVENTION

| | SEQUENCE | SEQ ID NO: | |
|---|---|---|---|
| RIBOZYME | Target reads 5′ to 3′ | ribozyme reads 3′ to 5′ | REFERENCE |
| ROD OPSIN MRNA-SPECIFIC: | | | |
| P23H target: | gccacuu cgagua | SEQ ID NO:20 | Berson et al., 1991 |
| ribozyme arms: | cgguga gcucau | | |
| P23L target: | gccucuu cgagua | SEQ ID NO:21 | Dryja et al., 1991 |
| ribozyme arms: | cggaga gcucau | | |
| Q28H target: | cacacua cuaccu | SEQ ID NO:22 | Bunge et al., 1993 |
| ribozyme arms: | guguga gaggga | | |
| F45L target: | augguuc ugcuga | SEQ ID NO:23 | Sung et al., 1991 |
| ribozyme arms: | uaccaa acgacu | | |
| L46R target: | auguuuc ggcuga | SEQ ID NO:24 | Rodriguez et al., 1993 |
| ribozyme arms: | uacaaa ccgacu | | |
| G51R target: | ugcgcuu ccccau | SEQ ID NO:25 | Dryja et al., 1992 |
| ribozyme arms: | acgcga ggggua | | |
| G51A target: | uggccuu ccccau | SEQ ID NO:26 | Macke et al., 1993 |
| ribozyme arms: | accgga ggggua | | |
| G51V target: | uggucuu ccccau | SEQ ID NO:27 | Dryja et al., 1991 |
| ribozyme arms: | accaga ggggua | | |
| P53R target: | ugggcuu ccgcau | SEQ ID NO:28 | Inglehearn et al., 1992 |
| ribozyme arms: | acccga ggcgua | | |
| T58R target: | cuuccuc aggcuc | SEQ ID NO:29 | Bunge et al., 1993 |
| ribozyme arms: | gaagga uccgag | | |
| T58R target: | caggcuc uacguc | SEQ ID NO:30 | Bunge et al., 1993 |
| ribozyme arms: | guccga augcag | | |
| Q64stop target: | caccguc uagcac | SEQ ID NO:31 | Macke et al., 1993 |
| ribozyme arms: | guggca aucgug | | |
| Q64stop target: | ccgucua gcacaa | SEQ ID NO:32 | Macke et al., 1993 |
| ribozyme arms: | ggcaga cguguu | | |
| Δ68-71 target: | ugaacua cauccu | SEQ ID NO:33 | Keen et al., 1991 |
| ribozyme arms: | acuuga guagga | | |
| V87D target: | ggaccua gguggc | SEQ ID NO:34 | Sung et al., 1991 |
| ribozyme arms: | ccugga ccaccg | | |
| G90D target: | gugacuu caccag | SEQ ID NO:35 | Sieving et al., 1992 |
| ribozyme arms: | cacuga gugguc | | |
| G106W target: | cgucuuc uggccc | SEQ ID NO:36 | Sung et al., 1991 |
| ribozyme arms: | gcagaa accggg | | |
| C110Y target: | caggaua caauuu | SEQ ID NO:37 | Dryja et al., 1992 |
| ribozyme arms: | guccua guuaaa | | |
| G114D target: | aggacuu cuuugc | SEQ ID NO:38 | Vaithinathan et al., 1994 |
| ribozyme arms: | uccuga gaaacg | | |
| R135G target: | aggggua cguggu | SEQ ID NO:39 | Bunge et al., 1993 |
| ribozyme arms: | ucccca gcacca | | |
| R135L target: | aguggua cguggu | SEQ ID NO:40 | Andreasson et al., 1992 |
| ribozyme arms: | ucacca gcacca | | |
| R135L target: | aguugua cguggu | SEQ ID NO:41 | Jacobson et al., 1991 |
| ribozyme arms: | ucaaca gcacca | | |
| R135P target: | agccgua cguggu | SEQ ID NO:42 | Rodriguez et al., 1993 |
| ribozyme arms: | ucggca gcacca | | |
| C140S target: | ugguguc uaagcc | SEQ ID NO:43 | Macke et al., 1993 |
| ribozyme arms: | accaca auucgg | | |
| P171L target: | accccua cucgcc | SEQ ID NO:44 | Dryja et al., 1991 |
| ribozyme arms: | ugggga gagcgg | | |
| P171L target: | ccuacuc gccggc | SEQ ID NO:45 | Dryja et al., 1991 |
| ribozyme arms: | ggauga cggccg | | |
| P171S target: | cacccuc acucgc | SEQ ID NO:46 | Stone et al., 1993 |
| ribozyme arms: | guggga ugagcg | | |
| Y178C target: | gugcauc cccgag | SEQ ID NO:47 | Farrar et al., 1991 |
| ribozyme arms: | cacgua gggcuc | | |
| P180A target: | guacauc gccgag | SEQ ID NO:48 | Daiger et al., 1995 |
| ribozyme arms: | caugua cggcuc | | |
| C187Y target: | gcucgua uggaau | SEQ ID NO:49 | Nathans et al., 1993 |
| ribozyme arms: | cgagca accuua | | |
| G188R target: | ucgugua gaaucg | SEQ ID NO:50 | Dryja et al., 1991 |
| ribozyme arms: | agcaca cuuagc | | |
| D190G target | uggaauc ggcuac | SEQ ID NO:51 | Dryja et al., 1991 |
| ribozyme arms: | accuua ccgaug | | |
| D190Y target: | gaaucua cuacua | SEQ ID NO:52 | Fishman et al., 1992 |
| ribozyme arms: | cuuaga gaugau | | |
| M207R target: | cagguuc gugguc | SEQ ID NO:53 | Farrar et al., 1992 |
| ribozyme arms: | guccaa caccag | | |
| H211R target: | cgugguc cgcuuc | SEQ ID NO:54 | Macke et al., 1993 |
| ribozyme arms: | gcacca gcgaag | | |
| H211P target: | cgugguc cccuuc | SEQ ID NO:55 | Macke et al., 1993 |
| ribozyme arms: | gcacca gggaag | | |
| C264X target: | ccugaauc ugggug | SEQ ID NO:56 | Vaithinathan et al., 1993 |
| ribozyme arms: | ggacuua acccac | | |
| P267L target: | ggugcuc uacgcc | SEQ ID NO:57 | Fishman et al., 1992 |
| ribozyme arms: | ccacga augcgg | | |
| F220C target: | uaucauc uguuuc | SEQ ID NO:58 | Bunge et al., 1993 |
| ribozyme arms: | auagua acaaag | | |
| F220C target: | cuguuuc ugcuau | SEQ ID NO:59 | Bunge et al., 1993 |
| ribozyme arms: | gacaaa acgaua | | |
| C222R target: | ucuuuuc cgcuau | SEQ ID NO:60 | Bunge et al., 1993 |
| ribozyme arms: | agacaa gcgaua | | |
| A292E target: | agaguuc uuugcc | SEQ ID NO:61 | Dryja et al., 1993 |
| ribozyme arms: | ucucaa aaacgg | | |
| Q344stop target: | cgagcua gguggc | SEQ ID NO:62 | Sung et al., 1991 |
| ribozyme arms: | gcucga ccaccu | | |
| P347S target: | uggccuc ggccua | SEQ ID NO:63 | Dryja et al., 1990 |
| ribozyme arms: | accgga ccggau | | |
| RP1 MRNA-SPECIFIC: | | | |
| R677stop target: | aaaaaauc uugaca | SEQ ID NO:64 | Pierce et al., 1999 |
| ribozyme arms: | uuuuuua aacugu | | |
| RDS/PERIPHERIN MRNA-SPECIFIC: | | | |
| C118 target: | ggcucuc ugcuuuc | SEQ ID NO:65 | Farrar et al., 1991 |
| ribozyme arms: | ccgaga acgaaag | | |
| R172Q target: | gguuuuc aggacu | SEQ ID NO:66 | Wells et al., 1993 |
| ribozyme arms: | ccaaaa uccuga | | |
| R172W target: | gguuuuu gggacu | SEQ ID NO:67 | Wells et al., 1993 |
| ribozyme arms: | ccaaaa cccuga | | |
| P210R target: | guccguu ucagcu | SEQ ID NO:68 | Jackson et al., 1993 |
| ribozyme arms: | caggca agucga | | |
| C214S target: | gcugcuc caaucc | SEQ ID NO:69 | Keen and Inglehearn, 1996 |
| ribozyme arms: | cgacga guuagg | | |
| P216L target: | aaucuua gcucgc | SEQ ID NO:70 | Kajiwara et al., 1991 |
| ribozyme arms: | uuagaa cgagca | | |
| P219 target: | cuagcuc gcggcc | SEQ ID NO:71 | Kajiwara et al., 1991 |
| ribozyme arms: | gaucga cgccgg | | |

TABLE 6
ADDITIONAL ILLUSTRATIVE HAIRPIN RIBOZYME TARGETS OF THE PRESENT INVENTION

| | |
|---|---|
| | SEQUENCE |
| RIBOZYME | Cleavage site |

| | | | | |
|---|---|---|---|---|
| RDS/PERIPHERIN MRNA-SPECIFIC: | HelixII ↓ | Helix I | SEQ ID NO: | REFERENCE |

| | | | | |
|---|---|---|---|---|
| C118..target: | ucuc u gcu | uucugc | SEQ ID NO:72 | Farrar et al., 1991 |
| Ribozyme arms: | agag aaga | aagacg | | |
| R172W target: | caac g guu | uuuggg | SEQ ID NO:73 | Wells et al., 1993 |
| Ribozyme arms: | guug aaga | aaaccc | | |
| P210R target: | cguc c guu | ucagcu | SEQ ID NO:74 | Jackson et al., 1993 |
| Ribozyme arms: | gcag aaga | agucga | | |
| C214S target: | cagc u gcu | ccaauc | SEQ ID NO:75 | Keen and Inglehearn 1996 |
| Ribozyme arms: | gucg aaga | gguuag | | |
| P216L target: | ucuu a gcu | cgccac | SEQ ID NO:76 | Kajiwara et al., 1991 |
| Ribozyme arms: | agag aaga | gcggug | | |
| P219 target: | uccu a gcu | cgcggc | SEQ ID NO:77 | Kajiwara et al., 1991 |
| Ribozyme arms: | aggg aaga | gcgccg | | |

In copending application Ser. No. 09/063,667, the inventors demonstrated that AAV-vectored ribozymes could be used as a therapy for a variety of retinal diseases, including, for example, diseases caused by the presence of mutant forms of rod opsin polypeptide-spefici mRNA. Through the use of selected ribozymes, it was demonstrated that mRNAs encoding these mutated rod opsin polypeptides could be selectively cleaved, and thus, inactivated by such AAV-vectored ribozyme compositions. In similar fashion, a series of ribozymes have been constructed and tested that are relevant to the treatment of diabetic retinopathy, a leading cause of blindness.

The chief characteristic of diabetic retinopathy is retinal neovascularization-the pathologic spread of blood vessels in the eye. Unique ribozymes have been developed which target the mRNAs that encode various proteins involved in this process. These include ribozymes directed at the wild-type mRNA for the adenosine A2b receptor, for IGF-I (insulin-like growth factor-1) receptor, for inducible nitric oxide synthase (iNOS), and for several integrins implicated in retinal neovascularization (e.g., alpha1, alpha3, alphaV). The sequences of these ribozymes and their kinetic characterization are shown in Table 7. Also shown in Table 7 are the analyses of P347S ribozymes, that are specific for another mutant form of the rod opsin polypeptide. The nucleotide sequence of each of these exemplary ribozymes is presented in Table 8.

Insulin-like growth factor-I accounts for much of the growth-stimulating properties of serum and activates cells to proceed through the cell cycle. IGF-I receptors are present in the microvascular cells of the retina, and IGF-I can induce angiogenesis (proliferation of blood vessels) in the retina in response to VEGF exposure. The migration of endothelial cells is dependent on alpha1, alpha3 and alphaV integrins, which promote cell-cell contact. Adenosine also promotes angiogenesis. Adenosine is a mediator of changes in blood flow in response to oxygen deprivation (ischemia), which may serve as the ultimate stimulus for retinal neovascularization. There are a variety of adenosine receptors in the retina that control both vasodialtion and angiogenesis. The inventors hypothesize that the A2b receptor is involved in controlling the proliferation of new blood vessels. Finally, an increase in NO (nitric oxide) appears to stimulate a disruption of the blood-retinal barrier, and this increase correlates with an increase of inducible nitric oxide synthase (iNOS or NOS2). Reducing expression of this form of nitric oxide synthase appears to prevent retinal neovascularization by maintaining the normal blood-retinal barrier.

TABLE 7

KINETIC ANALYSES OF EXEMPLARY RIBOZYME CONSTRUCTS OF THE PRESENT INVENTION

| | | | | | |
|---|---|---|---|---|---|
| Experiment | | | | | |
| No. | Ribozyme | Vₘₐₓ (nM/min) | Kₘ (nM) | k_{cat} (min − 1) | Target Sequence |

| | | | | | | | |
|---|---|---|---|---|---|---|---|
| 3382 | P347S pig | 7.8 | 1645.9 | 1.3 | AGGCGUCAGCCUA | (SEQ ID NO:78) | |
| 3386 | P347S pig | 6.7 | 1680.0 | 1.1 | | | |
| 3879 A | P347S pig | 15.8 | 2836.0 | 1.1 | | | |
| 3879 B | P347S pig | 17.7 | | 1.2 | | | |
| 3901 A | P347S pig | 27.3 | | 1.8 | | | |
| 3901 B | P347S pig | 31.9 | | 2.2 | | | |
| | | 7.3 | 2054.0 | 1.5 | | | mean |
| | | 0.5 | 553.2 | 0.4 | | | std dev |
| Anna1 | P347S human | 0.00200 | 20325.0 | 0.000163 | UGGCCUCGGCCUA | (SEQ ID NO:79) | |
| Anna2 | P347S human | 0.00070 | 63424.0 | 0.000056 | | | |
| | | 1.35E−03 | 4.19E+04 | 1.10E−04 | | | mean |
| | | 5.31E−04 | 1.76E+04 | 4.37E−05 | | | std dev |
| 3837 A | A2B Rz 1 | 666.7 | 5070.3 | 44.4 | CAUGUCUCUUUG | (SEQ ID NO:80) | |
| 3837 B | A2B Rz 1 | 416.7 | 3628.6 | 27.8 | | | |
| | | 541.7 | 4349.5 | 36.1 | | | mean |
| | | 125.0 | 720.9 | 8.3 | | | std dev |
| 3856 A | iNOS | 1.8 | 491.4 | 0.12 | GGCCUGUCCUUGGA | (SEQ ID NO:81) | |
| 3856 B | iNOS | 1.9 | 392.8 | 0.13 | | | |
| | | 1.9 | 442.1 | 0.13 | | | mean |
| | | 0.1 | 49.3 | 0.005 | | | std dev |
| 3872 A | alpha 1 Rz 1 | 61.7 | 15550 | 4.1 | AGAUGUCUAUAAG | (SEQ ID NO:82) | |
| 3872 B | alpha 1 Rz 1 | 52.4 | 35688 | 3.5 | | | |
| | | 57.0 | 25619 | 3.8 | | | mean |
| | | 4.7 | 10069 | 0.3 | | | std dev |
| 3873 A | alpha 1 Rz 2 | 31.0 | 30606.0 | 2.1 | GAGAGUCUCAUGA | (SEQ ID NO:83) | |
| 3873 B | alpha 1 Rz 2 | 53.5 | 53775.0 | 3.6 | | | |
| | | 42.2 | 42190.5 | 2.9 | | | mean |
| | | 11.3 | 11584.5 | 0.8 | | | std dev |
| 3885 A | alpha V Rz 1 | 30.5 | 4982.0 | 2.0 | GCGCGUCUUCCCG | (SEQ ID NO:84) | |
| 3885 B | alpha V Rz 1 | 36.2 | 5410.0 | 2.4 | | | |
| | | 33.4 | 5196.0 | 2.2 | | | mean |
| | | 2.9 | 214.0 | 0.2 | | | std dev |
| 3886 A | alpha V Rz 2 | 238.1 | 8537.0 | 15.9 | | | |
| 3886 B | alpha V Rz 2 | 333.3 | 14043.0 | 22.2 | ACUGGUCUUCUAC | (SEQ ID NO:85) | |
| | | 285.7 | 11290.0 | 19.1 | | | mean |
| | | 47.6 | 2753.0 | 3.1 | | | std dev |
| 3840 A | alpha 3 Rz 1 | 5.4 | 3620.0 | 0.4 | CUAUGCCUUCAUG | (SEQ ID NO:86) | |
| 3840 B | alpha 3 Rz 1 | 6.9 | 4693.0 | 0.5 | | | |
| | | 6.2 | 4156.5 | 0.4 | | | mean |
| | | 1.1 | 758.7 | 0.1 | | | std dev |
| 3841 A | alpha 3 Rz 2 | 2.8 | 1655.0 | 0.2 | CGCUGUCUUCCAC | (SEQ ID NO:87) | |
| 3841 B | alpha 3 Rz 2 | 2.2 | 1240.0 | 0.2 | | | |
| | | 2.5 | 1447.5 | 0.2 | | | mean |
| | | 0.424 | 293.449 | 0.028 | | | std dev |
| | IGF1 Rz 1 | | | | CUUCGUCUUUGCA | (SEQ ID NO:88) | |
| | IGF1 Rz 2 | 27.6 | 9757.0 | 18.4 | GUACGUCUUCCAU | (SEQ ID NO:89) | |

TABLE 8

SEQUENCE OF EXEMPLARY RIBOZYME CONSTRUCTS
OF THE PRESENT INVENTION

| | |
|---|---|
| Ribozyme | Sequence |

| | | |
|---|---|---|
| A2B Rz 1 | CAAAGACUGAUGAGCCGUUCGCGGCGAAACAUGU | (SEQ ID NO:90) |
| A2B Rz 2 | GGCAUACUGAUGAGCCGUUCGCGGCGAAACAAUG | (SEQ ID NO:91) |
| ALPHA 3 RZ 1 | CAUGAACUGAUGAGCCGUUCGCGGCGAAACAUAG | (SEQ ID NO:92) |
| ALPHA 3 RZ 2 | GUGGAACUGAUGAGCCGUUCGCGGCGAAACAGCG | (SEQ ID NO:93) |
| ALPHA 5 Rz 1 | GAGGUACUGACGAGCCGUUCGCGGCGAAACAGCA | (SEQ ID NO:94) |
| ALPHA 5 Rz 2 | GUGGCACUGAUGAGCCGUUCGCGGCGAAACAGGA | (SEQ ID NO:95) |
| ALPHA 1 Rz 1 | CUUAUACUGAUGAGCCGUUCGCGGCGAAACAUCU | (SEQ ID NO:96) |
| ALPHA 1 Rz 2 | UCAUGACUGAUGAGCCGUUCGCGGCGAAACUCUC | (SEQ ID NO:97) |
| ALPHA V Rz 1 | CGGGAACUGAUGAGCCGUUCGCGGCGAAACGCGC | (SEQ ID NO:98) |
| ALPHA V Rz 2 | GUAGAACUGAUGAGCCGUUCGCGGCGAAACCAGU | (SEQ ID NO:99) |
| IGF1 Rz 1 | UGCAAACUGAUGAGCCGUUCGCGGCGAAACGAAG | (SEQ ID NO:100) |
| IGF1 Rz 2 | GGAACUGAUGAGCCGUUCGCGGCGAAACGUAC | (SEQ ID NO:101) |
| P347S pig | UAGGCUCUGAUGAGCCGCUUCGGCGGCAAACGCCU | (SEQ ID NO:102) |
| P347S human | UAGGCCCUGAUGAGCCGCUUCGGCGGCAAAGGCCA | (SEQ ID NO:103) |
| iNOS | GCCCCAAGCUGAUGAGCCGCUUCGGCGGCGAAACAGG | (SEQ ID NO:104) |

### 5.6 Example 6

### The ROP Mouse Model for Diabetic Retinopathy

Diabetic retinopathy is characterized by the formation of new blood vessels on the surface of the retina. This neovascularization results in damage to the retina and eventual blindness. A model for diabetic retinopathy called the ROP mouse (ROP stands for retinopathy of pre-maturity) has been developed in which, under the proper conditions, neovascularization on the surface of the retina can be stimulated. These conditions are:

On postnatal day 7 the mouse pups are placed into a high oxygen (75%) environment. Then, on postnatal day 12 the mouse pups are returned to a normal oxygen environment. This lowering of the oxygen simulates a state of hypoxia within the retina of mouse pups. It is this hypoxia that stimulates the onset of neovascularization on the surface of the retina. On postnatal day 17 the mouse pups are sacrificed and their whole eyes are taken and sectioned for analysis. Using these sections, the cross sections of the new blood vessels that have formed on the surface of the retina can be readily observed. With proper staining, the nuclei of the endothelial cells that make-up these new blood vessels can also be readily counted. The extent of neovascularization is determined by the number of nuclei that can be counted on the surface of the retina per section of eye.

This ROP mouse represents an accurate and facile model system, that can be employed to test the effect of particular AAV-vectored ribozyme constructs on the formation of neovascularization on the surface of the mammalian retina.

### 5.7 Example 7

### The Insulin-Like Growth Factor 1 Receptor Ribozyme

A hammerhead ribozyme has been designed and tested in vitro and in vivo that targets and cleaves the mRNA for the insulin-like growth factor receptor (IGF-R). This ribozyme has been designated IGF1R Rz1 (SEQ ID NO:105). This ribozyme was designed and tested, in vitro, prior to cloning the gene encoding this ribozyme into the pTRUP21 adeno associated virus (AAV) vector. Once cloned into the AAV vector, the AAV-IGF1R Rz1 construct was injected into the eyes of the ROP mouse model.

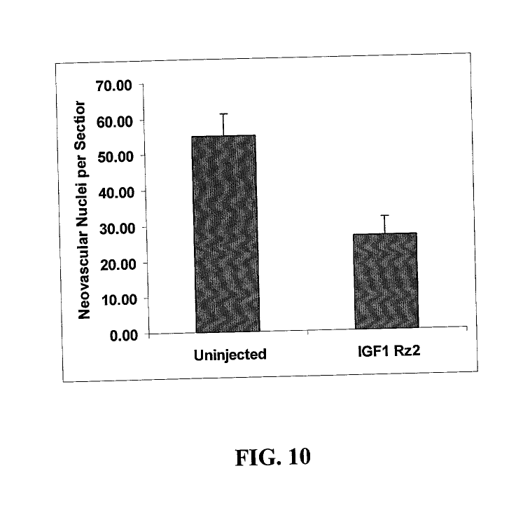

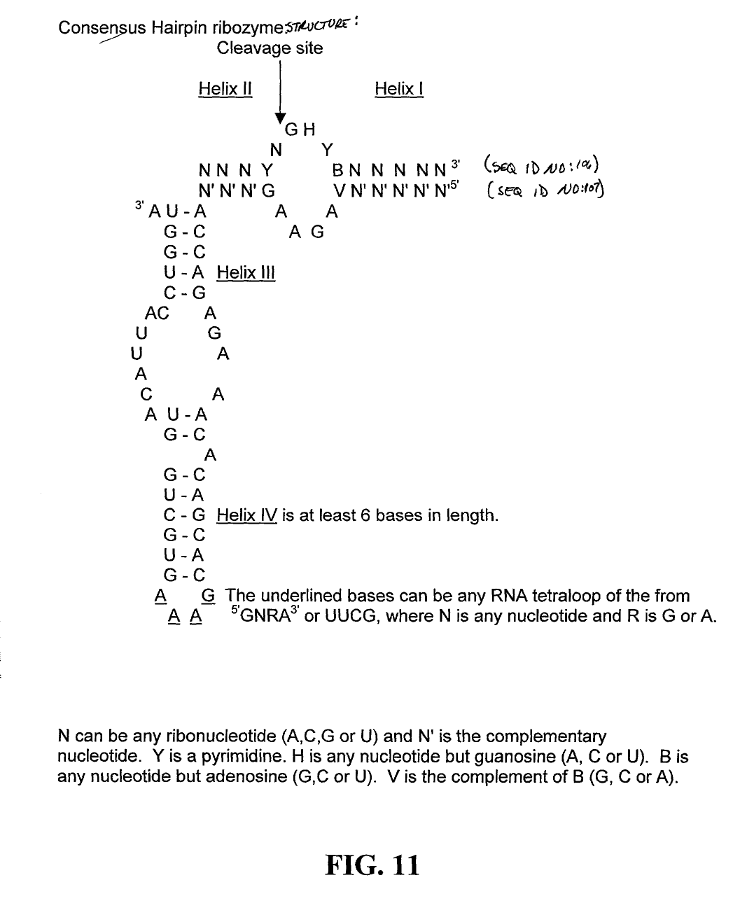

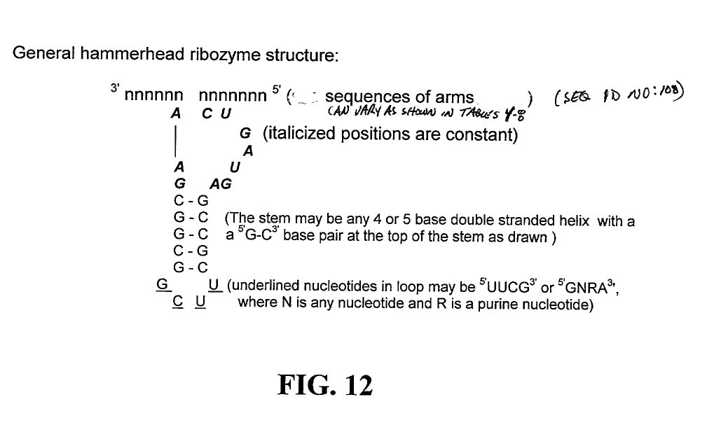

In an exemplary study, this AAV-IGF1R Rz1 construct was injected into the vitreous of the right eye the mouse pups on postnatal day 1. The left eye received no injection and served as a control. A total of 10 mice were used in this study. On postnatal day 17 the mice were sacrificed and the eyes were sectioned. The number of nuclei in endothelial cells found above the surface of the retina were counted in three sections for each eye (injected and uninjected). On average the uninjected eyes had 55 neovascular nuclei per section while the AAV-IGF1R Rz injected eyes had an average of 27 neovascular nuclei per section (FIG. 10). Statistical analyses confirmed that this finding was a “very highly significant result” with a p=3.56×10⁻⁷. The results of this study confirmed that AAV-vectored ribozyme constructs, such as IGF1R Rz1, can inhibit the formation of neovascularization on the surface of the mouse retina.

### 6. REFERENCES

The following references, to the extent that they provide exemplary procedural or other details supplementary to those set forth herein, are specifically incorporated herein by reference in whole or in part:
U.S. Pat. No. 4,683,195.
U.S. Pat. No. 4,683,202.
U.S. Pat. No. 4,800,159.
U.S. Pat. No. 4,883,750.
U.S. Pat. No. 4,987,071.
U.S. Pat. No. 5,037,746.
U.S. Pat. No. 5,093,246.
U.S. Pat. No. 5,116,742.
U.S. Pat. No. 5,297,721.
U.S. Pat. No. 5,334,711.
U.S. Pat. No. 5,354,855.
U.S. Pat. No. 5,455,166.
U.S. Pat. No. 5,631,359.
U.S. Pat. No. 5,639,655.
U.S. Pat. No. 5,646,020.
U.S. Pat. No. 5,646,031.
U.S. Pat. No. 5,648,211.
U.S. Pat. No. 5,712,124.
U.S. Pat. No. 5,744,311.
Int. Pat. Appl. No. PCT/US87/00880.
Int. Pat. Appl. No. PCT/US88/10315.
Int. Pat. Appl. No. PCT/US89/01025.
Int. Pat. Appl. Publ. No. WO 89/06700.
Int. Pat. Appl. Publ. No. WO 90/07641.
Int. Pat. Appl. Publ. No. WO 91/03162.
Int. Pat. Appl. Publ. No. WO 92/07065.
Int. Pat. Appl. Publ. No. WO 93/15187.
Int. Pat. Appl. Publ. No. WO 93/23569.
Int. Pat. Appl. Publ. No. WO 94/02595.
Int. Pat. Appl. Publ. No. WO 94/13688.
Eur. Pat. Appl. Publ. No. EP 0329822.
Eur. Pat. Appl. Publ. No. EP 0360257.
Eur. Pat. Appl. Publ. No. EP 320308.
Eur. Pat. Appl. Publ. No. EP 92110298.4.
Great Britian Pat. Appl. No. 2202328.
Adair, Montani, Strick and Guyton, “Vascular development in chick embryos: a possible role for adenosine,” Am. J. Physiol., 256:H240-46, 1989.
Agarwal, Shiraishi, Visner and Nick, “Linoleyl hydroperoxide transcriptionally upregulates heme oxygenase-1 gene expression in human renal epithelial and aortic endothelial cells,” J. Am. Soc. Nephrol., 9:1990-97, 1998.
Aiello, Avery, Arrigg, Keyt, Jampel, Shah, Pasquale, Thieme, Iwamoto, Park, et al., “Vascular endothelial growth factor in ocular fluid of patients with diabetic retinopathy and other retinal disorders,” N. Engl. J. Med., 331:1480-87, 1994.
Aiello, Bursell, Clermont, Duh, Ishii, Takagi, Mori, Ciulla, Ways, Jirousek, Smith and King, “Vascular endothelial growth factor-induced retinal permeability is mediated by protein kinase C in vivo and suppressed by an orally effective beta-isoform-selective inhibitor,” Diabetes, 46:1473-80, 1997.
Altschuler, Tritz and Hampel, Gene, 122:85-90, 1992.
Asahara, Murohara, Sullivan, Silver, van der Zee, Li, Witzenbichler, Schatteman and Isner, “Isolation of putative progenitor endothelial cells for angiogenesis,” Science, 275:964-67, 1997.
Barcz, Sommer, Sokolnicka, Gawrychowski, Roszkowska-Purska, Janik and Skopinska-Rozewska, “The influence of theobromine on angiogenic activity and proangiogenic cytokines production of human ovarian cancer cells,” Oncol. Rep., 5:517-20, 1998.
Bartlett et al., “Long-term expression of a fluorescent reproter gene via direct injection of plasmid vector into mouse skeletal muscle: Comparison of human creatine kinase and CMV promoter expression levels in vivo,” Cell Transplant., 5(3):411-419, 1996.
Beck, Powell-Braxton, Widmer, Valverde and Hefti, “Igf1 gene disruption results in reduced brain size, CNS hypomyelination, and loss of hippocampal granule and striatal parvalbumin-containing neurons,” Neuron, 14:717-30, 1995.
Bennett et al., “Adenovirus-mediated delivery of rhodopsin-promeoted bcl-2 results in a delay in photoreceptor cell death in the rd/rd mouse,” Gene Ther., 5(9):1156-1164, 1998.
Bennett, Duan, Engelhardt and Maguire, “Real-time, noninvasive in vivo assessment of adeno-associated virus-mediated retinal transduction,” Invest. Ophthalmol. Vis. Sci., 38:2857-2863, 1997.
Bennett, Maguire, Cideciyan, Schnell, Glover, Anand, Aleman, Chirmule, Gupta, Huang, Gao, Nyberg, Tazelaar, Hughes, Wilson and Jacobson, “Stable transgene expression in rod photoreceptors after recombinant adeno-associated virus-mediated gene transfer to monkey retina,” Proc. Nat'l Acad. Sci. USA, 96:9920-25, 1999.
Bikfalvi and Han, “Angiogenic factors are hematopoietic growth factors and vice versa,” Leukemia, 8:523-29, 1994.
Binley, Iqball, Kingsman, Kingsman and Naylor, “An adenoviral vector regulated by hypoxia for the treatment of ischaemic disease and cancer,” Gene Ther., 6:1721-27, 1999.
Birikh, Heaton and Eckstein, “The structure, function and application of the hammerhead ribozyme,” Eur. J. Biochem., 245:1-16, 1997.
Boast, Binley, Iqball, Price, Spearman, Kingsman, Kingsman and Naylor, “Characterization of physiologically regulated vectors for the treatment of ischemic disease,” Hum. Gene Ther., 10:2197-208, 1999.
Brown and Jampol, “New concepts of regulation of retinal vessel tone,” Arch. Ophthalmol., 114:199-204, 1996.
Buijn et al.,Science, 281:1851-1853, 1998.
Cech, Annu. Rev. Biochem., 59:543-69, 1990.
Cech, Biochem. Int., 18:7-14, 1989.
Chakravarthy, Stitt, McNally et al., Curr. Eye Res., 14:285-94, 1995.
Chen et al., “Multitarget-ribozyme directed to cleave at up to nine highly conserved HIV-1 env RNA regions inhibits HIV-1 replication—potential effectiveness against most presently sequenced HIV-1 isolates,” Nucl. Acids Res., 20:4581-4589, 1992.
Chowrira and Burke, “Extensive phosphorothioate substitution yields highly active and nuclease-resistant hairpin ribozymes,” Nucl. Acids Res., 20:2835-2840, 1992.
Cipolla, Porter and Osol, Stroke, 28:405-11, 1997.
Clark, Sferra and Johnson, “Recombinant adeno-associated viral vectors mediate long-term transgene expression in muscle,” Hum. Gene Ther., 8:659-669, 1997.
Clemmons, “IGF binding proteins: regulation of cellular actions,” Growth Regul., 2:80-87, 1992.
Cleveland, Neuron, 23:515-520, 1999.
Collins and Olive, “Reaction conditions and kinetics of self-cleavage of a ribozyme derived from Neurospora VS RNA,” Biochem., 32(11):2795-2799, 1993.
Cook and McCormick, “Inhibition by cAMP of Ras-dependent activation of Raf,” Science, 262:1069-72, 1993.
Cosentino, Hishikawa, Katusic and Luscher, Circulation, 96:25-28, 1997.
Cusi and DeFronzo, “Treatment of NIDDM, IDDM and other insulin-resistant states with IGF-I: physiological and clinical considerations,” Diabetes Rev., 3:206-36, 1995.
D'Angelo, Lee and Weiner, “cAMP-dependent protein kinase inhibits the mitogenic action of vascular endothelial growth factor and fibriblast growth factor in capillary endothelial cells by blocking Raf activation,” J Cell Biochem., 67:353-366, 1997.
Daiger, Rossiter, Greenberg, Christoffels and Hide, “Data services and software for identifying genes and mutations causing retinal degeneration,” Invest. Ophthalmol. Vis. Sci., 39:S295, 1998.
Daiger, Sullivan and Rodriguez, Behavioral Brain Sci., 18:452-67, 1995.
Daly, Vogler, Levy, Haskins and Sands, “Neonatal gene transfer leads to widespread correction of pathology in a murine model of lysosomal storage disease,” Proc. Nat 7 Acad. Sci. USA, 96:2296-300, 1999.
Damert, Ikeda and Risau, “Activator-protein-I binding potentiates the hypoxia-induciblefactor-1-mediated hypoxia-induced transcriptional activation of vascular-endothelial growth factor expression in C6 glioma cells,” Biochem. J., 327:419-23, 1997.
Davis, Szarowski, Turner, Morrisett and Shain, “In vivo activation and in situ BDNF-stimulated nuclear translocation of mitogen-activated/extracellular signal-regulated protein kinase is inhibited by ethanol in the developing rat hippocampus,” Neurosci. Lett., 272:95-98, 1999.
DesJardin and Hauswirth, Inv. Ophth. Vis. Sci., 37:154-65, 1996.
Dills, Moss, Klein and Klein, “Association of elevated IGF-I levels with increased retinopathy in late-onset diabetes,” Diabetes, 40:1725-30, 1991.
Drenser, Timmers, Hauswirth and Lewin, “Ribozyme-targeted destruction of RNAs associated with ADRP,” Inv. Ophth. Vis. Sci., 39:681-689, 1998.
Dryja and Berso, “Retinitis pigmentosa and allied diseases. Implications of genetic heterogeneity,” Invest. Ophthalmol. Vis. Sci., 36:1197-1200, 1995.
Dunn, Hardman, Kari and Barrett, “Insulin-like growth factor 1 (IGF-1) alters drug sensitivity of HBL100 human breast cancer cells by inhibition of apoptosis induced by diverse anticancer drugs,” Cancer Res., 57:2687-93, 1997.
During et al., “Peroral gene therapy of lactose intolerance using an adeno-associated virus vector,” Nature Med., 4:1131-1135, 1998.
Dusseau and Hutchins, “Hypoxia-induced angiogenesis in chick chorioallantoic membranes: a role for adenosine,” Respir. Physiol., 17:33-44, 1988.
Dusseau, Hutchins and Malbasa, “Stimulation of angiogenesis by adenosine on the chick chorioallantoic membrane,” Circ. Res., 59:163-70, 1986.
Ebert and Bunn, “Regulation of transcription by hypoxia requires a multiprotein complex that includes hypoxia-inducible factor 1, an adjacent transcription factor, and p300/CREB binding protein,” Mol. Cell Biol., 18:4089-96, 1998.
Eisen and Brown, “DNA arrays for analysis of gene expression,” Methods Enzymol., 303:179-205, 1999.
Ellis, Guberski, Somogyi-Mann and Grant, “Increased H₂O₂, vascular endothelial growth factor and receptors in the retina of the BBZ/Wor diabetic rat.” Free Radic. Biol. Med., 28:91-101, 2000.
Ethier, Chander and Dobson, Jr., “Adenosine stimulates proliferation of human endothelial cells in culture,” Am. J. Physiol., 265:H 1131-38, 1993.
Faktorovich, Steinberg, Yasamura et al., Nature, 347:83-86, 1990.
Fedor and Uhlenbeck, “Substrate sequence effects on ‘hammerhead’ RNA catalytic efficiency,” Proc. Nat'l Acad. Sci. USA, 87:1668-1672, 1990.
Fife, Bower, Cooper, Stewart, Etheridge, Coombes, Buluwela and Miller, “Endothelial cell transfection with cationic liposomes and herpes simplex-thymidine kinase mediated killing,” Gene Ther., 5:614-20, 1998.
Finkenzeller, Sparacio, Technau, Marme and Siemeister, “Sp1 recognition sites in the proximal promoter of the human vascular endothelial growth factor gene are essential for platelet-derived growth factor-induced gene expression,” Oncogene, 15:669-76, 1997.
Fisher et al., “Recombinant adeno-associated virus for muscle directed gene therapy,” Nature Med., 3:306-312, 1997.
Flamme and Risau, “Induction of vasculogenesis and hematopoiesis in vitro,” Development, 116:435-39, 1992.
Flannery, Zolotukhin, Vaquero, LaVail, Muzyczka and Hauswirth, “Efficient photoreceptor-targeted gene expression in vivo by recombinant adeno-associated virus,” Proc. Nat'l Acad. Sci. USA, 94:6916-21, 1997.
Flotte, “Stable in vivo expression of the cystic fibrosis transmembrane conductance regulator with an adeno-associated virus vector,” Proc. Natl. Acad. Sci. USA, 90:10613-10617, 1993.
Flotte, Carter, Conrad, et al., “Clinical protocol: A phase I trial of an adeno associated virus-CFTR gene vector in adult CF patients with mild lung disease,” Human Gene Therapy, 7:1145-1159, 1996.
Forsythe, Jiang, Iyer, Agani, Leung, Koos and Semenza, “Activation of vascular endothelial growth factor gene transcription by hypoxia-inducible factor 1,” Mol. Cell Biol., 16:4604-13, 1996.
Frank, “On the pathogenesis of diabetic retinopathy. A 1990 update,” Ophthalmology, 98:586-93, 1991.
Fredholm, Abbracchio, Burnstock, Daly, Harden, Jacobson, Leff and Williams, “Nomenclature and classification of purinoceptors,” Pharmacol. Rev., 46:143-56, 1994.
Frohman, In: PCR Protocols: A Guide To Methods And Applications, Academic Press, New York, 1990.
Gade, Andrades, Nemni et al., J Vasc. Surg., 26:319-26, 1997.
Gidday and Park, “Adenosine-mediated autoregulation of retinal arteriolar tone in the piglet,” Invest. Ophthalmol. Vis. Sci., 34:2713-19, 1993.
Gidday, Maceren, Shah, Meier and Zhu, “KATP channels mediate adenosine-induced hyperemia in retina,” Invest. Ophthalmol. Vis. Sci., 37:2624-33, 1996.
Gille, Swerlick and Caughman, “Transforming growth factor-alpha-induced transcriptional activation of the vascular permeability factor (VPF/VEGF) gene requires AP-2-dependent DNA binding and transactivation,” Embo. J., 16:750-59, 1997.
Gilman, In: Current Protocols in Molecular Biology, Ausubel et al., Eds., John Wiley & Sons, New York, pp. 4.7.1-4.7.8, 1987.
Gnatenko, Arnold, Zolotukhin, Nuovo, Muzyczka and Bahou, “Characterization of recombinant adeno-associated virus-2 as a vehicle for gene delivery and expression into vascular cells,” J. Investig. Med., 45:87-98, 1997.
Goldstein, Ostwald and Roth, Vision Res., 36:2979-74, 1996.
Grant and King, “IGF-1 and blood vessels,” Diabetes Rev., 3:113-28, 1995.
Grant, Caballero and Millard, “Inhibition of IGF-I and b-FGF stimulated growth of human retinal endothelial cells by the somatostatin analogue, octreotide: a potential treatment for ocular neovascularization,” Regul. Pept., 48:267-78, 1993b.
Grant, Jerdan and Merimee, “Insulin-like growth factor-I modulates endothelial cell chemotaxis,” J. Clin. Endocrinol. Metab., 65:370-71, 1987.
Grant, Mames, Fitzgerald, Ellis, Caballero, Chegini and Guy, “Insulin-like growth factor I as an angiogenic agent: in vivo and in vitro studies,” Ann. NY Acad. Sci., 692:230-42, 1993a.
Grant, Russell, Fitzgerald and Merimee, “Insulin-like growth factors in vitreous: studies in control and diabetic subjects with neovascularization,” Diabetes, 35:416-20, 1986.
Grant, Tarnuzzer, Caballero, Ozeck, Davis, Spoerri, Feoktistov, Biaggioni, Shryock and Belardinelli, “Adenosine receptor activation induces vascular endothelial growth factor in human retinal endothelial cells,” Circ. Res., 85:699-706, 1999.
Grant, Tarnuzzer, Caballero, Spoerri, Ozeck, Shryock and Belardinelli, “Adenosine mediates growth factor expression through A_{2B} adenosine receptor (AdoR) in human retinal endothelial cells (HREC),” Diabetes, 47(suppl):A39, 1998.
Grimm, Kern, Rittner and Kleinschmidt, “Novel tools for production and purification of recombinant AAV vectors,” Hum. Gene Ther., 9:2745-2760, 1998.
Guenette, Mooibroek, Wong and Tenniswood, “Cathepsin B, a cysteine protease implicated in metastatic progression, is also expressed during regression of the rat prostate and mammary glands,” Eur. J. Biochem., 226:311-21, 1994.
Guerrier-Takada, Gardiner, Marsh, pace, Altman, “The RNA moiety of ribonuclease P is the catalytic subunit of the enzyme,” Cell, 35:849, 1983.
Guy, Qi and Hauswirth, “Adeno-associated viral-mediated catalase expression suppresses optic neuritis in experimental allergic encephalomyelitis,” Proc. Nat'l Acad. Sci. USA, 95:13847-13852, 1998.
Guy, Qi, Muzyczka and Hauswirth, “Reporter expression persists 1 year after adeno-associated virus-mediated gene transfer to the optic nerve,” Arch. Ophthalmol., 117:929-37, 1999.
Hampel and Tritz, “RNA catalytic properties of the minimum (−)s TRSV sequence,” Biochem., 28:4929, 1989.
Hampel, Tritz, Hicks, Cruz, “‘Hairpin’ catalytic RNA model: evidence for helices and sequence requirement for substrate RNA,” Nucl. Acids Res., 18:299, 1990.
Hangai, Yoshimura, Hirioi, Mandai and Honda, Exp. Eye Res., 63:501-09, 1996.
Hashimoto, Kage, Ogita, Nakaoka, Matsuoka and Kira, “Adenosine as an endogenous mediator of hypoxia for induction of vascular endothelial growth factor mRNA in U-937 cells,” Biochem. Biophys. Res. Commun., 204:318-24, 1994.
Hauswirth, Lewin, Zolotukhin and Muzyczka, “Production and purification of recombinant AAV vectors,” In: Vertebrate Phototransduction and the Visual Cycle. Methods in Enzymology 316, (Palczewski (ed.)), New York, Academic Press, in press, 2000.
Hemsley, Arnheim, Toney, Cortopassi and Galas, “A simple method for site-directed mutagenesis using the polymerase chain reaction,” Nucleic Acids Res., 17:6545-51, 1989.
Hermonat and Muzyczka, “Use of adenoassociated virus as a mammalian DNA cloning vector: Transduction of neomycin resistance into mammalian tissue culture cells,” Proc. Natl. Acad. Sci. USA, 81:6466-6470, 1984.
Hertel, Herschlag and Uhlenbeck, “A kinetic and thermodynamic framework for the hammerhead ribozyme reaction,” Biochemistry, 33:3374-3385, 1994.
Hoover et al., Eds., In: Remington's Pharmaceutical Sciences, 16^{th} Edition, Mack Publishing Co., Easton, Pa., 1980.
Hyer, Sharp, Brooks, Burrin and Kohner, “A two-year follow-up study of serum insulinlike growth factor-I in diabetics with retinopathy,” Metabolism, 38:586-89, 1989.
Jacobson, Cideciyan, Huang, Hanna, Freund, Affatigato, Carr, Zack, Stone and McInnes, “Retinal degenerations with truncation mutations in the cone-rod homeobox (CRX) gene,” Invest. Ophthalmol. Vis.Sci., 39:2417-2426, 1998.
Jaeger, Turner, Zuker, “Improved predictions of secondary structures for RNA,” Proc. Natl. Acad. Sci. USA, 86(20):7706-7710, 1989.
Jager, Zhao and Porter, “Endothelial cell-specific transcriptional targeting from a hybrid long terminal repeat retrovirus vector containing human prepro-endothelin-1 promoter sequences,” J. Virol., 73:9702-09, 1999.
Joyce, “RNA evolution and the origins of life,” Nature, 338:217-244, 1989.
Kaplitt, Leone, Samulski, et al., “Long-term gene expression and phenotypic correction using adeno-associated virus vectors in the mammalian brain,” Nat. Genet., 8:148-54, 1994.
Kashani-Sabet, Funato, Tone et al., Antisense Res. Dev., 2:3-15, 1992.
Kay, Manno, Ragni, et al., “Evidence for gene transfer and expression of factor IX in haemophilia B patients treated with an AAV vector,” Nat. Genet., 24:257-261, 2000.
Kearns, Afione, Fulmer, et al., “Recombinant adeno-associated virus (AAV-CFTR) vectors do not integrate in a site-specific fashion in an immortalized epithelial cell line,” Gene Ther., 3:748-755, 1996.
Kessler, Podsakoff, Chen, McQuiston, Colosi, Matelis, Kurtzman and Byrne, “Gene delivery to skeletal muscle results in sustained expression and systemic delivery of a therapeutic protein,” Proc. Nat'l Acad. Sci. USA, 93:14082-87, 1996.
Kief and Warner, “Coordinate control of syntheses of ribosomal ribonucleic acid and ribosomal proteins during nutritional shift-up in Saccharomyces cerevisiae,” Mol. Cell Biol., 1:1007-1015, 1981.
Kimura, Weisz, Kurashima, Hashimoto, Ogura, D'Acquisto, Addeo, Makuuchi and Esumi, “Hypoxia response element of the human vascular endothelial growth factor gene mediates transcriptional regulation by nitric oxide: control of hypoxia-inducible factor-I activity by nitric oxide,” Blood, 95:189-97, 2000.
King, Goodman, Buzney, Moses and Kahn, “Receptors and growth-promoting effects of insulin and insulinlike growth factors on cells from bovine retinal capillaries and aorta,” J. Clin. Invest., 75:1028-36, 1985.
Klein, Meyer, Peel, Zolotukhin, Meyers, Muzyczka and King, “Neuron-specific transduction in the rat septohippocampal or nigrostriatal pathway by recombinant adeno-associated virus vectors,” Exp. Neurol., 150:183-94, 1998.
Koeberl, Alexander, Halbert, et al., “Persistent expression of human clotting factor 1× from mouse liver after intravenous injection of adeno-associated virus vectors,” Proc. Nat'l Acad. Sci. USA, 94:1426-1431, 1997.
Kohner and Oakley, “Diabetic retinopathy,” Metabolism, 24:1085-102, 1975.
Koizumi, Kamiya and Ohtsuka, Gene, 117:179-84, 1992.
Kolaczynski and Caro, “Insulin-like growth factor-i therapy in diabetes: physiologic basis, clinical benefits, and risks,” Ann. Intern. Med., 120:47-55, 1994.
Kvietikova, Wenger, Marti and Gassmann, “The transcription factors ATF-1 and CREB-1 bind constitutively to the hypoxia-inducible factor-I (HIF-1) DNA recognition site,” Nucleic Acids Res., 23:4542-50, 1995.
LaFace and Peck, “Reciprocal allogeneic bone marrow transplantation between NOD mice and diabetes-nonsusceptible mice associated with transfer and prevention of autoimmune diabetes,” Diabetes, 38:894-901, 1989.
Lam and Tso, Res. Commun. Mol. Pathol. Pharmacol., 92:329-40, 1996.
Langford and Miell, “The insulin-like growth factor-I binding protein axis: physiology, pathophysiology and therapeutic manipulation,” Eur. J. Clin. Invest., 23:503-16, 1993.
Lem, Applebury, Falk, Flannery and Simon, J. Biol. Chem., 266:9667-72, 1991.
Lem, Flannery, Li, et al., “Retinal degeneration is rescued in transgenic rd mice by expression of the cGMP phosphodiesterase beta subunit,” Proc. Nat'l Acad. Sci. USA, 89:44224426, 1992.
Lewin, Drenser, Hauswirth, Nishikawa, Yasumura, Flannery and LaVail, “Ribozyme rescue of photoreceptor cells in a transgenic rat model of autosomal dominant retinitis pigmentosa,” Nat. Med., 4:967-971,1998.
L'Huillier, David, Bellamy, “Cytoplasmic delivery of ribozymes leads to efficient reduction in alpha-lactalbumin mRNA levels in C1271 mouse cells,” EMBO J., 11(12):4411-4418, 1992.
Limb, Chignell, Green, LeRoy and Dumonde, “Distribution of TNF alpha and its reactive vascular adhesion molecules in fibrovascular membranes of proliferative diabetic retinopathy,” Br. J. Ophthalmol., 80:168-73, 1996.
Linden and Woo, “AAVant-garde gene therapy,” Nat. Med., 5:21-22, 1999.
Little and Lee, J. Biol. Chem., 270:9526-34, 1995.
Lutty, Mathews, Merges and McLeod, “Adenosine stimulates canine retinal microvascular endothelial cell migration and tube formation,” Curr. Eye Res., 17:594-607, 1998.
Lutty, Merges and McLeod, “5′ nucleotidase and adenosine during retinal vasculogenesis and oxygen-induced retinopathy,” Invest. Ophthalmol. Vis. Sci., 41:218-29, 2000.
Lynch, Hara, Leonard, Williams, Dean and Geary, “Adeno-associated virus vectors for vascular gene delivery,” Circ. Res., 80:497-505, 1997.
McLaughlin, Collis, Hermonat and Muzyczka, J. Virol., 62:1963-73, 1988.
Merimee, Zapf and Froesch, “Insulin-like growth factors: studies in diabetics with and without retinopathy,” N. Engl. J. Med., 309:527-30, 1983.
Meyer-Schwickerath, Pfeiffer, Blum, Freyberger, Klein, Losche, Rollmann and Schatz, “Vitreous levels of the insulin-like growth factors I and II, and the insulin-like growth factor binding proteins 2 and 3, increase in neovascular eye disease. Studies in nondiabetic and diabetic subjects,” J. Clin. Invest., 92:2620-25, 1993.
Minet, Amould, Michel, Roland, Mottet, Raes, Remacle and Michiels, “ERK activation upon hypoxia: involvement in HIF-1 activation,” FEBS Lett., 468:53-58, 2000.
Mizutani, Kern and Lorenzi, “Accelerated death of retinal microvascular cells in human and experimental diabetic retinopathy,” J. Clin. Invest., 97:2883-90, 1996.
Monahan, Samulski, Tazelaar, et al., “Direct intramuscular injection with recombinant AAV vectors results in sustained expression in a dog model of hemophilia,” Gene Ther., 5:40-49, 1998.
Morris et al., Eur. J. Neurosci., 2:1016, 1990.
Morris, Learn. Motiv., 12:239-260, 1981.
Mukai, Munekata and Higashijima, “G protein antagonists. A novel hydrophobic peptide competes with receptor for G protein binding,” J. Biol. Chem., 267:16237-43, 1992.
Muzyczka and McLaughlin, “Use of adeno-associated virus as a mamalian transduction vector,” In: Current Communications in Molecular Biology: Viral Vectors, Glzman and Hughes Eds., Cold Spring Harbor Laboratory Press, Cold Spring Harbor, N.Y., pp. 39-44, 1988.
Nakai, Herzog, Hagstrom, et al., “Adeno-associated viral vector-mediated gene transfer of human blood coagulation factor IX into mouse liver,” Blood, 91:4600-4607, 1998.
Nakai, Iwaki, Kay and Couto, “Isolation of recombinant adeno-associated virus vector-cellular DNA junctions from mouse liver,”J. Virol., 73:5438-5447, 1999.
Nathans, Thomas and Hogness, “Molecular genetics of human color vision: the genes encoding blue, green, and red pigments,” Science, 232:193-202, 1986.
Nees, Herzog, Becker, Bock, Des Rosiers and Gerlach, “The coronary endothelium: a highly active metabolic barrier for adenosine,” Basic Res. Cardiol., 80:515-29, 1985.
Nettelbeck, Jr. and Muller, “A dual specificity promoter system combining cell cycle-regulated and tissue-specific transcriptional control,” Gene Ther., 6:1276-81, 1999.
Ohkawa, Yuyama, Taira, “Activities of HIV-RNA targeted ribozymes transcribed from a ‘shot-gun’ type ribozyme-trimming plasmid,” Nucl. Acids Symp. Ser., 27:15-16, 1992.
Ojwang, Hampel, Looney, Wong-Staal, Rappaport, “Inhibition of human immunodeficiency virus type 1 expression by a hairpin ribozyme,” Proc. Natl. Acad. Sci. USA, 89(22):10802-10806, 1992.
Ostwald, Goldstein, Pachnanda and Roth, Invest. Ophthalmol. Vis. Sci., 36:2396-403, 1995.
Peel, Zolotukhin, Schrimsher, Muzyczka and Reier, Efficient transduction of green fluorescent protein in spinal cord neurons using adeno-associated virus vectors containing cell type-specific promoters.” Gene Ther., 4:16-24, 1997.
Perreault, Wu, Cousinequ, Ogilvie, Cedergren, “Mixed deoxyribo- and ribo-oligonucleotides with catalytic activity,” Nature, 344(6266):565, 1990.
Li. Perrotta and Been, “Cleavage of oligoribonucleotides by a ribozyme derived from the hepatitis delta virus RNA sequence,” Biochem., 31(1):16, 1992.
Peters, Gies, Gelb and Peterfreund, “Agonist-induced desensitization of A_{2B} adenosine receptors,” Biochem. Pharmacol., 55:873-82, 1998.
Philip et al., “Efficient and sustained gene expression in primary T lymphocytes and primary and cultured tumor cells mediated by adeno-associated virus plasmid DNA complexed to cationic liposomes,” Mol. Cell Biol., 14(4):2411-2418, 1994.
Picard and Schaffner, “A Lymphocyte-specific enhancer in the mouse immunoglobulin kappa gene,” Nature, 307:83, 1984.
Pieken, Olsen, Benseler, Aurup, Eckstein, “Kinetic characterization of ribonuclease-resistant 2′-modified hammerhead ribozymes,” Science, 253(5017):314, 1991.
Polans, Baehr and Palczewski, “Turned on by Ca2+! The physiology and pathology of Ca(2+)-binding proteins in the retina,” Trends Neurosci., 19:547-554, 1996.
Ponnazhagan, Erikson, Kearns, Zhou, Nahreini, Wang and Srivastava, “Lack of site-specific integration of the recombinant adeno-associated virus 2 genomes in human cells,” Hum. Gene Ther., 8:275-84, 1997.
Portera-Cailliau, Sung, Nathans and Adler, “Apoptotic photoreceptor cell death in mouse models of retinitis pigmentosa,” PNAS, 91:974-978, 1994.
Punglia, Lu, Hsu, Kuroki, Tolentino, Keough, Levy, Levy, Goldberg, D'Amato and Adamis, “Regulation of vascular endothelial growth factor expression by insulin-like growth factor I,” Diabetes, 46:1619-26, 1997.
Rasband and Bright, Microbeam Anal. Soc. J, 4:137-49, 1995.
Rego, Santos and Oliveira, “Oxidative stress, hypoxia, and ischemia-like conditions increase the release of endogenous amino acids by distinct mechanisms in cultured retinal cells,” J. Neurochem., 66:2506-16, 1996.
Reinhold-Hurek and Shub, “Self-splicing introns in tRNA genes of widely divergent bacteria,” Nature, 357:173-176, 1992.
Richard, Berra, Gothie, Roux and Pouyssegur, “p42/p44 mitogen-activated protein kinases phosphorylate hypoxia-inducible factor 1a (HIF-1α) and enhance the transcriptional activity of HIF-1,” J. Biol. Chem., 274:32631-37, 1999.
Ridgeway, “Mammalian expression vectors,” In: Vectors: A Survey of Molecular Cloning Vectors and Their Uses, Rodriguez and Denhardt, Eds., Stoneham: Butterworth, pp. 467-492, 1988.
Robinson, Pierce, Rook, Foley, Webb and Smith, “Oligodeoxynucleotides inhibit retinal neovascularization in a murine model of proliferative retinopathy,” Proc. Nat'l Acad. Sci. USA, 93:4851-56, 1996.
Rolling, Nong, Pisvin and Collen, “Adeno-associated virus-mediated gene transfer into rat carotid arteries,” Gene Therapy, 4:757-761, 1997.
Rosen et al., Nature, 362:59-62, 1993. Wang et al., “Efficient CFTR expression from AAV vectors packaged with promoters—the second generation,” Gene Ther., 6(4):667-675, 1999.
Rosen, Danoff, DePiero and Ziyadeh, Biochem. Biophys. Res. Commun., 207:80-88, 1995.
Rossi, Elkins, Zaia, Sullivan, “Ribozymes as anti-HIV-1 therapeutic agents: principles, applications, and problems,” AIDS Res. Hum. Retrovir., 8(2):183, 1992.
Saito, Park, Skolik, Alfaro, Chaudhry, Barnstable and Liggett, “Experimental preretinal neovascularization by laser-induced venous thrombosis in rats,” Curr. Eye Res., 16:26-33, 1997.
Salceda and Caro, “Hypoxia-inducible factor 1alpha (HIF-1α) protein is rapidly degraded by the ubiquitin-proteasome system under normoxic conditions. Its stabilization by hypoxia depends on redox-induced changes,” J. Biol. Chem., 272:22642-47, 1997.
Sambrook et al., Molecular Cloning: A Laboratory Manual, Cold Spring Harbor Laboratory, Cold Spring Harbor, N.Y., 1989.
Sanes, Rubenstein and Nicolas, EMBO J., 5:3133-42, 1986.
Sarver, Cantin, Chang, Zaia, Ladne, Stephens, Rossi, “Ribozymes as a potential anti-HIV-1 therapeutic agents,” Science, 247(4947): 1222-1225, 1990.
Saville and Collins, “A site-specific self-cleavage reaction performed by a novel RNA in Neurospora mitochondria,” Cell, 61(4):685-696, 1990.
Saville and Collins, “RNA-mediated ligation of self-cleavage products of a Neurospora mitochondrial plasmid transcript,” Proc. Natl. Acad. Sci. USA, 88(19):8826-8830, 1991.
Sawicki et al., “A composite CMV-IE enhancer/beta-actin promoter is ubiquitously expressed in mouse cutaneous epithelium,” Exp. Cell Res., 10:367-369, 1998.
Scanlon, Jiao, Funato, Wang, Tone, Rossi, Kashani-Sabet, “Ribozyme-mediated cleavage of c-fos mRNA reduces gene expression of DNA synthesis enzymes and metallothionein,” Proc. Natl. Acad. Sci. USA, 88(23):10591-10595, 1991.
Scaringe, Francklyn, Usman, “Chemical synthesis of biologically active oligoribonucleotides using beta-cyanoethyl protected ribonucleoside phosphoramidites,” Nucl. Acids Res., 18(18):5433-5441, 1990.
Selden, “Transfection using DEAE-Dextran,” in Current Protocols in Molecular Biology, Ausubel, et al. (Eds.), John Wiley & Sons: New York, pp. 9.2.1-9.2.6, 1993.
Sexl, Mancusi, Baumgartner Parzer, Schutz and Freissmuth, “Stimulation of human umbilical vein endothelial cell proliferation by A2-adenosine and beta 2-adrenoceptors,” Br. J. Pharmacol., 114:1577-86, 1995.
Sexl, Mancusi, Holler, Gloria Maercker, Schutz and Freissmuth, “Stimulation of the mitogen-activated protein kinase via the A_{2A}-adenosine receptor in primary human endothelial cells,” J. Biol. Chem., 272:5792-99, 1997.
Shaw and Lewin, “Protein-induced folding of a group I intron in cytochrome b pre-mRNA,” J. Biol. Chem., 270(37):21552-62, 1995.
Shaw, Whalen, Drenser, et al., “Ribozymes in the treatment of retinal disease,” In: Vertebrate Phototransduction and the Visual Cycle. Methods in Enzymology 316 Palezewski, Ed., New York, Academic Press, in press, 2000.
Shima, Kuroki, Deutsch, Ng, Adamis and D'Amore, “The mouse gene for vascular endothelial growth factor. Genomic structure, definition of the transcriptional unit, and characterization of transcriptional and post-transcriptional regulatory sequences,” J. Biol. Chem., 271:3877-83, 1996.
Shimayama, Nishikawa and Taira, “Generality of the NUX rule: kinetic analysis of the results of systematic mutations in the trinucleotide at the cleavage site of hammerhead ribozymes,” Biochem., 34:3649-3654, 1995.
Shryock and Belardinelli, “Adenosine and adenosine receptors in the cardiovascular system: biochemistry, physiology, and pharmacology,” Am. J. Cardiol., 79:2-10, 1997.
Simmons et al., J Histochem., 12:169-181, 1989.
Sleigh and Lockett, “SV40 enhancer activation during retinoic-acid-induced differentiation of F9 embryonal carcinoma cells,” J EMBO, 4:3831, 1985.
Smith, Kopchick, Chen, Knapp, Kinose, Daley, Foley, Smith and Schaeffer, “Essential role of growth hormone in ischemia-induced retinal neovascularization,” Science, 276:1706-09, 1997.
Smith, Shen, Perruzzi, Soker, Kinose, Xu, Robinson, Driver, Bischoff, Zhang, Schaeffer and Senger, “Regulation of vascular endothelial growth factor-dependent retinal neovascularization by insulin-like growth factor-1 receptor,” Nat. Med., 5:1390-95, 1999.
Smith, Wesolowski, McLellan, Kostyk, D'Amato, Sullivan and D'Amore, “Oxygen-induced retinopathy in the mouse,” Invest. Ophthalmol. Vis. Sci., 35:101-11, 1994.
Snyder, Miao, Meuse, et al., “Correction of hemophilia B in canine and murine models using recombinant adeno-associated viral vectors,” Nat. Med., 5:64-70, 1999.
Snyder, Miao, Patijn, Spratt, Danos, Nagy, Gown, Winther, Meuse, Cohen, Thompson and Kay, “Persistent and therapeutic concentrations of human factor IX in mice after hepatic gene transfer of recombinant AAV vectors,” Nat. Genet., 16:270-76, 1997.
Song et al., “Sustained secretion of human alpha-1-antitrypsin from muscle transduced with adeno-associated virus vectors,” Proc. Natl. Acad. Sci. USA, 24:14384-14388, 1998.
Sonksen, Russell-Jones and Jones, “Growth hormone and diabetes mellitus: a review of sixty-three years of medical research and a glimpse into the future?,” Horm. Res., 40:68-79, 1993.
Stelzner, Weil and O'Brien, “Role of cyclic adenosine monophosphate in the induction of endothelial barrier properties,” J Cell Physiol., 139:157-66, 1989.
Stephenson and Gibson, Antisense Res. Dev., 1:261-68, 1991.
Studier, Rosenberg, Dunn and Dubendorff, “Use of T7 RNA polymerase to direct expression of cloned genes,” Methods Enzymol., 185:60-89, 1990.
Takagi, King, Ferrara and Aiello, “Hypoxia regulates vascular endothelial growth factor receptor KDR/F1k gene expression through adenosine A2 receptors in retinal capillary endothelial cells,” Invest. Ophthalmol. Vis. Sci., 37:1311-21, 1996a. Takagi, King, Robinson, Ferrara and Aiello, “Adenosine mediates hypoxic induction of vascular endothelial growth factor in retinal pericytes and endothelial cells,” Invest. Ophthalmol. Vis. Sci., 37:2165-76, 1996b.
Takahashi, Kalka, Masuda, Chen, Silver, Kearney, Magner, Isner and Asahara, “Ischemia- and cytokine-induced mobilization of bone marrow-derived endothelial progenitor cells for neovascularization,” Nat. Med., 5:434-38, 1999.
Taomoto, McLeod, Merges and Lutty, “Localization of adenosine A2a receptor in retinal development and oxygen-induced retinopathy,” Invest. Ophthalmol. Vis. Sci., 41:230-43, 2000.
Taylor and Rossi, Antisense Res. Dev., 1:173-86, 1991.
Timmers, Newton and Hauswirth, “Synthesis and stability of retinal photorecptor mRNAs are coordinately regulated during bovine fetal development,” Exp. Eye Res., 56:251-265, 1993.
Tuder, Karasek and Bensch, “Cyclic adenosine monophosphate levels and the function of skin microvascular endothelial cells,” J Cell Physiol., 142:272-83, 1990.
Usman and Cedergren, “Exploiting the chemical synthesis of RNA,” Trends Biochem. Sci., 17(9):334, 1992.
Usman et al., J. Am. Chem. Soc., 109:7845-7854, 1987.
van Ginkel and Hauswirth, J. Biol. Chem., 269:4986-92, 1994.
von-Weizsacker, Blum and Wands, Biochem. Biophys. Res. Commun., 189:743-48, 1992.
Wagner, Reynolds, Moran, et al, “Efficient and persistent gene transfer of AAV-CFTR in maxillary sinus,” Lancet, 351:1702-1703, 1998.
Wu, Dent, Jelinek, Wolfman, Weber and Sturgill, “Inhibition of the EGF-activated MAP kinase signaling pathway by adenosine 3′,5′-monophosphate,” Science, 262:1065-69, 1993.
Xiao, Berta, Lu, et al., “Adeno-associated virus as a vector for liver-directed gene therapy,” J. Virol., 72:10222-10226, 1998.
Xiao, Li and Samulski, “Efficient long-term gene transfer into muscle tissue of immunocompetent mice by adeno-associated virus vector,” J. Virol., 70:8098-108, 1996.
Xiao, Li and Samulski, “Production of high-titer recombinant adeno-associated virus vectors in the absence of helper adenovirus,” J. Virol., 72:2224-2232, 1998.
Xiao, Li, McCown and Samulski, “Gene transfer by adeno-associated virus vectors into the central nervous system,” Exp. Neurol., 144:113-124, 1997.
Xing and Whitton, J. Virol., 67:1840-47, 1993.
Xu and Gong, “Adaptation of inverse PCR to generate an internal deletion,” Biotechniques, 26:639-41, 1999.
Yan, Lewin and Hauswirth. “Selective degradation of nonsense beta-phosphodiesterase mRNA in the heterozygous rd mouse,” Invest. Ophthalmol. Vis. Sci., 39:2529-2536, 1998.
Yang, Scheff and Schalch, “Effects of streptozotocin-induced diabetes mellitus on growth and hepatic insulin-like growth factor I gene expression in the rat,” Metabolism, 39:295-301, 1990. Yu, Poeschla, Yamada et al., Virology, 206:381-86, 1995.
Yarfitz and Hurley, “Transduction mechanisms of vertebrate and invertebrate photoreceptors,” J. Biol. Chem., 269:14329-14332, 1994.
Yu, Ojwang, Yamada, Hampel, Rapapport, Looney, Wong-Staal, “A hairpin ribozyme inhibits expression of diverse strains of human immunodeficiency virus type 1,” Proc. Natl. Acad. Sci. USA, 90:6340-6344, 1993.
Zolotukhin, Byrne, Mason, et al., “Recombinant adeno-associated virus purification using novel methods improves infectious titer and yield,” Gene Ther., 6:973-985, 1999. Zhou, Giordano, Durbin, McAllister, “Synthesis of functional mRNA in mammalian cells by bacteriophage T3 RNA polymerase,” Mol. Cell Biol., 10(9):4529-4537, 1990.
Zolotukhin, Potter, Hauswirth, Guy and Muzyczka, “A ‘humanized’ green fluorescent protein cDNA adapted for high-level expression in mammalian cells,” J. Virol., 70:4646-4654, 1996.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A ribozyme that specifically cleaves an mRNA encoding a polypeptide that causes or contributes to the disease, disorder, or dysfunction of a cell or a tissue of a mammalian eye.

2. The ribozyme of claim 1, wherein said ribozyme specifically cleaves an mRNA encoding a polypeptide selected from the group consisting of rod opsin, RP1, RDS/Peripherin, iNOS, A_{2B}, IGF-1, alpha 1, alpha 3, and alpha V.

3. The ribozyme of claim 2, wherein said ribozyme (a) comprises the sequence of any one of SEQ ID NO:2, or SEQ ID NO:90 to SEQ ID NO:105, or (b) specifically cleaves an mRNA comprising a sequence selected from any one of SEQ ID NO:1, or SEQ ID NO:3 to SEQ ID NO:89.

4. The ribozyme of claim 3, wherein said ribozyme comprises a sequence selected from the group consisting of SEQ ID NO:2, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO: 102, SEQ ID NO:103, SEQ ID NO:104, and SEQ ID NO:105.

5. The ribozyme of claim 2, wherein said ribozyme specifically cleaves an mRNA encoding a polypeptide selected from the group consisting of a mutant rod opsin polypeptide, a mutant RP1 polypeptide, a mutant RDS/Peripherin polypeptide, a mutant iNOS polypeptide, a mutant A_{2B} polypeptide, a mutant IGF-1 polypeptide, a mutant alpha 1 polypeptide, a mutant alpha 3 polypeptide, and a mutant alpha V polypeptide.

6. The ribozyme of claim 5, wherein said ribozyme specifically cleaves an mRNA encoding a mutant rod opsin polypeptide.

7. The ribozyme of claim 6, wherein said ribozyme specifically cleaves an mRNA encoding a mutant rod opsin polypeptide that comprises a mutation selected from the group consisting of P23H, P23L, Q28H, F45L, L46R, G51A, G51G, G51R, G51V, P53R, T58R, Q64stop, 68-71, V87D, G90D, G106W, C110Y, G114D, R135G, R135L, R135P, P171L, P171S, Y178C, P180A, C187Y, G188R, D190G, D190Y, M207R, H211R, H211P, F220C, C264X, P267L, F220C, C222R, A292E, Q344stop, and P347S.

8. The ribozyme of claim 7, wherein said ribozyme specifically cleaves an mRNA that comprises a nucleotide sequence selected from the group consisting of SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, and SEQ ID NO:63.

9. The ribozyme of claim 5, wherein said ribozyme specifically cleaves an mRNA encoding a mutant RP1 polypeptide, or an A_{2B} receptor polypeptide.

10. The ribozyme of claim 9, wherein said ribozyme specifically cleaves an mRNA comprising the sequence of SEQ ID NO:64 or SEQ ID NO:1.

11. The ribozyme of claim 5, wherein said ribozyme specifically cleaves an mRNA encoding a mutant RDS/Peripherin polypeptide.

12. The ribozyme of claim 11, wherein said ribozyme specifically cleaves an mRNA encoding a mutant RDS/Peripherin polypeptide that comprises a mutation selected from the group consisting of C118, R172Q, R172W, P210R, C214S, P216L, and P219.

13. The ribozyme of claim 12, wherein said ribozyme specifically cleaves an mRNA that comprises a sequence selected from the group consisting of SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, and SEQ ID NO:77.

14. The ribozyme of claim 1, wherein said molecule is a hammerhead ribozyme.

15. The ribozyme of claim 1, wherein said molecule is a hairpin ribozyme.

16. A vector comprising a polynucleotide encoding the ribozyme of claim 1, said polynucleotide operably linked to at least a first promoter element that directs expression of said polynucleotide in a mammalian cell.

17. The vector of claim 16, wherein said vector is a viral vector.

18. The vector of claim 17, wherein said viral vector is an adeno-associated viral vector.

19. The vector of claim 16, wherein said promoter element directs expression of said polynucleotide in a retinal cell.

20. The vector of claim 16, wherein said promoter element directs expression of said polynucleotide in a photoreceptor cell.

21. The vector of claim 16, wherein said promoter element directs expression of said polynucleotide in a rod or a cone cell.

22. The vector of claim 16, wherein said promoter element directs expression of said polynucleotide in a Mueller cell, or a retinal pigement epithelium cell.

23. The vector of claim 16, wherein said promoter element comprises a mammalian rod opsin promoter element.

24. The vector of claim 16, wherein said promoter element comprises a constitutive or an inducible promoter element.

25. A virus comprising the ribozyme of claim 1, or a polynucleotide that encodes the ribozyme of claim 1.

26. The virus of claim 25, wherein said virus is an adenovirus or an adeno-associated virus

27. An adeno-associated viral vector comprising the ribozyme of claim 1, or a polynucleotide that encodes the ribozyme of claim 1.

28. The adeno-associated viral vector of claim 27, wherein said polynucleotide is operably linked to at least a first regulatory element that directs expression of said polynucleotide in a mammalian cell.

29. The adeno-associated viral vector of claim 28, wherein said regulatory element comprises a promoter that expresses said polynucleotide in a cell of a human eye.

30. A host cell that comprises:
(a) the ribozyme of claim 1;
(b) the vector of claim 16;
(c) the virus of claim 25; or
(d) the adeno-associated viral vector of claim 27.

31. The host cell of claim 30, wherein said cell is a mammalian host cell.

32. The host cell of claim 31, wherein said mammalian host cell is a human cell.

33. The host cell of claim 32, wherein said human cell is a retinal cell.

34. The host cell of claim 33, wherein said retinal cell is a photoreceptor cell.

35. The host cell of claim 34, wherein said retinal cell is a photoreceptor rod or cone cell.

36. A composition comprising:
(a) the ribozyme of claim 1;
(b) the vector of claim 16;
(c) the virus of claim 25; or
(d) the adeno-associated viral vector of claim 27.

37. The composition of claim 36, further comprising a pharmaceutical excipient.

38. The composition of claim 37, wherein said pharmaceutical excipient is suitable for ocular or subretinal administration to a mammalian eye.

39. The composition of claim 36, further comprising a lipid, a liposome, a nanoparticle, or a microsphere.

40. A kit comprising:
(a) (i) the ribozyme of claim 1;
(ii) the vector of claim 16;
(iii) the virus of claim 25; or
(iv) the adeno-associated viral vector of claim 27; and
(b) instructions for using said kit.

41. A kit comprising the composition of claim 36, and instructions for using said kit.

42. The kit of claim 41, further comprising device for delivering said composition to the eye, retina, or subretinal space of a mammal.

43. A method for decreasing the amount of mRNA encoding a selected polypeptide in a retinal cell of a mammalian eye, comprising providing to said eye an amount of the composition of claim 36, and for a time effective to specifically cleave said mRNA in said cell, and thereby decrease the amount of mRNA in said cell.

44. The method of claim 43, wherein said ribozyme specifically cleaves an mRNA encoding a polypeptide that causes a pathological condition in, or contributes to a disease, disorder, or dysfunction in a cell or a tissue of a mammalian eye.

45. The method of claim 43, wherein said composition is provided to said eye by direct administration, ocular injection, retinal injection, or subretinal injection.

46. The method of claim 44, wherein said pathological condition is selected from the group consisting of retinal degeneration, retinitis, macular degeneration, or retinopathy.

47. The method of claim 46, wherein said retinitis is retinitis pigmentosa.

48. The method of claim 46, wherein said pathological condition is autosomal dominant retinitis pigmentosa or autosomal recessive retinitis pigmentosa.

49. The method of claim 46, wherein said pathological condition is macular degeneration.

50. The method of claim 49, wherein said pathological condition is age-related macular degeneration.

51. The method of claim 46, wherein said pathological condition is retinopathy.

52. The method of claim 51, wherein said pathological condition is diabetic retinopathy.

53. A method for decreasing the amount of a selected polypeptide in a cell or tissue of a mammalian eye, comprising providing to said eye an amount of the ribozyme of claim 1 and for a time effective to specifically decrease the amount of said selected polypeptide in said cell or said tissue.

54. A method for decreasing the amount of a selected polypeptide in the eye of a mammal suspected of having a pathological condition selected from the group consisting of retinal degeneration, retinitis, macular degeneration, and retinopathy, comprising directly administering to said eye: (a) the ribozyme of claim 1, (b) the vector of claim 16, (c) the virus of claim 25, or (d) the adeno-associated viral vector of claim 27, in an amount and for a time effective to specifically cleave an mRNA encoding said selected polypeptide, and thereby decreasing the amount of said polypeptide in said eye.

55. A method for treating, decreasing the severity, or ameliorating the symptoms of a pathological condition that results from the expression of at least a first selected polypeptide in a cell or a tissue of a human eye, said method comprising directly administering to said eye: (a) the ribozyme of claim 1, (b) the vector of claim 16, (c) the virus of claim 25, or (d) the adeno-associated viral vector of claim 27, in an amount and for a time effective to treat, decrease the severity, or ameliorate the symptoms of said pathological condition.

56. The method of claim 55, wherein said symptoms are selected from the group consisting of atrophic lesions of the eye, pigmented lesions of the eye, blindness, a reduction in central vision, a reduction in peripheral vision, and a reduction in total vision.

57. A method for decreasing the progression of a degenerative pathological condition of a mammalian eye, comprising providing to said eye: (a) the ribozyme of claim 1, (b) the vector of claim 16, (c) the virus of claim 25, or (d) the adeno-associated viral vector of claim 27, in an amount and for a time effective to decrease the progression of said degenerative pathological condition.
